(19) United States
(12) Patent Application Publication
(10) Pub. No.: US 2002/0143152 A1
(43) Pub. Date: Oct. 3, 2002

(54) Polypeptides, their production and use

(76) Inventors: Shuji Hinuma, Tsukuba-shi (JP); Shoji Fukusumi, Tsukuba-shi (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/044,592

(22) Filed: Jan. 10, 2002

Related U.S. Application Data
(63) Continuation-in-part of application No. 09/403,639, filed on Oct. 25, 1999.
Application No. 09/403,639 is a 371 of international application No. PCT/JP98/01923, filed on Apr. 27, 1998.

(30) Foreign Application Priority Data
Apr. 28, 1997 (JP) 9/109974

(51) Int. Cl. C07K 14/705; C12P 21/02; C12N 5/06

(52) U.S. Cl. 530/350; 536/23.5; 435/320.1; 435/69.1; 435/325

(57) ABSTRACT

The present invention relates to the murine- derived ligand polypeptide for the G protein-coupled receptor proteins.

The ligand polypeptide or the DNA which codes for the ligand polypeptide can be used for (1) development of medicines such as pituitary function modulators, central nervous system function modulators, and pancreatic function modulators, (2) development of receptor binding assay systems using the expression of recombinant receptor proteins and screening of pharmaceutical candidate compounds, and (3) production of non-human transgenic animals or non-human knockout animals for analyzing a function of the genes.

REFERENCE TO RELATED APPLICATIONS

This application is a continuation-in-part of U.S. patent application Ser. No. 09/403,639, filed Oct. 25, 1999, which is the National Phase filing of International Application for Patent Ser. No. PCT/JP98/01923 filed Apr.27, 1998.

### FIELD OF THE INVENTION

The present invention relates to a novel ligand polypeptide for the G protein-coupled receptor protein and a DNA comprising a DNA encoding the ligand polypeptide.

### BACKGROUND OF THE INVENTION

Many hormones and neurotransmitters mediate biological functions through specific receptors present on the cell membrane. Many of these receptors engage themselves in the intracellular transduction of signals through activation of the coupled guanine nucleotide-binding protein (hereinafter sometimes referred to briefly as G protein) and have the common structure comprising 7 transmembrane domains. Therefore, these receptors are collectively referred to as G protein-coupled receptor or 7-transmembrane receptor.

One of the pathways to modulate biological functions mediated by such hormones or neurotransmitters through G protein-coupled receptors is the hypothalamo-pituitary system. Thus, the secretion of pituitary hormones from the hypophysis is controlled by hypothalamic hormones (pituitatropic releasing factor) and the functions of the target cells or organs are regulated through the pituitary hormones released into the circulation. This pathway carries out functional modulations of importance to the living body, such as homeostasis and regulation of the reproduction, development, metabolism and growth of individuals. The secretion of pituitary hormones is controlled by a positive feedback or a negative feedback mechanism involving hypothalamic hormones and the peripheral hormone secreted from the target endocrine gland. The various receptor proteins present in the hypophysis are playing a central role in the regulation of the hypothalamus-pituitary system.

Meanwhile, it is known that these hormones and factors as well as their receptors are not localized in the hypothalamus-pituitary system but are broadly distributed in the brain. Therefore, it is suspected that, in the central nervous system, this substance called hypothalamus hormone is functioning as a neurotransmitter or a neuromodulator. Moreover, the substance is distributed in peripheral tissues as well and thought to be playing important roles in the respective tissue.

The pancreas is playing a crucial role in the carbohydrate metabolism by secreting glucagon and insulin as well as digestive juice. While insulin is secreted from the pancreatic β cells, its secretion is mainly stimulated by glucose. However, it is known that β cells have a variety of receptors and the secretion of insulin is controlled by a number of factors in addition to glucose as well as peptide hormones, e.g. galanine, somatostatin, gastric inhibitory polypeptide, glucagon, amyrin, etc.; sugars, e.g. mannose etc.; amino acids, and neurotransmitters, among others.

The means only heretofore available for identifying ligands for said G protein-coupled receptor proteins is estimation from the homology in primary structure of G protein-coupled receptor proteins.

Recently, investigation for novel opioid peptides by introducing a cDNA coding for a receptor protein for which a ligand is unknown, i.e. an orphan G protein-coupled receptor protein, into CHO cells have been reported (Reinsheid, R. K. et al., Science, 270, 792-794, 1995, Menular, J.-C., et al., Nature 377, 532-535, 1995). However, in view of similarities to known G protein-coupled receptor proteins and tissue distributions, it could be easily anticipated in these cases that the ligand would be belonging to the family of opioid peptides. The history of research and development in the realm of substances acting on the living body through the opioid receptor dates back to many years ago and various antagonists and agonists had been developed. Therefore, among the compounds artificially synthesized, an agonist of the receptor was picked out and, using it as a probe, expression of the receptor in the receptor cDNA-transfected cells was verified. Then, a search was made for an activator of the intracellular signal transduction which was similar to the agonist, and the activator so found was purified, and then the structure of the ligand was determined. However, when the homology of an orphan receptor to known G protein-coupled receptor proteins is low, little information is available on the ligand so that it is difficult to explore the ligand.

Belonging to the category of such orphan (i.e. of unknown ligand) G protein-coupled receptor proteins, a human receptor protein encoded by phGR3 (alias GPR10) gene (Genomics, 29, 335, 1995) and the corresponding rat receptor protein UHR-1 (Biochem. Biophys. Res. comun., 209, 606, 1995) are known.

### BRIEF SUMMARY OF THE INVENTION

As an approach toward elucidation of the mechanisms of onset of various diseases and establishment of pertinent therapeutics, it is relevant and rewarding to determine the ligands of orphan G protein-coupled receptor proteins which show some physiological functions or others in the living body. For example, ligands binding the orphan G protein-coupled receptor proteins expressed in the pituitary, central nervous system, and pancreatic β cells, among others, are expected to be useful drugs, although their structures and functions remain to be fully known.

Meanwhile, in order to analyze the function of a ligand, it is a very effective procedure to compare the case in which the relevant ligand gene has been expressed in excess or the case in which the gene product ligand is functionally defective with the case in which the gene expression is normal. In this connection, the most reliable method for creating a functional defect in a given gene product is to destroy the very gene. For this purpose, the gene targeting technology was developed (Thomas, K. R. et al., Cell, 51, 503-512, 1987) and many knockout mice have been constructed in the world. This technology comprises destroying a given chromosomal gene of the pluripotent murine embryonic stem cell (ES cell) by way of a homologous recombination, microinjecting the resulting ES cell into the murine blastocyst to construct chimera mice, and mating them to produce a knockout mouse. At the present time only mice are available as such knockout animal models of various diseases. In order to construct a useful transgenic mouse with a defect in the gene which appears to be associated with a given disease, the mouse genomic DNA sequence of the target gene must be known but, for the orphan G protein-coupled receptor protein pHGR3 (GPR10) or UHR-1, neither the corresponding mouse ligand peptide nor the gene (cDNA or genomic DNA) coding for the ligand peptide is known.

By using cells in which the cDNA coding for the orphan G protein-coupled receptor protein pHGR3 has been expressed and selecting a specific cell stimulating (signal transduction) activity as an indicator, the inventors of the present invention performed a screening for a bovine polypeptide which this receptor protein recognizes as its ligand and determined its amino acid sequence and DNA sequence. Then, using the DNA coding for said bovine polypeptide as a primer, the inventors isolated the bovine, human, and rat cDNAs coding for said polypeptide by polymerase chain reaction (PCR), synthesized primers based on the nucleotide sequence of the rat cDNA, and succeeded in isolating the cDNA and genomic DNA coding for the mouse polypeptide.

Furthermore, by using said ligand peptide, the inventors established a screening method for a compound which modifies the binding of the ligand to said receptor protein and further by using the genomic DNA of said mouse ligand polypeptide, constructed a non-human transgenic animal (particularly a useful knockout mouse) to thereby make it possible to analyze the function of the gene.

The present invention, therefore, relates to (1) a polypeptide comprising an amino acid sequence represented by SEQ ID NO:1, or a substantial equivalent thereto, or its amide or ester, or a salt thereof, (2) a DNA comprising a DNA having a nucleotide sequence coding for the polypeptide according to the above item (1), (3) a DNA according to the above item (2), which comprises a nucleotide sequence represented by SEQ ID NO:2 or SEQ ID NO:3, (4) a recombinant vector comprising the DNA according to the above item (2), (5) a transformant which is transformed by the DNA according to the above item (2) or the recombinant vector according to the above item (4), (6) a non-human knock out animal having an inactivated DNA of the DNA according to the above item (2), (7) a non-human transgenic animal having the DNA according to the above item (2) or its mutein, or the recombinant vector according to the above item (4), (8) a non-human animal cell having an inactivated DNA of the DNA according to the above item (2), (9) a method for producing the non-human animal cell according to the above item (8), which comprises introducing an inactivated DNA of the DNA according to the above item (2), (10) a method for producing the polypeptide according to the above item (1) or its amide or ester, or the salt thereof, which comprises cultivating the transformant according to the above item (5) to produce and accumulate the polypeptide according to the above item (1), and collecting the same, (11) a pharmaceutical composition which comprises the polypeptide according to the above item (1) or its amide or ester, or the salt thereof, and (12) an antibody against the polypeptide according to the above item (1) or its amide or ester, or the salt thereof.

Further, this invention relates to an agent for treating or preventing dementia, depression (melancholia), hyperkinetic (microencephalopathy) syndrome, disturbance of consciousness, anxiety syndrome, schizophrenia, horror, growth hormone secretory disease, hyperphagia, polyphagia, hypercholesterolemia, hyperglyceridemia, hyperlipemia, hyperprolactinemia, diabetes, cancer, pancreatitis, renal disease, Turner's syndrome, neurosis, rheumatoid arthritis, spinal injury, transient brain ischemia, amyotrophic lateral sclerosis, acute myocardial infarction, spinocerebellar degeneration, bone fracture, trauma, atopic dermatitis, osteoporosis, asthma, epilepsy, infertility and/or oligogalactia.

In this specification, the term “substantial equivalent(s)” means that the activity of the protein, e.g., nature of the binding activity of the ligand to the receptor and physical characteristics of the equivalent are substantially the same. Substitutions, deletions, additions or insertions of amino acids often do not produce radical changes in the physical and chemical characteristics of a polypeptide, in which case polypeptides containing the substitutions, deletions, additions or insertions would be considered to be substantially equivalent to polypeptides lacking the substitutions, deletions, additions or insertions. Substantially equivalent substitutes for an amino acid within the sequence may be selected from other members of the class to which the amino acid belongs. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In this specification, a mutated polypeptide which is obtained by a mutation (such as substitution, deletion, addition or insertion etc.) of the non-mutated polypeptide is a polypeptide substantially the same as the non-mutated polypeptide, wherein the physiological characteristics and biochemical characteristics of the non-mutated polypeptide is not effected by the mutation.

The polypeptide of the present invention represents a precursor polypeptide of a matured ligand polypeptide (e.g. a polypeptide comprising an amino acid sequence represented by SEQ ID NO:4 or a substantial equivalent thereof) which can bind to the G protein-coupled receptors.

For example, the polypeptide of the present invention represents a polypeptide which comprises an amino acid sequence represented by SEQ ID NO:1 or its substantial equivalent thereto, or its amide or ester, or a salt thereof (hereinafter, sometimes referred to briefly as ligand polypeptide or polypeptide). However, the polypeptide consisting of an amino acid sequence represented by SEQ ID NO:4 is excluded from the polypeptide of the present invention.

### BRIEF DESCRIPTION OF THE DRAWINGS

FIG. 1 shows the nucleotide sequence of the human pituitary-derived G protein-coupled receptor protein cDNA fragment harbored in cDNA clone p19P2 isolated by PCR using human pituitary-derived cDNA (SEQ ID NO.:63) and the amino acid (SEQ ID NO.: 64) encoded by the nucleotide sequence. The primer used for sequencing was-21M13. The underscored region corresponds to the synthetic primer.

FIG. 2 shows the nucleotide sequence of the human pituitary-derived G protein-coupled receptor protein cDNA fragment harbored in cDNA clone p19P2 isolated by PCR using human pituitary-derived cDNA (SEQ ID NO.: 65) and the amino acid sequence encoded thereby (SEQ ID NO.: 66). The primer used for sequencing was M13RV-N (Takara). The underscored region corresponds to the synthetic primer.

FIG. 3 shows a partial hydrophobic plot of the protein encoded by the human pituitary-derived G protein-coupled receptor protein cDNA fragment harbored in p19P2 constructed according to the amino acid sequence shown in FIG. 1.

FIG. 4 shows a partial hydrophobic plot of the protein encoded by the human pituitary-derived G protein-coupled receptor protein cDNA fragment harbored in p19P2 constructed according to the amino acid sequence shown in FIG. 2.

FIG. 5 is a diagram comparing the partial amino acid sequence of the protein encoded by the human pituitary-derived G protein-coupled receptor protein cDNA fragment harbored in p19P2 as shown in FIGS. 1 (SEQ ID NO.: 67) and FIG. 2 (SEQ ID NO.: 68), with the known G protein-coupled receptor protein S12863 (SEQ ID NO.: 69). The shadowed region represents the region of agreement. The 1st to 9th amino acid sequence of p19P2 corresponds to the 1st to 99th amino acid sequence of FIG. 1 and the 156th to 230th amino acid sequence corresponds to the 1st to 68th amino acid sequence of FIG. 2.

FIG. 6 shows the nucleotide sequence of the MIN6-derived G protein-coupled receptor protein cDNA fragment based on the nucleotide sequences of the MIN6-derived G protein-coupled receptor protein cDNA fragments harbored in the cDNA clones pG3-2 and pG1-10 isolated by PCR using MIN6-derived cDNA and the amino acid sequence (SEQ ID NO.: 71) encoded by the nucleotide sequence (SEQ ID NO.: 70). The underscored region corresponds to the synthetic primer.

FIG. 7 is a diagram comparing the partial amino acid sequence encoded by pG3-2/pG1-10 of the MIN6-derived G protein-coupled receptor protein shown in FIG. 6 (SEQ ID NO.: 72) with the partial amino acid sequence of the protein encoded by p19P2 shown in FIGS. 1 and 2. The shadowed region corresponds to the region of agreement. The 1st to 99th amino acid sequence of the protein encoded by p19P2 corresponds to the 1st to 99th amino acid sequence of FIG. 1 and the 156th to 223rd amino acid sequence corresponds to the 1st to 68th amino acid sequence of FIG. 2. The 1st to 223rd amino acid sequence of the protein encoded by pG3-2/pG1-10 corresponds to the 1st to 223rd amino acid sequence of FIG. 6.

FIG. 8 is a partial hydrophobic plot of the MIN6-derived G protein-coupled receptor protein constructed according to the partial amino acid sequence shown in FIG. 6.

FIG. 9 shows the entire nucleotide sequence of the human pituitary-derived G protein-coupled receptor protein cDNA harbored in the cDNA clone phGR3 isolated from a human pituitary-derived cDNA library by the plaque hybridization method using the DNA fragment inserted in p19P2 as a probe and the amino acid sequence (SEQ ID NO.: 74) encoded by the nucleotide sequence (SEQ ID NO.: 73).

FIG. 10 shows the results of Northern blotting of human pituitary mRNA hybridized with radioisotope-labeled human pituitary cDNA clone phGR3.

FIG. 11 shows a hydrophobic plot of the protein encoded by the human pituitary-derived G protein-coupled receptor protein cDNA harbored in the phGR3 as constructed according to the amino acid sequence shown in FIG. 9.

FIG. 12 shows the nucleotide sequence of the MIN6-derived G protein-coupled receptor protein cDNA fragment (SEQ ID NO.: 75) harbored in the cDNA clone p5S38 isolated by PCR using MIN6-derived cDNA and the amino acid sequence (SEQ ID NO.: 76) encoded by the nucleotide sequence. The underscored region corresponds to the synthetic primer.

FIG. 13 shows a diagram comparing the partial amino acid sequence (SEQ ID NO.: 76) of MIN6-derived G protein-coupled receptor protein encoded by p5S38 shown in FIG. 12 with the partial amino acid sequence of G protein-coupled receptor protein encoded by the cDNA fragment harbored in p19P2 as shown in FIGS. 1 and 2 and the partial amino acid sequence of G protein-coupled receptor protein encoded by the nucleotide sequence generated from the nucleotide sequences of cDNA fragments contained in pG3-2 and pG1-10 shown in FIG. 6. The shadowed region represents the sequence region of agreement. The 1st to 144th amino acid sequence of the protein encoded by p5S38 corresponds to the 1st to 144th amino acid sequence of FIG. 12, the 1st to 99th amino acid sequence of the protein encoded by p19P2 corresponds to the 1st to 99th amino acid sequence of FIG. 1 and the 156th to 223rd amino acid sequence corresponds to 1st to 68th amino acid sequence of FIG. 2. The 1st to 223rd amino acid sequence of the protein encoded by pG3-2/pG1-10 corresponds to the 1st to 223rd amino acid sequence of FIG. 6.

FIG. 14 shows a partial hydrophobic plot of the protein encoded by the MIN6-derived G protein-coupled receptor protein cDNA harbored in p5S38 as constructed according to the partial amino acid sequence shown in FIG. 12.

FIG. 15 shows the results of the following analysis. Thus, RT-PCR was carried out to confirm the expression of mRNA in CHO cells transfected by PAKKO-19P2. Lanes 1-7 represent the results obtained by performing PCRs using serial dilutions of pAKKO-19P2 for comparison, i.e. the 10 μl/ml stock solution (lane 1), ½ dilution (lane 2), ¼ dilution (lane 3), 1/64 dilution (Lane 4), 1/256 dilution (lane 5), 1/1024 dilution (lane 6), and 1/4096 dilution (lane 7) of the plasmid as templates, and analyzing the reaction mixtures by 1.2% agarose gel electrophoresis. Lanes 8 through 11 are the results obtained by performing PCRs using a 1/10 dilution (lane 8), a 1/100 dilution (lane 9), and a 1/1000 dilution (lane 10) of the cDNA prepared from the CHO-19P2 cell line as templates and subjecting the respective reaction mixtures to electrophoresis. Lane 11 was obtained by performing PCR using a template obtained by carrying out cDNA synthesis without reverse transcriptase and subjecting the PCR reaction product to electrophoresis. Lanes 12 and 13 were obtained by performing PCR using cDNAs prepared from mock CHO cells with and without addition of reverse transcriptase, respectively, as templates and subjecting the respective reaction products to electrophoresis. M represents the DNA size marker. The lanes at both ends were obtained by electrophoresing 1 μl of λ/Sty I digest (Nippon Gene) and the second lane from right was obtained with 1 μl of θ/χ174/Hinc II digest (Nippon Gene). The arrowmark indicates the position of the band amplified by PCR of about 400 bp.

FIG. 16 shows the activity of the crude ligand peptide fraction extracted from rat whole brain to promote release of arachidonic acid metabolites from CHO-19P2 cells. The arachidonic acid metabolite releasing activity was expressed as % of the amount of [³H] arachidonic acid metabolites released in the presence of the crude ligand polypeptide fraction with the amount of [³H] arachidonic acid metabolites released in the presence of 0.05% BAS-HABB being taken as 100%. The activity to promote release of arachidonic acid metabolites from the CHO-19P2 cell line was detected in a 30% CH₃CN fraction.

FIG. 17 shows the activity of the crude ligand polypeptide fraction extracted from bovine hypothalamus to promote release of arachidonic acid metabolites from CHO-19P2 cells. The arachidonic acid metabolite release-promoting activity was expressed as % of the amount of [³H] arachidonic acid metabolites released in the presence of the crude ligand polypeptide fraction with the amount of [³H] arachidonic acid metabolites released in the presence of 0.05% BAS-HABB being taken as 100%. The activity to promote release of arachidonic acid metabolites from the CHO-19P2 cell line was detected in a 30% CH₃CN fraction just as in the crude ligand polypeptide fraction from rat whole brain.

FIG. 18 shows the activity of the fraction purified with the reversed-phase column C18 218TP5415 to specifically promote release of arachidonic acid metabolites from CHO-19P2 cells. The active fraction from RESOURCE S was fractionated on C18 218TP5415. Thus, chromatography was carried out at a flow rate of 1 ml/min. on a concentration gradient of 20%-30% CH₃CN/0.1% TFA/H₂O, the eluate was collected in 1 ml fractions, and each fraction was lyophilized. Then, the activity of each fraction to specifically promote release of arachidonic acid metabolites from the CHO-19P2 cell line was determined. As a result, the activity was fractionated into 3 fractions (designated, in the order of elution, as P-1, P-2, and P-3).

FIG. 19 shows the activity of the fraction purified with the diphenyl 219TP5415 reversed-phase column to specifically promote arachidonic acid metabolite release from CHO-19P2 cells. The P-3 active fraction from C18 218TP5415 was fractionated on diphenyl 219TP5415. The chromatography was carried out at a flow rate of 1 ml/min. on a concentration gradient of 22%-25% CH₃CN/0.1% TFA/H₂O, the eluate was collected in 1 ml fractions, and each fraction was lyophilized. Then, the activity to specifically promote release of arachidonic acid metabolites from CHO-19P2 cells in each fraction was determined. As a result, the activity converged in a single peak.

FIG. 20 shows the activity of the fraction purified by μRPC C2/C18 SC 2.1/10 reversed-phase column to specifically promote release of arachidonic acid metabolites from CHO-19P2 cells. The peak active fraction from diphenyl 219TP5415 was fractionated on μRPC C2/C18 SC 2.1/10. The chromatography was carried out at a flow rate of 100 μl/min. on a concentration gradient of 22%-23.5% CH₃CN/0.1% TFA/H₂O, the eluate was collected in 100 μl fractions, and each fraction was lyophilized. Then, the activity to specifically promote release of arachidonic acid metabolites from CHO-19P2 cells in each fraction was determined. As a result, the activity was found as two peaks of apparently a single substance (peptide).

FIG. 21 shows the activity of the P-2 fraction purified by μRPC C2/C18 SC 2.1/10 reversed-phase column to specifically promote release of arachidonic acid metabolites from CHO-19P2 cells. The chromatography was carried out at a flow rate of 100 μl/min. on a concentration gradient of 21.5%-23.0% CH₃CN/0.1% TFA/dH₂O, the eluate was collected in 100 μl fractions, and each fraction was lyophilized. Then, the activity to specifically promote release of arachidonic acid metabolites from CHO-19P2 cells in each fraction was determined. As a result, the activity was found as a peak of apparently a single substance.

FIG. 22 shows the nucleotide sequence of bovine hypothalamus ligand polypeptide cDNA fragment (SEQ ID NO.: 77) as derived from the nucleotide sequence of the bovine hypothalamus-derived ligand polypeptide cDNA fragment which specifically promotes release of arachidonic acid metabolites from CHO-19P2 cells as harbored in a cDNA clone isolated by PCR using bovine hypothalamus-derived cDNA and the amino acid sequence (SEQ ID NO.: 78) encoded by said nucleotide sequence. The region indicated by the arrowmark corresponds to the synthetic primer.

FIG. 23 shows the nucleotide sequence of the bovine hypothalamus-derived ligand polypeptide cDNA fragment (SEQ ID NO.: 79) generated according to the nucleotide sequence of the bovine hypothalamus-derived ligand polypeptide cDNA fragment which specifically promotes release of arachidonic acid metabolites from CHO-19P2 cells as harbored in a cDNA clone isolated by PCR using bovine hypothalamus-derived cDNA and the amino acid sequence (SEQ ID NO.: 80) encoded by said nucleotide sequence. The region indicated by the arrowmark corresponds to the synthetic primer.

FIG. 24 shows the amino acid sequences of the bovine hypothalamus-derived ligand polypeptides which specifically promote release of arachidonic acid metabolites from CHO-19P2 cells; FIG. 24a (SEQ ID NO.: 82), FIG. 24b (SEQ ID NO.: 84), and the cDNA sequence coding for the full coding region of the ligand polypeptides defined by SEQ ID NO.: 1 and SEQ ID NO.: 44; FIG. 24a (SEQ ID NO.: 81), FIG. 24b (SEQ ID NO.: 83).

FIG. 25 shows the concentration-dependent activity of synthetic ligand polypeptide (19P2-L31) to specifically promote release of arachidonic acid metabolites from CHO-19P2 cells. The synthetic peptide was dissolved in degassed d H₂O at a final concentration of 10⁻³M and diluted with 0.05% BSA-HBSS to concentrations of 10⁻¹²M-10⁻⁶M. The arachidonic acid metabolite releasing activity was expressed in the measured radioactivity of [³H] arachidonic acid metabolites released in the supernatant when the dilution was added to the cells. As a result, the activity of 19P2-31 to specifically promote release of arachidonic acid metabolites from CHO-19P2 cells was found in a concentration-dependent manner.

FIG. 26 shows the concentration-dependent activity of synthetic ligand polypeptide (19P2-L31(O)) to specifically promote release of arachidonic acid metabolites from CHO-19P2 cells. The synthetic ligand peptide was dissolved in degassed d H₂O at a final concentration of 10⁻³M and diluted with 0.05% BSA-HBSS to concentrations of 10⁻¹²M-10⁻⁶M. The arachidonic acid metabolite releasing activity was expressed in the measured radioactivity of [³H] arachidonic acid metabolites released in the supernatant when the dilution was added to the cells. As a result, the activity of 19P2-L31(O) to specifically promote release of arachidonic acid metabolites from CHO-19P2 cells was found in a dose-dependent manner.

FIG. 27 shows the activity of synthetic ligand polypeptide 19P2-L20 to specifically promote release of arachidonic acid metabolites from CHO-19P2 cells. The synthetic peptide was dissolved in degassed distilled water at a final concentration of 10⁻³M and diluted with 0.05% BSA-HBSS to concentrations of 10⁻¹²M-10⁻⁶M. The arachidonic acid metabolite releasing activity was expressed in the measured radioactivity of [³H] arachidonic acid metabolites released in the supernatant when the dilution was added to the cells. As a result, the activity of 19P2-L20 to specifically promote release of arachidonic acid metabolites from CHO-19P2 cells was found in a dose-dependent manner.

FIG. 28 shows the 1.2% agarose gel electrophoregram of the DNA fragments of the phages cloned from a bovine genomic library as digested with restriction enzymes BamHI(B) and SalI(S). As the DNA size marker (M), StyI digests of λ phage DNA were used. In lane B, two bands derived from the vector were detected in positions between the first (19,329 bp) and second (7.743 bp) marker bands, as well as 3 bands derived from the inserted fragment between the third (6,223 bp) and 5th (3,472 bp) bands. In lane S, two bands derived from the vector were similarly detected but due to the overlap of the band of the inserted fragment, the upper band is thicker than the band in lane B.

FIG. 29 shows the nucleotide sequence around the coding region as decoded from bovine genomic DNA (SEQ ID NO.: 85). The 1st to 3rd bases (ATG) correspond to the translation start codon and the 767th to 769th bases (TAA) correspond to the translation end codon.

FIG. 30 shows a comparison between the nucleotide sequence (genome) around the coding region as deduced from bovine genomic DNA (SEQ ID NO.: 85) and the nucleotide sequence (cDNA) of bovine cDNA cloned by PCR (SEQ ID NO. :87). The sequence region of agreement is indicated by shading. As to the 101st to 572nd region, there is no corresponding region in the nucleotide sequence of cDNA, indicating that it is an intron.

FIG. 31 shows the translation of the amino acid sequence (SEQ ID NO.: 88) encoded after elimination of the intron from the nucleotide sequence around the coding region as decoded from bovine genomic DNA (SEQ ID NO.: 87).

FIG. 32 shows the full-length amino acid sequence (SEQ ID NO.: 90) and the cDNA sequence coding for the full coding region of rat ligand polypeptide (SEQ ID NO.:89).

FIG. 33 shows the amino acid sequence of bovine ligand polypeptide (SEQ ID NO.: 88) and the nucleotide sequences of DNAs coding for bovine polypeptide (SEQ ID NO.: 87) and rat polypeptide (SEQ ID NO.: 89). The arrowmark indicates the region corresponding to the synthetic primer.

FIG. 34 shows the full-length amino acid sequence and the sequence of cDNA (SEQ ID NO.: 91) coding for the full coding region of human ligand polypeptide (SEQ ID NO.: 92).

FIG. 35 shows a comparison of the amino acid sequences in the translation region of bovine ligand polypeptide (SEQ ID NO.: 88), rat ligand polypeptide (SEQ ID NO.: 90), and human ligand polypeptide (SEQ ID NO.: 92).

FIG. 36 shows the nucleotide sequence of the inserted fragment of plasmid pmGB3 (SEQ ID NO.: 93). The arrowmark indicates the sequence corresponding to the primer.

FIG. 37 shows the cDNA predicted from nucleotide sequence of plasmid pmGB3 (SEQ ID NO.: 93) and the predicted translated protein (SEQ ID NO.: 94). The arrowmark indicates the sequence corresponding to the primer. The sequence between the marks is the sequence predicted to be the intron.

FIG. 38 shows the restriction enzyme map of the ligand polypeptide of the present invention.

FIG. 39 shows (i) the nucleotide sequence coding the ligand polypeptide of the present invention and its non-coding region (SEQ ID NO.: 95), and (ii) the amino acid sequence of the ligand polypeptide of the present invention, which obtained in Example 34 (SEQ ID NO.: 96).

FIG. 40 shows the construction figure for the targeting vector pmGFEN28 obtained in Example 35.

FIG. 41 shows the result of the agarose gel electrophoresis described in Example 36, and the comparative gene map between the wild type and the recombinant (knock out) type.

### DETAILED DESCRIPTION OF THE INVENTION

The polypeptide, its amide or ester, or a salt thereof (hereinafter sometimes referred to briefly as the ligand polypeptide or the polypeptide), processes for their production, and uses for the polypeptide are now described in detail.

The above ligand polypeptide of the present invention includes any polypeptides derived from any tissues, e.g. pituitary gland, pancreas, brain, kidney, liver, gonad, thyroid gland, gall bladder, bone marrow, adrenal gland, skin, muscle, lung, digestive canal, blood vessel, heart, etc.; or cells of man and other warm-blooded animals, e.g. guinea pig, rat, mouse, swine, sheep, bovine, monkey, etc. and comprising an amino acid sequence represented by SEQ ID NO:1 or a substantial equivalent thereto. For example, in addition to the protein comprising the amino acid sequence of SEQ ID NO:1, the ligand polypeptide of the present invention includes the protein comprising an amino acid sequence having a homology of about 50-99.9%, preferably 70-99.9%, more preferably 80-99.9% and especially preferably 90-99.9% to the amino acid sequence of SEQ ID NO:1 and having qualitatively substantially equivalent activity to the protein comprising the amino acid sequence of SEQ ID NO:1. The term “substantially equivalent” means the nature of the receptor-binding activity, signal transduction activity and the like is equivalent. Thus, it is allowable that even differences among grades such as the strength of receptor binding activity and the molecular weight of the polypeptide are present.

To be more specific, the ligand polypeptide of the present invention includes the polypeptide derived from mouse and comprising the amino acid sequence of SEQ ID NO:1. In addition, the ligand polypeptide of the present invention includes the polypeptides which comprises substantially equivalent polypeptides such as (i) polypeptides wherein 1 to 15, preferably 1 to 10, and more preferably 1 to 5 amino acid residues are deleted from the amino acid sequence of SEQ ID NO:1, (ii) polypeptides wherein 1 to 80, preferably 1 to 50, more preferably 1 to 10 amino acid residues are added to the amino acid sequence of SEQ ID NO:1, or polypeptides wherein 1 to 15, preferably 1 to 10, more preferably 1 to 5 amino acid residues are substituted with other amino acid residues.

Moreover, the ligand polypeptide of the present invention includes the polypeptides wherein its constructive amino acid (especially its side chain) is modified, or its amide or ester, or a salt thereof.

The polypeptide of the present invention includes those wherein Gln of the constitutive amino acid at the N-terminal side is cleaved in vivo to form pyroglutamyl group.

The peptides described in this specification, the left end is the N-terminus (amino terminus) and the right end is the C-terminus (carboxyl terminus) according to the convention of the peptide indication. While the C-terminus of the polypeptide of SEQ ID NO:1 is usually carboxyl (—COOH) or carboxylato (—COO—), it may be amide (—CONH2) or ester (—COOR) form. The ester residue R includes a C1-6 alkyl group such as methyl, ethyl, n-propyl, isopropyl, n-butyl, etc., a C₃₋₈ cycloalkyl group such as cyclopentyl, cyclohexyl, etc., a C₆₋₁₂ aryl group such as phenyl, α-naphthyl, etc., and a C₇₋₁₄ aralkyl group such as a phenyl-C₁₋₂ alkyl group, e.g. benzyl, phenethyl, benzhydryl, etc. or an α-naphthyl-C₁₋₂ alkyl, e.g. α-naphthylmethyl etc. In addition, the ester may be a pivaloyloxymethyl ester which is broadly used for oral administration. When the polypeptide of SEQ ID NO:1 has a carboxyl or carboxylato group in any position other than the C-terminus, the corresponding amide or ester are also included in the concept of the polypeptide of the present invention. The ester mentioned just above includes the esters mentioned for the C-terminus.

The salt of polypeptide of the present invention includes salts with physiologically acceptable bases, e.g. alkali metals or acids such as organic or inorganic acids, and is preferably a physiologically acceptable acid addition salt. Examples of such salts are salts thereof with inorganic acids, e.g. hydrochloric acid, phosphoric acid, hydrobromic acid or sulfuric acid, etc. and salts thereof with organic acids, e.g. acetic acid, formic acid, propionic acid, fumaric acid, maleic acid, succinic acid, tartaric acid, citric acid, malic acid, oxalic acid, benzoic acid, methanesulfonic acid or benzenesulfonic acid, etc.

The ligand polypeptide or amide or ester, or a salt thereof of the present invention may be (i) manufactured from the tissues or cells of warm-blooded animals inclusive of human by purifying techniques or (ii) manufactured by the peptide synthesis as described hereinafter. Moreover, (iii) it can be manufactured by culturing a transformant carrying a DNA coding for the polypeptide as described hereinafter.

In the production from the tissues or cells of human or other warm-blooded animals, the ligand polypeptide can be purified and isolated by a process which comprises homogenizing the tissue or cells of human or other warm-blooded animal, extracting the homogenate with an acid, for instance, and subjecting the extract to a combination of chromatographic procedures such as reversed-phase chromatography, ion-exchange chromatography, affinity chromatography, etc.

As mentioned above, the ligand polypeptide in the present invention can be produced by per se known procedures for peptide synthesis. The methods for peptide synthesis may be any of a solid-phase synthesis and a liquid-phase synthesis. Thus, the objective peptide can be produced by condensing a partial peptide or amino acid capable of constituting the protein with the residual part thereof and, when the product has a protective group, the protective group is removed whereupon a desired peptide can be manufactured. The known methods for condensation and deprotection includes the procedures described in the following literature (1)-(5). (1) M. Bodanszky and M. A. Ondetti, Peptide Synthesis, Interscience Publishers, New York, 1966 (2) Schroeder and Luebke, The Peptide, Academic Press, New York, 1965 (3) Nobuo Izumiya et al., Fundamentals and Experiments in Peptide Synthesis, Maruzen, 1975 (4) Haruaki Yajima and Shumpei Sakakibara, Biochemical Experiment Series 1, Protein Chemistry IV, 205, 1977 (5) Haruaki Yajima (ed.), Development of Drugs-Continued, 14, Peptide Synthesis, Hirokawa Shoten

After the reaction, the protein can be purified and isolated by a combination of conventional purification techniques such as solvent extraction, column chromatography, liquid chromatography, and recrystallization. Where the protein isolated as above is in a free form, it can be converted to a suitable salt by the known method. Conversely where the isolated product is a salt, it can be converted to the free peptide by the known method.

The amide of polypeptide can be obtained by using a resin for peptide synthesis which is suited for amidation. The resin includes chloromethyl resin, hydroxymethyl resin, benzhydrylamine resin, aminomethyl resin, 4-benzyloxybenzyl alcohol resin, 4-methylbenz-hydrylamine resin, PAM resin, 4-hydroxymethylmethylphenylacetamidomethyl resin, polyacrylamide resin, 4-(2′,4′-dimethoxyphenyl- hydroxymethyl)phenoxy resin, 4-(2′,4′-dimethoxyphenyl- Fmoc aminoethyl)phenoxy resin, and so on. Using such a resin, amino acids whose α-amino groups and functional groups of side-chain have been suitably protected are It condensed on the resin according to the sequence of the objective peptide by various condensation techniques which are known per se. At the end of the series of reactions, the peptide or the A protected peptide is separated from the resin and the protective groups are removed to obtain the objective polypeptide.

For the condensation of the above-mentioned protected amino acids, a variety of activating reagents for peptide synthesis can be used but a carbodiimide compound is particularly suitable. The carbodiimide includes DCC, N,N′-diisopropylcarbodiimide, and N-ethyl-N′-(3-dimethylaminoprolyl)carbodiimide. For activation with such a reagent, a racemization inhibitor additive, e.g. HOBt and the protected amino acid are directly added to the resin or the protected amino acid pre-activated as symmetric acid anhydride, HOBt ester, or HOOBt ester is added to the resin. The solvent for the activation of protected amino acids or condensation with the resin can be properly selected from among those solvents which are known to be useful for peptide condensation reactions. For example, N,N- dimethylformamide, N-methylpyrrolidone, chloroform, trifluoroethanol, dimethyl sulfoxide, DMF, pyridine, dioxane, methylene chloride, tetrahydrofuran, acetonitrile, ethyl acetate, or suitable mixtures of them can be mentioned. The reaction temperature can be selected from the range hitherto-known to be useful for peptide bond formation and is usually selected from the range of about −20° C.-50° C. The activated amino acid derivative is generally used in a proportion of 1.5-4 fold excess. If the condensation is found to be insufficient by a test utilizing the ninhydrin reaction, the condensation reaction can be repeated to achieve a sufficient condensation without removing the protective group. If repeated condensation still fails to provide a sufficient degree of condensation, the unreacted amino group can be acetylated with acetic anhydride or acetylimidazole.

The protecting group of amino group for the starting material amino acid includes Z, Boc, tertiary- amyloxycarbonyl, isobornyloxycarbonyl, 4-methoxybenzyloxycarbonyl, Cl-Z, Br-Z, adamantyloxycarbonyl, trifluoroacetyl, phthalyl, formyl, 2-nitrophenylsulfenyl, diphenylphosphinothioyl, or Fmoc. The carboxy-protecting group that can be used includes but is not limited to the above-mentioned C₁₋₆ alkyl, C₃₋₈ cycloalkyl and C₇₋₁₄ aralkyl as well as 2-adamantyl, 4-nitrobenzyl, 4-methoxybenzyl, 4-chlorobenzyl, phenacyl, benzyloxycarbonylhydrazido, tertiary-butoxycarbonylhydrazido, and tritylhydrazido.

The hydroxy group of serine and threonine can be protected by esterification or etherification. The group suited for said esterification includes carbon-derived groups such as lower alkanoyl groups, e.g. acetyl etc., aroyl groups, e.g. benzoyl etc., benzyloxycarbonyl, and ethoxycarbonyl. The group suited for said etherification includes benzyl, tetrahydropyranyl, and tertiary-butyl.

The protective group for the phenolic hydroxyl group of tyrosine includes Bzl, Cl₂-Bzl, 2-nitrobenzyl, Br-Z, and tertiary-butyl.

The protecting group for imidazole moiety of histidine includes Tos, 4-methoxy-2,3,6-trimethylbenzenesulfonyl, DNP, benzyloxymethyl, Bum, Boc, Trt, and Fmoc.

The activated carboxyl group of the starting amino acid includes the corresponding acid anhydride, azide, and active esters, e.g. esters with alcohols such as pentachlorophenol, 2,4,5-trichlorophenol, 2,4-dinitrophenol, cyanomethyl alcohol, p-nitrophenol, HONB, N-hydroxysuccinimide, N-hydroxyphthalimide, HOBt, etc. The activated amino group of the starting amino acid includes the corresponding phosphoramide.

The method for elimination of protective groups includes catalytic reduction using hydrogen gas in the presence of a catalyst such as palladium black or palladium-on-carbon, acid treatment with anhydrous hydrogen fluoride, methanesulfonic acid, trifluoromethanesulfonic acid, trifluoroacetic acid, or a mixture of such acids, base treatment with diisopropylethylamine, triethylamine, piperidine, piperazine, reduction with sodium metal in liquid ammonia. The elimination reaction by the above-mentioned acid treatment is generally carried out at a temperature of −20° C.-40° C. and can be conducted advantageously with addition of a cation acceptor such as anisole, phenol, thioanisole, m-cresol, p-cresol, dimethyl sulfide, 1,4-butanedithiol, 1,2-ethanedithiol. The 2,4-dinitrophenyl group used for protecting the imidazole group of histidine can be eliminated by treatment with thiophenol, while the formyl group used for protecting the indole group of tryptophan can be eliminated by alkali treatment with dilute sodium hydroxide solution or dilute aqueous ammonia as well as the above-mentioned acid treatment in the presence of 1,2-ethanedithiol, 1,4-butanedithiol.

The method for protecting functional groups which should not take part in the reaction of the starting material, the protective groups that can be used, the method of removing the protective groups, and the method of activating the functional groups that are to take part in the reaction can all be selected from among the known groups and methods.

Another method for obtaining the amide form of the polypeptide comprises amidating the α-carboxyl group of the C-terminal amino acid at first, then extending the peptide chain to the N-side until the desired chain length, and then selectively deprotecting the a-amino group of the C-terminal peptide and the α-carboxy group of the amino acid or peptide that is to form the remainder of the objective polypeptide and condensing the two fragments whose α-amino group and side-chain functional groups have been protected with suitable protective groups mentioned above in a mixed solvent such as that mentioned hereinbefore. The parameters of this condensation reaction can be the same as described hereinbefore. From the protected peptide obtained by condensation, all the protective groups are removed by the above-described method to thereby provide the desired crude peptide. This crude peptide can be purified by known purification procedures and the main fraction lyophilized to provide the objective amidated polypeptide.

To obtain an ester of the polypeptide, the α-carboxyl group of the C-terminal amino acid is condensed with a desired alcohol to give an amino acid ester and then, the procedure described above for production of the amide is followed.

The ligand polypeptide of the present invention, its amide or ester, or a salt thereof can be any peptide that has the same activities, e.g. pituitary function modulating activity, central nervous system function modulating activity, or pancreatic function modulating activity as the polypeptide which has an amino acid sequence of SEQ ID NO:1 or substantial equivalent thereto. Among such peptides,

(1) Ser-Arg-Ala-His-Gln-His-Ser-Met-Glu-Thr-Arg-Thr-Pro-Asp-Ile-Asn-Pro-Ala-Trp-Tyr-Thr-Gly-Arg-Gly-Ile-Arg-Pro-Val-Gly-Arg-Phe (SEQ ID NO:5) and

(2) Thr-Pro-Asp-Ile-Asn-Pro-Ala-Trp-Tyr-Thr-Gly-Arg-Gly-Ile-Arg-Pro-Val-Gly-Arg-Phe (SEQ ID NO:6) are preferable.

The ligand polypeptide or partial peptide thereof can be used as antigen for preparation of anti-ligand polypeptide antibody. The preferable polypeptide as antigen includes

(1) Ser-Arg-Ala-His-Gln-His-Ser-Met-Glu (SEQ ID NO:7),

(2) Thr-Pro-Asp Ile-Asn-Pro-Ala-Trp-Tyr (SEQ ID NO:8) and

(3) Gly-Arg-Gly-Ile-Arg-Pro-Val-Gly-Arg-Phe (SEQ ID NO:9).

The ligand peptide may be a peptide containing each of the domains which can act as an antigenic determinant or a peptide containing a plurality of the domains within the molecule.

The ligand peptide mentioned in this specification may be one ending with an amide bond (—CONH₂) or an ester bond (—COOR) at the C-terminus. The ester here includes the same one of the above polypeptide. When the ligand peptide has a carboxyl or carboxylato group in any position other than the C-terminus, the case in which such group or moiety has been amidated or esterified also falls within the scope of the ligand peptide in the present invention. The ester here may be of the same one as the above-mentioned ester at the C-terminus.

The ligand polypeptide or its partial peptide of the present invention may be in the form of a fused protein which is fused with a protein whose functions or properties are already known.

The salt of such partial peptide of the ligand polypeptide of present invention may be of the same one as the above-mentioned salt of the polypeptide.

The partial peptide of the ligand polypeptide of the invention, its amide or ester, or a salt thereof can be produced by the same synthetic processes as mentioned for the polypeptide or by cleaving the polypeptide of the present invention with a suitable peptidase.

The DNA coding for the ligand polypeptide or a partial peptide thereof of the present invention may be any DNA comprising the nucleotide sequence encoding a polypeptide having an amino acid sequence of SEQ ID NO:1 or substantial equivalent thereto. Furthermore, the DNA may be any of genomic DNA, genomic DNA library, tissue- or cell-derived cDNA, tissue- or cell-derived cDNA library, and synthetic DNA. The vector for such a library may be any of bacteriophage, plasmid, cosmid, and phagimid. Moreover, it can be directly amplified by the RT-PCR (reverse transcription polymerase chain reaction) method by using an RNA fraction may be prepared from a tissue or cells.

To be more specific, as the DNA coding for a polypeptide comprising the amino acid sequence of SEQ ID NO:1, the cDNA comprising the nucleotide sequence of SEQ ID NO:2 or the genomic DNA comprising the nucleotide sequence of SEQ ID NO:3 can be exemplified.

Among DNAs coding for the mouse-derived polypeptide comprising the amino acid sequence of SEQ ID NO:1, DNA fragments comprising partial nucleotide sequences of 6 to 90, preferably 6 to 60, more preferably 9 to 30, and especially preferably 12 to 30 can be advantageously used as DNA probes as well.

The DNA coding for the ligand polypeptide of the present invention can be produced by the following genetic engineering procedures.

The DNA fully encoding the polypeptide or partial peptide of the present invention can be cloned either by PCR amplification using synthetic DNA primers having a partial nucleotide sequence of the polypeptide or partial peptide or by hybridization using the DNA inserted in a suitable vector and labeled with a DNA fragment comprising a part or full region of a murine- derived polypeptide or a synthetic DNA. The hybridization can be carried out typically by the procedure described in Molecular Cloning (2nd ed., J. Sambrook et al., Cold Spring Harbor Lab. Press, 1989). When a commercial library is used, the instructions given in the accompanying manual can be followed.

The cloned DNA coding for the polypeptide or partial go peptide can be used directly or after digestion with a restriction enzyme or addition of a linker depending on purposes. This DNA has ATG as the translation initiation codon at the 5′ end and may have TAA, TGA, or TAG as the termination codon at the 3′ end. The translation initiation and termination codons can be added by means of suitable DNA adapters.

An expression vector for the polypeptide or partial peptide can be produced by, for example (a) cutting out a target DNA fragment from the DNA for the polypeptide or partial peptide of the present invention and (b) ligating the target DNA fragment with the downstream side of a promoter in a suitable expression vector.

The vector may include plasmids derived from Escherichia coli, e.g., pBR322, pBR325, pUC12, pUC13, etc.; plasmids derived from Bacillus subtilis, e.g., pUB110, pTP5, pC194, etc.; plasmids derived from yeasts e.g., pSH19, pSH15, etc.; bacteriophages such as λ-phage, and animal viruses such as retrovirus, vaccinia virus and baculovirus.

According to the present invention, any promoter can be used as long as it is compatible with the host cell in which it is used for expressing a gene. When the host for the transformation is E. coli, the promoters are preferably trp promoters, lac promoters, recA promoters, λ_{PL} promoters, lpp promoters, etc. When the host for the transformation is Bacillus, the promoters are preferably SPO1 promoters, SPO2 promoters, penP promoters, etc. When the host is a yeast, the promoters are preferably PHO5 promoters, PGK promoters, GAP promoters, ADH promoters, etc. When the host is an animal cell, the promoters include SV40-derived promoters, retrovirus promoters, metallothionein promoters, heat shock promoters, cytomegalovirus (CMV) promoters, SRα promoters, etc. An enhancer can be effectively utilized for expression.

As required, furthermore, a host-compatible signal sequence is added to the N-terminal side of the polypeptide or partial peptide thereof. When the host is E. coli, the utilizable signal sequences may include alkaline phosphatase signal sequence, OmpA signal sequence, etc. When the host is Bacillus, they may include α-amylase signal sequence, subtilisin signal sequence, etc. When the host is a yeast, they may include mating factor α signal sequence, invertase signal sequence, etc. When the host is an animal cell, they may include insulin signal sequence, α-interferon signal sequence, antibody molecule signal sequence, etc.

A transformant or transfectant is produced by using the vector thus constructed, which carries the polypeptide- or partial peptide-encoding DNA of the present invention. The host may be, for example, Escherichia microorganisms, Bacillus microorganisms, yeasts, insect cells, animal cells, etc. Examples of the Escherichia and Bacillus microorganisms include Escherichia coli K12.DH1 (Proc. Natl. Acad. Sci. USA, Vol. 60, 160 (1968)), JM103 (Nucleic Acids Research, Vol. 9, 309 (1981)), JA221 (Journal of Molecular Biology, Vol. 120, 517 (1978)), HB101 (Journal of molecular Biology, Vol, 41, 459 (1969)), C600 (Genetics, Vol. 39, 440 (1954)), etc. Examples of the Bacillus microorganism are, for example Bacillus subtilis MI114 (Gene, Vol. 24, 255 (1983)), 207-21 (Journal of Biochemistry, Vol. 95, 76 (1984)), etc. The yeast may be, for example, Saccharomyces cerevisiae AH22, AH22R-, NA87-11A, DKD-5D, 20B-12, etc. The insect may include a silkworm (Bombyx mori larva) (Maeda et al, Nature, Vol. 315, 592 (1985)) etc. The host animal cell may be, for example, monkey-derived cell line, COS-7, Vero, Chinese hamster ovary cell line (CHO cell), DHFR gene-deficient Chinese hamster cell line (dhfr-CHO cell), mouse L cell, mouse myeloma cell, human FL, etc.

Depending on the host cell used, transformation is done using standard techniques appropriate to such cells. Transformation of Escherichia microorganisms can be carried out in accordance with the methods as disclosed in, for example, Proc. Natl. Acad. Sci. USA, Vol. 69, 2110 (1972), Gene, Vol. 17, 107 (1982), etc. Transformation of Bacillus microorganisms can be carried out in accordance with the methods as disclosed in, for example, Molecular & General Genetics, Vol. 168, 111 (1979), etc. Transformation of the yeast can be carried out in accordance with the methods as disclosed in, for example, Proc. Natl. Acad. Sci. USA, Vol. 75, 1929 (1978), etc. The insect cells can be transformed in accordance with the methods as disclosed in, for example, Bio/Technology, 6, 47-55, 1988. The animal cells can be transformed by the methods as disclosed in, for example, Virology, Vol. 52, 456, 1973, etc. The transformants or transfectants which harbor the expression vector carrying DNA encoding a polypeptide or partial peptide thereof are produced according to the aforementioned techniques.

Cultivation of the transformant (transfectant) in which the host is Escherichia or Bacillus microorganism can be carried out suitably in a liquid culture medium. The culture medium may contain carbon sources, nitrogen sources, minerals, etc. necessary for growing the transformant. The carbon source may include glucose, dextrin, soluble starch, sucrose, etc. The nitrogen source may include organic or inorganic substances such as ammonium salts, nitrates, corn steep liquor, peptone, casein, meat extracts, bean-cakes, potato extracts, etc. Examples of the minerals may include calcium chloride, sodium dihydrogen phosphate, magnesium chloride, etc. It is further allowable to add yeast extracts, vitamins, growth-promoting factors, etc. It is desired that the culture medium is pH from about 5 to about 8.

The Escherichia microorganism culture medium is preferably an M9 medium containing, for example, glucose and casamino acids (Miller, Journal of Experiments in Molecular Genetics), 431-433, Cold Spring Harbor Laboratory, New York, 1972. Depending on necessity, the medium may be supplemented with drugs such as 3β-indolyl acrylic acid in order to improve efficiency of the promoter. In the case of an Escherichia host, the cultivation is carried out usually at about 15 to 43° C. for about 3 to 24 hours. As required, aeration and stirring may be applied. In the case of Bacillus host, the cultivation is carried out usually at about 30 to 40° C. for about 6 to 24 hours. As required, aeration and stirring may be also applied. In the case of the transformant in which the host is a yeast, the culture medium used may include, for example, a Burkholder minimum medium (Bostian, K. L. et al., Proc. Natl. Acad. Sci. USA, Vol. 77, 4505 (1980)), an SD medium containing 0.5% casamino acids (Bitter, G. A. et al., Proc. Natl. Acad. Sci. USA, Vol. 81, 5330 (1984)), etc. It is preferable that the pH of the culture medium is adjusted to be from about 5 to about 8. The cultivation is carried out usually at about 20 to 35° C. for about 24 to 72 hours. As required, aeration and stirring may be applied. In the case of the transformant in which the host is an insect, the culture medium used may include those obtained by suitably adding additives such as inactivated (or immobilized) 10% bovine serum and the like to the Grace's insect medium (Grace, T. C. C., Nature, 195, 788 (1962)). It is preferable that the pH of the culture medium is adjusted to be about 6.2 to 6.4. The cultivation is usually carried out at about 27 C. for about 3 to 5 days. As desired, aeration and stirring may be-applied. In the case of the transformant in which the host is an animal cell, the culture medium used may include MEM medium (Science, Vol. 122, 501 (1952)), DMEM medium (Virology, Vol. 8, 396 (1959)), RPMI 1640 medium (Journal of the American Medical Association, Vol. 199, 519 (1967)), 199 medium (Proceedings of the Society of the Biological Medicine, Vol. 73, 1 (1950)), etc. which are containing, for example, about 5 to 20% of fetal calf serum. It is preferable that the pH is from about 6 to about 8. The cultivation is usually carried out at about 30 to 40 C. for about 15 to 60 hours. As required, medium exchange, aeration and stirring may be applied.

Separation and purification of the polypeptide or partial peptide from the above-mentioned cultures can be carried out according to methods described herein below.

To extract the polypeptide or partial peptide from the cultured microorganisms or cells, the microorganisms or cells are collected by known methods after the cultivation, suspended in a suitable buffer solution, disrupted by ultrasonic waves, lysozyme and/or freezing and thawing, etc. and, then, a crude extract of the polypeptide or partial peptide is obtained by centrifugation or filtration. Other conventional extracting or isolating methods can be applied. The buffer solution may contain a protein-denaturing agent such as urea or guanidine hydrochloride or a surfactant such as Triton X-100 (registered trademark, hereinafter often referred to as “TM”).

In the case where the polypeptide or partial peptide is secreted into culture media, supernatant liquid is separated from the microorganisms or cells after the cultivation is finished and the resulting supernatant liquid is collected by widely known methods. The culture supernatant liquid and extract containing the polypeptide or partial peptide can be purified by a suitable combination of widely known methods for separation, isolation and purification. The widely known methods of separation, isolation and purification may include methods which utilizes solubility, such as salting out or sedimentation with solvents, methods which utilizes chiefly a difference in the molecular size or weight, such as dialysis, ultrafiltration, gel filtration and SDS-polyacrylamide gel electrophoresis, methods utilizing a difference in the electric charge, such as ion-exchange chromatography, methods utilizing specific affinity such as affinity chromatography, methods utilizing a difference in the hydrophobic property, such as reverse-phase high-performance liquid chromatography, and methods utilizing a difference in the isoelectric point such as isoelectric electrophoresis, or chromatofocusing, etc.

In cases where the polypeptide or partial peptide thus obtained is in a free form, the free protein can be converted into a salt thereof by known methods or method analogous thereto. In case where the polypeptide or partial peptide thus obtained is in a salt form vice versa, the protein salt can be converted into a free form or into any other salt thereof by known methods or method analogous thereto.

The polypeptide or partial peptide produced by the transformant can be arbitrarily modified or a polypeptide can be partly removed therefrom, by the action of a suitable protein-modifying enzyme before or after the purification. The protein-modifying enzyme may include trypsin, chymotrypsin, arginyl endopeptidase, protein kinase, glycosidase, etc. The activity of the polypeptide or partial peptide thus formed can be measured by experimenting the coupling (or binding) with receptor or by enzyme immunoassays (enzyme linked immunoassays) using specific antibodies.

The DNA coding for the ligand polypeptide of the present invention, the ligand polypeptide or a partial peptide thereof can be used for (1) synthesis of a part or the full length of the ligand for G protein-coupled receptor protein, (2) search for the physiological activities of the ligand polypeptide or partial peptide thereof of the present invention, (3) preparation of a synthetic oligonucleotide probe or a PCR primer, (4) acquisition of DNAs coding for ligands of G protein-coupled receptor proteins and precursor proteins, (5) development of receptor-binding assay systems using the expression of recombinant receptor proteins and screening of candidates for medicinally active compounds, (6) acquisition of antibodies and antisera, (7) development of diagnostic agents utilizing said antibodies or antisera, (8) development of drugs such as pituitary function modulators, central nervous system function modulators, and pancreatic function modulators, and (9) gene therapies, among other uses.

Particularly the DNA comprising the DNA coding the ligand polypeptide of this invention is useful for a production of a non-human transgenic animal or a knock out mouse and for analyzing a physiological function or a functional mechanism of the ligand polypeptide by using the non-human transgenic animal or the knock out mouse.

Further, referring to (8) above, the ligand poly-peptide, or the DNA encoding either of them of the present invention is useful as a safe pharmaceutical composition of low toxic potential because it is recognized as a ligand by the G protein-coupled receptor protein expressed in the hypophysis, central nervous system and pancreatic β cells. The ligand polypeptide, a partial peptide thereof, or the DNA encoding either of them of the present invention is associated with the modulation of pituitary function, central nervous system function, and pancreatic function and, therefore, can be used as a pharmaceutical composition for treatment or prevention of dementia such as senile dementia, cerebrovascular dementia (dementia due to cerebrovascular disorder), dementia associated with phylodegenerative retroplastic diseases (e.g. Alzheimer's disease, Parkinson's disease, Pick's disease, Huntington's disease, etc.), dementia due to infectious diseases (e.g. delayed viral infections such as Creutzfelt-Jakob disease), dementia associated with endocrine, metabolic, and toxic diseases (e.g. hypothyroidism, vitamin B12 deficiency, alcoholism, and poisoning due to various drugs, metals, or organic compounds), dementia associated with oncogenic diseases (e.g. brain tumor), dementia due to traumatic diseases (e.g. chronic subdural hematoma):, depression (melancholia), hyperkinetic (microencephalopathy) syndrome, disturbance of consciousness, anxiety syndrome, schizophrenia, horror, growth hormone secretory disease (e.g. gigantism, acromegalic gigantism etc.), hyperphagia, polyphagia, hypercholesterolemia, hyperglyceridemia, hyperlipemia, hyperprolactinemia, diabetes (e.g. diabetic complications, diabetic nephropathy, diabetic neuropathy, diabetic retinopathy etc.), cancer (e.g. mammary cancer, lymphatic leukemia, cystic cancer, ovary cancer, prostatic cancer etc.), pancreatitis, renal disease (e.g. chromic renal failure, nephritis etc.), Turner's syndrome, neurosis, rheumatoid arthritis, spinal injury, transient brain ischemia, amyotrophic lateral sclerosis, acute myocardial infarction, spinocerebellar degeneration, bone fracture, trauma, atopic dermatitis, osteoporosis, asthma, epilepsy, infertility or oligogalactia. Furthermore, they can be also used as the agent for improvement in postoperative nutritional status and/or vasopressor.

When the polypeptide, or the DNA encoding either of them of the present invention is used as a pharmaceutical composition as described above, it can be used by conventional methods. For example, it can be used orally in the form of tablets which may be sugar coated as necessary, capsules, elixirs, microcapsules etc., or non-orally in the form of injectible preparations such as aseptic solutions and suspensions in water or other pharmaceutically acceptable liquids. These preparations can be produced by mixing the polypeptide, a partial peptide thereof, or the DNA encoding either of them with physiologically acceptable carriers, flavoring agents, excipients, vehicles, antiseptics, stabilizers, binders etc. in unit dosage forms required for generally accepted manners of pharmaceutical manufacture. Active ingredient contents in these preparations are set so that an appropriate dose within the specified range is obtained.

Additives which can be mixed in tablets, capsules etc. include binders such as gelatin, corn starch, tragacanth and gum arabic, excipients such as crystalline cellulose, swelling agents such as corn starch, gelatin and alginic acid, lubricants such as magnesium stearate, sweetening agents such as sucrose, lactose and saccharin, and flavoring agents such as peppermint, akamono oil and cherry. When the unit dosage form is a capsule, the above-mentioned materials may further incorporate liquid carriers such as oils and fats. Sterile compositions for injection can be formulated by ordinary methods of pharmaceutical manufacture, for example, by dissolving or suspending active ingredients, naturally occurring vegetable oils such as sesame oil and coconut oil, etc. in vehicles such as water for injection.

Aqueous liquids for injection include physiological saline and isotonic solutions containing glucose and other auxiliary agents, e.g., D-sorbitol, D-mannitol and sodium chloride, and may be used in combination with appropriate dissolution aids such as alcohols, e.g., ethanol, polyalcohols, e.g., propylene glycol and polyethylene glycol, nonionic surfactants, e.g., polysorbate 80 (TM) and HCO-50 etc. oily liquids include sesame oil and soybean oil, and may be used in combination with dissolution aids such as benzyl benzoate and benzyl alcohol. Furthermore the above-mentioned materials may also be formulated with buffers, e.g., phosphate buffer and sodium acetate buffer; soothing agents, e.g., benzalkonium chloride, procaine hydrochloride; stabilizers, e.g., human serum albumin, polyethylene glycol; preservatives, e.g., benzyl alcohol, phenol; antioxidants etc. The thus-prepared injectible liquid is normally filled in an appropriate ampule. Because the thus-obtained preparation is safe and of low toxicity, it can be administered to humans or warm-blooded mammals, e.g., mouse, rats, guinea pig, rabbits, chicken, sheep, pigs, bovines, cats, dogs, monkeys, baboons, chimpanzees, for instance.

The dose of said polypeptide, or the DNA encoding either of them is normally about 0.1-100 mg, preferably 1.0-50 mg, and more preferably 1.0-20 mg per day for an adult (weighing 60 kg) in oral administration, depending on symptoms etc. In non-oral administration, it is advantageous to administer the polypeptide, a partial peptide thereof, or the DNA encoding either of them in the form of injectible preparation at a daily dose of about 0.01-30 mg, preferably about 0.1-20 mg, and more preferably about 0.1-10 mg per administration by an intravenous injection for an adult (weighing 60 kg), depending on subject of administration, target organ, symptoms, method of administration etc. For other animal species, corresponding doses as converted per 60 kg weight can be administered.

The G protein-coupled receptor protein for the above ligand polypeptide of the present invention may be any of G protein-coupled receptor proteins derived from various tissues, e.g. hypophysis, pancreas, brain, kidney, liver, gonad, thyroid gland, gall bladder, bone marrow, adrenal gland, skin, muscle, lung, alimentary canal, blood vessel, heart, etc. of human and other warm-blooded animals, e.g. guinea pig, rat, mouse, swine, sheep, bovine, monkey, etc.; and comprising an amino acid sequence of SEQ ID NO:10, 11, 12, 18 or 14, or a substantial equivalent thereto. Thus, the G protein-coupled receptor protein of the present invention includes, in addition to a protein comprising the SEQ ID NO:10, 11, 12, 13 or 14, those proteins comprising amino acid sequences of about 90-99.9% homology to the amino acid sequence of SEQ ID NO:10, 11, 12, 13 or 14 and having qualitatively substantially equivalent activity to proteins comprising the amino acid sequence of SEQ ID NO:10, 11, 12, 13, or 14. The activities which these proteins are possessed may include ligand binding activity and signal transduction activity. The term “substantially equivalent” means that the nature of the ligand binding activity and the like is equivalent. Therefore, it is allowable that even differences among grades such as the strength of ligand binding activity and the molecular weight of receptor protein are present.

To be further specific, the G protein-coupled receptor proteins include human pituitary-derived G protein-coupled receptor proteins which comprises the amino acid sequence of SEQ ID NO:10 or/and SEQ ID NO:11, mouse pancreas-derived G protein-coupled receptor proteins which comprises the amino acid sequence of SEQ ID NO:13, and mouse pancreas-derived G protein-coupled receptor proteins which comprises the amino acid sequence of SEQ ID NO:14. As the human pituitary-derived G protein-coupled receptor proteins which comprises the amino acid sequence of SEQ ID NO:10 and/or SEQ ID NO:11 include the human pituitary-derived G protein-coupled receptor protein which comprises the amino acid sequence of SEQ ID NO:12.

Here, the protein which comprises an amino acid sequence of SEQ ID NO:12 or a substantial equivalent thereto contains the full-length of the amino acid sequence for human pituitary-derived G protein-coupled receptor protein. The protein which comprises an amino acid sequence of SEQ ID NO:10 or/and SEQ ID NO:11 or a substantial equivalent thereto may be a partial peptide of the protein which comprises an amino acid sequence of SEQ ID NO:12 or a substantial equivalent thereto. The protein which comprises an amino acid sequence of SEQ ID NO:13 or SEQ ID NO:14 or a substantial equivalent thereto is a G protein-coupled receptor protein which is derived from mouse pancreas but, since its amino acid sequence is quite similar to the amino acid sequence of SEQ ID NO:10 or/and SEQ ID NO:11, the protein which comprises an amino acid sequence of SEQ ID NO:13 or 14 or a substantial equivalent thereto is also subsumed in the category of said partial peptide of the protein which comprises an amino acid sequence of SEQ ID NO:12 or a substantial equivalent thereto.

Thus, the above-mentioned protein comprising an amino acid sequence of SEQ ID NO:12 or a substantial equivalent thereto or a partial peptide of the protein or a salt thereof, which will be described below, includes the protein comprising an amino acid sequence of SEQ ID NO:10, 11, 12, or 13 or a substantial equivalent thereto, or a salt thereof.

Furthermore, the G protein-coupled receptor protein includes the protein in which the N-terminal Met has been protected with a protective group, e.g. C₁₋₆ acyl such as formyl or acetyl, the protein in which the N-terminal side of Gln has been cleaved in vivo to form pyroglutamyl, the protein in which the side chain of any relevant constituent amino acid has been protected with a suitable protective group, e.g. C₁₋₆ acyl such as formyl or acetyl, and the complex protein such as glycoproteins available upon attachment of sugar chains.

The salt of G protein-coupled receptor protein includes the same kinds of salts as mentioned for the ligand polypeptide.

The G protein-coupled receptor protein or a salt thereof or a partial peptide thereof can be produced from the tissues or cells of human or other warm-blooded animals by per se known purification techniques or, as described above, by culturing a transformant carrying a DNA coding for the G protein-coupled receptor protein. It can also be produced in accordance with the procedures for peptide synthesis which are described above. The production method is described in, for example, Examples 3, 4, 6 and 17 of WO96/05302, in detail.

The DNA coding for the G protein-coupled receptor protein may be any DNA comprising a nucleotide sequence encoding the G protein-coupled receptor protein which comprises an amino acid sequence of SEQ ID NO:10, 11, 12, 13, or 14 or a substantial equivalent thereto. It may also be any one of genomic DNA, genomic DNA library, tissue- or cell-derived cDNA, tissue- or cell-derived cDNA library, and synthetic DNA. The vector for such a library may include bacteriophage, plasmid, cosmid, and phargimid. Furthermore, using an RNA fraction prepared from a tissue or cells, a direct amplification can be carried out by the RT-PCR method.

To be specific, the DNA encoding the human pituitary-derived G protein-coupled receptor protein which comprises the amino acid sequence of SEQ ID NO:10 include a DNA which comprises the nucleotide sequence of SEQ ID NO:15. The DNA encoding the human pituitary-derived G protein-coupled receptor protein which comprises the amino acid sequence of SEQ ID NO:11 include a DNA which comprises the nucleotide sequence of SEQ ID NO:16. The DNA encoding the human pituitary-derived G protein-coupled receptor protein which comprises the amino acid sequence of SEQ ID NO:12 include a DNA which comprises the nucleotide sequence of SEQ ID NO:17. The DNA encoding the mouse pancreas-derived G protein-coupled receptor protein which comprises the amino acid sequence of SEQ ID NO:13 include a DNA which comprises the nucleotide sequence of SEQ ID NO:18. The DNA encoding the mouse pancreas-derived G protein-coupled receptor protein which comprises the amino acid sequence of SEQ ID NO:14 include a DNA comprising the nucleotide sequence of SEQ ID NO:19.

A method for cloning the DNA completely coding for the G protein-coupled receptor protein, vector, promoter, host cell, a method for transformation, a method for culturing the transformant or a method for separation and purification of the G protein-coupled receptor protein may include the same one as mentioned for the ligand polypeptide.

Described below are uses of the ligand polypeptide of the present invention, the G protein-coupled receptor protein-encoding DNAs and their antibodies.

(1) Prophylactic and Therapeutic Agent for Ligand Polypeptide Deficiency Diseases

The G protein-coupled receptor protein-encoding DNA can be used as a prophylactic and/or therapeutic agent for treating said ligand polypeptide deficiency diseases depending upon the action that said ligand exerts.

For example, when there is a patient for whom the physiological action of the ligand, such as pituitary function modulating action, central nervous system function modulating action or pancreatic function modulating action, cannot be expected because of a decrease in the G protein-coupled receptor protein or ligand polypeptide in vivo, the amount of the G protein-coupled receptor protein or ligand polypeptide in the brain cells of said patient can be increased whereby the action of the ligand can be fully achieved by: (a) administering the G protein-coupled receptor protein-encoding DNA to the patient to express it; or (b) inserting the G protein-coupled receptor protein or ligand polypeptide-encoding DNA into brain cells or the like to said patient. Accordingly, the G protein-coupled receptor protein- or ligand polypeptide-encoding DNA can be used as a safe and less toxic preventive and therapeutic agent for the G protein-coupled receptor protein or ligand polypeptide deficiency diseases.

When the above-mentioned DNA is used as the above-mentioned agent, said DNA may be used alone or after inserting it into a suitable vector such as retrovirus vector, adenovirus vector, adenovirus-associated virus vector, etc. followed by subjecting the product vector to a conventional means which is the same means as using the DNA coding for the ligand polypeptide or partial peptide thereof as the pharmaceutical composition.

(2) Quantitative Determination of the G protein-coupled receptor Protein to the Ligand Polypeptide

The ligand polypeptide that has a binding property for a G protein-coupled receptor protein or a partial peptide thereof, or a salt thereof is capable of determining quantitatively an amount of a G protein-coupled receptor protein or a partial peptide thereof, or a salt thereof in vivo with good sensitivity.

This quantitative determination may be carried out by, for example, combining with a competitive analysis. Thus, a sample to be determined is contacted with the ligand polypeptide so that the concentration of a G protein-coupled receptor protein or a partial peptide thereof in said sample can be determined. In one embodiment of the quantitative determination, the protocols described in the following 1) and 2) or methods similar thereto may be used:

1) Hiroshi Irie (ed): “Radioimmunoassay” (Kodansha, Japan, 1974); and

2) Hiroshi Irie (ed): “Radioimmunoassay, Second Series” (Kodansha, Japan, 1979).

3) Screening of Compound Changing the Binding

Activity of Ligand Polypeptide, or salt thereof (hereinafter sometimes referred to briefly as ligand or ligand polypeptide) with the G protein-coupled receptor Protein

G protein-coupled receptor proteins or partial peptide or salt thereof can be used. Alternatively, expression systems for recombinant G protein-coupled receptor proteins are constructed and receptor binding assay systems using said expression system are used. In these assay systems, it is possible to screen compounds, e.g. peptides, proteins, nonpeptidic compounds, synthetic compounds, fermentation products, cell extracts, animal tissue extracts, etc.; or salts thereof which change the binding activity of a ligand polypeptide with the G protein-coupled receptor protein. Such a compound includes a compound exhibiting a G protein-coupled receptor-mediated cell stimulating activity, e.g. activity of promoting or activity of inhibiting physiological reactions including liberation of arachidonic acid, liberation of acetylchloline, intracellular Ca²⁺ liberation, intracellular cAMP production, intracellular cGMP production, production of inositol phosphate, changes in cell membrane potential, phosphorylation of intracellular proteins activation of c-fos, lowering of pH, activation of G protein, cell promulgation, etc.; so-called “G protein-coupled receptor-agonist”, a compound free from such a cell stimulating activity, so-called “G protein coupled receptor-antagonist”, etc. The term of “change the binding activity of a ligand polypeptide” includes the both concept of the case in which the binding of ligand is inhibited and the case in which the binding of ligand is promoted.

Thus, the present invention provides a method of screening for a compound which changes the binding activity of a ligand with a G protein-coupled receptor protein or a salt thereof, characterized by comparing the following two cases: (i) the case wherein the ligand is contacted with the G protein-coupled receptor protein or salt thereof, or the partial peptide thereof or a salt thereof; and (ii) the case wherein the ligand is contacted with a mixture of the G protein-coupled receptor protein or salt thereof or the partial peptide or salt thereof and said test compound.

In said screening method, one characteristic feature of the present invention resides in that the amount of the ligand bonded with said G protein-coupled receptor protein or the partial peptide thereof, the cell stimulating activity of the ligand, etc. are measured in both the case where (i) the ligand polypeptide is contacted with G protein-coupled receptor proteins or partial peptide thereof and in the case where (ii) the ligand polypeptide and the test compound are contacted with the G protein-coupled receptor protein or the partial peptide thereof, respectively and then compared therebetween.

In one more specific embodiment of the present invention, the following is provided: 1) a method of screening for a compound or a salt thereof which changes the binding activity of a ligand polypeptide with a G protein-coupled receptor protein, characterized in that, when a labeled ligand polypeptide is contacted with a G protein-coupled receptor protein or a partial peptide thereof and when a labeled ligand polypeptide and a test compound are contacted with a G protein-coupled receptor protein or a partial peptide thereof, the amounts of the labeled ligand polypeptide bonded with said protein or a partial peptide thereof or a salt thereof are measured and compared; 2) a method of screening for a compound or a salt thereof which changes the binding activity of a ligand polypeptide with a G protein-coupled receptor protein, characterized in that, when a labeled ligand polypeptide is contacted with cells containing G protein-coupled receptor proteins or a membrane fraction of said cells and when a labeled ligand polypeptide and a test compound are contacted with cells containing G protein-coupled receptor proteins or a membrane fraction of said cells, the amounts of the labeled ligand polypeptide binding with said protein or a partial peptide thereof or a salt thereof are measured and compared; 3) a method of screening for a compound or a salt thereof which changes the binding activity of a ligand polypeptide with a G protein-coupled receptor protein, characterized in that, when a labeled ligand polypeptide is contacted with G protein-coupled receptor proteins expressed on the cell membrane by culturing a transformant carrying a G protein-coupled receptor protein-encoding DNA and when a labeled ligand polypeptide and a test compound are contacted with G protein-coupled receptor proteins expressed on the cell membrane by culturing a transformant carrying a G protein-coupled receptor protein-encoding DNA, the amounts of the labeled ligand polypeptide binding with said G protein-coupled receptor protein are measured and compared; 4) a method of screening for a compound or a salt thereof which changes the binding of a ligand polypeptide with a G protein-coupled receptor protein, characterized in that, when a G protein-coupled receptor protein-activating compound, e.g. a ligand polypeptide of the present invention, etc. is contacted with cells containing G protein-coupled receptor proteins and when the G protein-coupled receptor protein-activating compound and a test compound are contacted with cells containing G protein-coupled receptor proteins, the resulting G protein-coupled receptor protein-mediated cell stimulating activities, e.g. activities of promoting or activities of inhibiting physiological responses including liberation of arachidonic acid, liberation of acetylcholine, intracellular Ca2+ liberation, intracellular cAMP production, intracellular cGMP production, production of inositol phosphate, changes in cell membrane potential, phosphorylation of intracellular proteins, activation of c-fos, lowering of pH, activation of G protein, cell promulgation, etc.; are measured and compared; and 5) a method of screening for a compound or a salt thereof which changes the binding activity of a ligand polypeptide with a G protein-coupled receptor protein, characterized in that, when a G protein-coupled receptor protein-activating compound, e.g. a ligand polypeptide of the present invention, etc. is contacted with G protein-coupled receptor proteins expressed on cell membranes by culturing transformants carrying G protein-coupled receptor protein-encoding DNA and when a G protein-coupled receptor protein-activating compound and a test compound are contacted with the G protein-coupled receptor protein expressed on the cell membrane by culturing the transformant carrying the G protein-coupled receptor protein-encoding DNA, the resulting G protein-coupled receptor protein-mediated cell stimulating activities, e.g. activities of promoting or activities of inhibiting physiological responses such as liberation of arachidonic acid, liberation of acetylcholine, intracellular Ca2+ liberation, intracellular cAMP production, intracellular cGMP production, production of inositol phosphate, changes in cell membrane potential, phosphorylation of intracellular proteins, activation of c-fos, lowering of pH, activation of G protein, and cell promulgation, etc.; are measured and compared.

The G protein-coupled receptor agonist or antagonist can be screened by, first, obtaining a candidate compound by using G protein-coupled receptor protein-containing cells, tissues or cell membrane fractions derived from mouse, rat or the like (primary screening), then, making sure whether the candidate compound really inhibits the binding between human G protein-coupled receptor proteins and ligands (secondary screening). Other receptor proteins inevitably exist and when the cells, the tissues or the cell membrane fractions are used, they intrinsically make it difficult to screen agonists or antagonists to the desired receptor proteins. By using the human-derived G protein-coupled receptor protein, however, there is no need of effecting the primary screening, whereby it is possible to efficiently screen a compound that changes the binding activity between a ligand and a G protein-coupled receptor. Additionally, it is possible to evaluate whether the compound that is screened is a G protein-coupled receptor agonist or a G protein-coupled receptor antagonist.

Specific explanations of the screening method will be given as hereunder.

First, with respect to the G protein-coupled receptor protein used for the screening method of the present invention, any product may be used so far as it contains G protein-coupled receptor proteins or partial peptides thereof although the use of a membrane fraction of mammalian organs is preferable. However, human organs can be extremely scarce and, accordingly, G protein-coupled receptor proteins which are expressed in a large amount using a recombinant technique are suitable for the screening.

In the manufacture of the G protein-coupled receptor protein, the above-mentioned method can be used.

When the G protein-coupled receptor protein-containing cells or cell membrane fractions are used in the screening method, the above-mentioned method can be used.

In conducting the above-mentioned methods 1) to 3) for screening the compound capable of changing the binding activity of the ligand with the G protein-coupled receptor protein, a suitable G protein-coupled receptor fraction and a labeled ligand polypeptide are necessary. With respect to the G protein-coupled receptor fraction, it is preferred to use naturally occurring G protein-coupled receptors (natural type G protein-coupled receptors) or recombinant type G protein-coupled receptor fractions with the activity equivalent to that of the natural type G protein coupled reaction. Here the term “activity equivalent to” means the same ligand binding activity, or the substantially equivalent ligand binding activity.

With respect to the labeled ligand, it is possible to use labeled ligands, labeled ligand amalogized compounds, etc. For example, ligands labeled with [³H], [¹²⁵I], [¹⁴C], [³⁵S], etc. and other labeled substances may be utilized.

Specifically, G protein-coupled receptor protein-containing cells or cell membrane fractions are first suspended in a buffer which is suitable for the determining method to prepare the receptor sample in conducting the screening for a compound which changes the binding activity of the ligand with the G protein-coupled receptor protein. With respect to the buffer, any buffer such as Tris-HCl buffer or phosphate buffer of pH 4-10, preferably, pH 6-8 which does not inhibit the binding of the ligand with the receptor may be used.

In addition, a surface-active agent such as CHAPS, Tween 80TM (Kao-Atlas, Japan), digitonin, deoxycholate, etc. and/or various proteins such as bovine serum albumin (BSA), gelatin, etc. may be added to the buffer with an object of decreasing the nonspecific binding. Further, a protease inhibitor such as PMSF, leupeptin, E-64 manufactured by Peptide Laboratory, Japan, pepstatin, etc. may be added with an object of inhibiting the decomposition of the receptor and the ligand by protease. A labeled ligand in a certain amount (5,000 cpm to 500,000 cpm) is added to 0.01 ml to 10 ml of said receptor solution and, at the same time, 10-4M to 10-10M of a test compound coexists. In order to determine the nonspecific binding amount (NSB), a reaction tube to which a great excessive amount of an unlabeled test compounds is added is prepared as well.

The reaction is carried out at 0-50 C., preferably at 4-37 C. for 20 minutes to 24 hours, preferably 30 minutes to three hours. After the reaction, it is filtered through a glass fiber filter, a filter paper, or the like, washed with a suitable amount of the same buffer and the radioactivity retained in the glass fiber filter, etc. is measured by means of a liquid scintillation counter of a gamma-counter. Supposing that the count (B₀−NSB) obtained by subtracting the nonspecific binding amount (NSB) from the total binding amount (B₀) wherein an antagonizing substance is not present is set at 100%, a test compound in which the specific binding amount (B−NSB) obtained by subtracting the nonspecific binding amount (NSB) from the total binding amount (B) is, for example, less than 50% may be selected as a candidate ligand to the G protein-coupled receptor protein of the present invention.

In conducting the above-mentioned methods 4) to 5) for screening the compound which changes the binding activity of the ligand with the G protein-coupled receptor protein, the G protein-coupled receptor protein-mediated cell stimulating activity, e.g. activities of promoting or activities of inhibiting physiological responses such as release of arachidonic acid, release of acetylcholine, intracellular Ca²⁺ increase, intracellular cAMP production, production of inositol phosphate, changes in the cell membrane potential, phosphorylation of intracellular proteins, activation of c-fos, lowering of pH, activation of G protein and cell proliferation, etc.; may be measured by known methods or by the use of commercially available measuring kits. To be more specific, G protein-coupled receptor protein-containing cells are at first cultured in a multiwell plate or the like.

In conducting the screening, it is substituted with a suitable buffer which does not show toxicity to fresh media or cells in advance, incubated under appropriate conditions and for a specified time after adding a test compound, etc. thereto. The resultant cells are extracted or the supernatant liquid is recovered and the resulting product is determined, preferably quantitatively, by each of the methods. When it is difficult to identify the production of the indicative substance, e.g. arachidonic acid, etc. which is to be an indication for the cell stimulating activity due to the presence of decomposing enzymes contained in the cell, an assay may be carried out by adding an inhibitor against said decomposing enzyme. With respect to the activities such as an inhibitory action against cAMP production, it may be detected as an inhibitory action against the cAMP production in the cells whose fundamental production has been increased by forskolin or the like.

In conducting a screening by measuring the cell stimulating activity, cells in which a suitable G protein-coupled receptor protein is expressed are necessary. Preferred G protein-coupled receptor protein-expressing cells are naturally occurring G protein-coupled receptor protein (natural type G protein-coupled receptor protein)-containing cell lines or strains, e.g. mouse pancreatic β cell line, MIN6, etc., the above-mentioned recombinant type G protein-coupled receptor protein-expressing cell lines or strains, etc.

Examples of the test compound includes peptide, proteins, non-peptidic compounds, synthesized compounds, fermentation products, cell extracts, plant extracts, animal tissue extracts, serum, blood, body fluid, etc. Those compounds may be novel or known.

A kit for screening the compound which changes the binding activity of the ligand with the G protein-coupled receptor protein or a salt thereof comprises a G protein-coupled receptor protein or a partial peptide thereof, or G protein-coupled receptor protein-containing cells or cell membrane fraction thereof.

Examples of the screening kit include as follows: 1. Reagent for Determining Ligand. 1) Buffer for Measurement and Buffer for Washing.

The product wherein 0.05% of bovine serum albumin (manufactured by Sigma) is added to Hanks' Balanced Salt Solution (manufactured by Gibco).

This may be sterilized by filtration through a membrane filter with a 0.45 μm pore size, and stored at 4 C. or may be prepared upon use. 2) Sample of G protein-coupled receptor Protein.

CHO cells in which a G protein-coupled receptor protein is expressed are subcultured at the rate of 5×105 cells/well in a 12-well plate and cultured at 37 C. with a 5% CO₂ and 95% air atmosphere for two days to prepare the sample. 3) Labeled Ligand.

The ligand which is labeled with commercially available [³H], [¹²⁵I], [¹⁴C], [35S], etc.

The product in a state of an aqueous solution is stored at 4 C. or at −20 C. and, upon use, diluted to 1 μM with a buffer for the measurement. 4) Standard Ligand Solution.

Ligand is dissolved in PBS containing 0.1% of bovine serum albumin (manufactured by Sigma) to make 1 mM and stored at −20 C.

2. Method of the Measurement. 1) CHO cells are cultured in a 12-well tissue culture plate to express G protein-coupled receptor proteins. The G protein-coupled receptor protein-expressing CHO cells are washed with 1 ml of buffer for the measurement twice. Then 490 μl of buffer for the measurement is added to each well. 2) Five μl of a test compound solution of 10⁻³ to 10⁻¹⁰ M is added, then 5 μl of a labeled ligand is added and subjected to reaction at room temperature for one hour. For knowing the non-specific binding amount, 5 μl of the ligand of 10⁻³ M is added instead of the test compound. 3) The reaction solution is removed from the well, which is washed with 1 ml of buffer for the measurement three times. The labeled ligand binding with the cells is dissolved in 0.2N NaOH-1% SDS and mixed with 4 ml of a liquid scintillator A (such as manufactured by Wako Pure Chemical, Japan). 4) Radioactivity is measured using a liquid scintillation counter (e.g., one manufactured by Beckmann) and PMB (percent maximum binding) is calculated by the following equation:

PMB=[(B-NSB)/(B₀- NSB)]×100 PMB:

Percent maximum binding B: Value when a sample is added NSB: Nonspecific binding B₀: Maximum binding

The compound or a salt thereof obtained by the screening method or by the screening kit is a compound which changes the binding activity of a ligand polypeptide with a G protein-coupled receptor protein, wherein the compound inhibits or promotes the binding, and, more particularly, it is a compound having a cell stimulating activity mediated via a G protein-coupled receptor or a salt thereof, so-called “G protein-coupled receptor agonist” or a compound having no said stimulating activity, so-called “G protein-coupled receptor antagonist”. Examples of said compound are peptides, proteins, non-peptidic compounds, synthesized compounds, fermentation products, etc. and the compound may be novel or known.

Said G protein coupled receptor agonist has the same physiological action as the ligand to the G protein-coupled receptor protein and, therefore, it is useful as a safe and less toxic pharmaceutical composition depending upon said ligand activity.

On the other hand, said G protein-coupled receptor antagonist is capable of inhibiting the physiological activity of the ligand to the G protein-coupled receptor protein and, therefore, it is useful as a safe and less toxic pharmaceutical composition for inhibiting said ligand activity.

The ligand polypeptide of the present invention relates to the pituitary function modulating action, central nervous system function modulating action or pancreatic function modulating action. Therefore, the above-mentioned agonist or antagonist can be used as a therapeutic and/or prophylactic agent for dementia such as senile dementia, cerebrovascular dementia (dementia due to cerebrovascular disorder), dementia associated with phylodegenerative retroplastic diseases (e.g. Alzheimer's disease, Parkinson's disease, Pick's disease, Huntington's disease, etc.), dementia due to infectious diseases (e.g. delayed viral infections such as Creutzfelt-Jakob disease), dementia associated with endocrine, metabolic, and toxic diseases (e.g. hypothyroidism, vitamin B12 deficiency, alcoholism, and poisoning due to various drugs, metals, or organic compounds), dementia associated with oncogenic diseases (e.g. brain tumor), dementia due to traumatic diseases (e.g. chronic subdural hematoma):, depression (melancholia), hyperkinetic (microencephalopathy) syndrome, disturbance of consciousness, anxiety syndrome, schizophrenia, horror, growth hormone secretory disease (e.g. gigantism, acromegalic gigantism etc.), hyperphagia, polyphagia, hypercholesterolemia, hyperglyceridemia, hyperlipemia, hyperprolactinemia, hypoglycemia, pituitarism, pituitary dwarfism, diabetes (e.g. diabetic complications, diabetic nephropathy, diabetic neuropathy, diabetic retinopathy etc.), cancer (e.g. mammary cancer, lymphatic leukemia, cystic cancer, ovary cancer, prostatic cancer etc.), pancreatitis, renal disease (e.g. chromic renal failure, nephritis etc.), Turner's syndrome, neurosis, rheumatoid arthritis, spinal injury, transient brain ischemia, amyotrophic lateral sclerosis, acute myocardial infarction, spinocerebellar degeneration, bone fracture, trauma, atopic dermatitis, osteoporosis, asthma, epilepsy, infertility or oligogalactia. Furthermore, the agonist or antagonist can be also used as hypnotic-sedative agent for improvement in postoperative nutritional status, vasopressor or depressor.

When the compound or the salt thereof obtained by the screening method or by the screening kit is used as the pharmaceutical composition, a conventional means which is the same means as using above-mentioned ligand polypeptide as the pharmaceutical composition may be applied therefor.

(4) Manufacture of Antibody or Antiserum against the Ligand Polypeptide of the present invention

Antibodies, e.g. polyclonal antibody, monoclonal antibody, and antisera against the ligand polypeptide may be manufactured by antibody- or antiserum-manufacturing methods per se known to those of skill in the art or methods similar thereto, using the ligand polypeptide as antigen. For example, polyclonal antibodies can be manufactured by the method as given below.

### Preparation of a Polyclonal Antibody

The above-mentioned polypeptide or protein as the antigen is coupled to a carrier protein. The carrier protein may for example be bovine thyroglobulin, bovine serum albumin, bovine gamma-globulin, hemocyanine, or Freund's complete adjuvant (Difco).

The coupling reaction between the antigen protein and the carrier protein can be carried out by the known procedure. The reagent for use in the coupling reaction includes but is not limited to glutaraldehyde and water-soluble carbodiimide. The suitable ratio of the antigen protein to the carrier protein is about 1:1 through about 1:10 and as to the reaction pH, satisfactory results are obtained in many cases when the reaction is carried out around neutral, particularly in the range of pH about 6-8. The reaction time is preferably about 1 to 12 hours in many cases and more preferably about 2 to 6 hours. The conjugate thus obtained is dialyzed against water at about 0 to 18 C. in the routine manner and stored frozen or optionally lyophilized and stored.

For the production of a polyclonal antibody, a warm-blooded animal is inoculated with the immunogen produced in the manner described hereinbefore. The warm-blooded animal that can be used for this purpose includes mammalian warm-blooded animals, e.g. rabbit, sheep, goat, rat, mouse, guinea pig, bovine, equine, swine, etc.; and avian species, e.g. chicken, dove, duck, goose, quail, etc. Regarding the methodology for inoculating a warm-blooded animal with the immunogen, the innoculum size of the immunogen may be just sufficient for antibody production. For example, the desired antibody can be produced in many instances by emulsifying 1 mg of the immunogen in 1 ml of saline with Freund's complete adjuvant and injecting the emulsion subcutaneously at the back and hind-limb footpad of rabbits 5 times at 4-week intervals. For harvesting the antibody produced in the warm-blooded animal, for example a rabbit, the blood is withdrawn from the auricular vein usually during day 7 through day 12 after the last inoculation dose and centrifuged to recover an antiserum. For purification, the antiserum is generally subjected to affinity chromatography using a carrier to which each antigen peptide has been conjugated and the adsorbed fraction is recovered to provide a polyclonal antibody.

The monoclonal antibody can be produced by the following method.

### Preparation of Monoclonal Antibody

(a) Preparation of Monoclonal Antibody-Producing Cells.

The ligand polypeptide is administered to warm-blooded animals either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once every two to six weeks and two to ten times in total. Examples of the applicable warm-blooded animals are monkeys, rabbits, dogs, guinea pigs, mice, rats, sheep, goats and chickens and the use of mice and rats is preferred.

In the preparation of the cells which produce monoclonal antibodies, an animal wherein the antibody titer is noted is selected from warm-blooded animals (e.g. mice) immunized with antigens, then spleen or lymph node is collected after two to five days from the final immunization and antibody-producing cells contained therein are fused with myeloma cells to give monoclonal antibody-producing hybridomas. Measurement of the antibody titer in antisera may, for example, be carried out by reacting a labeled ligand polypeptide or a labeled G protein-coupled receptor protein (which will be mentioned later) with the antiserum followed by measuring the binding activity of the labeling agent with the antibody. The operation for fusing may be carried out, for example, by the method of Koehler and Milstein (Nature, 256, 495, 1975). Examples of the fusion accelerator are polyethylene glycol (PEG), Sendai virus, etc. and the use of PEG is preferred.

Examples of the myeloma cells are NS-1, P3U1, SP2/0, AP-1, etc. and the use of P3U1 is preferred. The preferred fusion ratio of the numbers of antibody-producing cells used (spleen cells) to the numbers of myeloma cells is within a range of about 1:1 to 20:1. When PEG (preferably, PEG 1000 to PEG 6000) is added in a concentration of about 10-80% followed by incubating at 20-40 C. (preferably, at 30-37 C.) for one to ten minutes, an efficient cell fusion can be carried out.

Various methods may be applied for screening a hybridoma which produces anti-ligand polypeptide antibody. For example, a supernatant liquid of hybridoma culture is added to a solid phase (e.g. microplate) to which the ligand polypeptide antigen is adsorbed either directly or with a carrier, then anti-immunoglobulin antibody (anti-mouse immunoglobulin antibody is used when the cells used for the cell fusion are those of mouse) which is labeled with a radioactive substance, an enzyme or the like, or protein A is added thereto and then anti-ligand polypeptide monoclonal antibodies bound on the solid phase are detected; or a supernatant liquid of the hybridoma culture is added to the solid phase to which anti-immunoglobulin or protein A is adsorbed, then the ligand polypeptide labeled with a radioactive substance or an enzyme is added and anti-ligand polypeptide monoclonal antibodies bonded with the solid phase is detected.

Selection and cloning of the anti-ligand polypeptide monoclonal antibody-producing hybridoma may be carried out by methods per se known to those of skill in the art or methods similar thereto. Usually, it is carried out in a medium for animal cells, containing HAT (hypoxanthine, aminopterin and thymidine). With respect to a medium for the selection, for the cloning and for the growth, any medium may be used so far as hybridoma is able to grow therein. Examples of the medium are an RPMI 1640 medium (Dainippon Pharmaceutical Co., Ltd., Japan) containing 1-20% (preferably 10-20%) of fetal calf serum (FCS), a GIT medium (Wako Pure Chemical, Japan) containing 1-20% of fetal calf serum and a serum-free medium for hybridoma culturing (SFM-101; Nissui Seiyaku, Japan). The culturing temperature is usually 20-40 C. and, preferably, about 37 C. The culturing time is usually from five days to three weeks and, preferably, one to two weeks. The culturing is usually carried out in 5% carbon dioxide gas. The antibody titer of the supernatant liquid of the hybridoma culture may be measured in the same manner as the above-mentioned measurement of the antibody titer of the anti-ligand polypeptide in the antiserum. (b) Purification of the Monoclonal Antibody.

In the same manner as the separation/purification of conventional polyclonal antibodies, the separation/purification of the anti-ligand polypeptide monoclonal antibody may be carried out by methods for separating/purifying immunoglobulin such as salting-out, precipitation with an alcohol, isoelectric precipitation, electrophoresis, adsorption/deadsorption using ion exchangers such as DEAE, ultracentrifugation, gel filtration, specific purifying methods in which only an antibody is collected by treatment with an active adsorbent such as an antigen-binding solid phase, protein A or protein G and the bond is dissociated whereupon the antibody is obtained.

The ligand polypeptide antibody which is manufactured by the aforementioned method (a) or (b) is capable of specifically recognizing ligand polypeptide, accordingly, it can be used for a quantitative determination of the ligand polypeptide in test liquid samples and particularly for a quantitative determination by sandwich immunoassays.

Thus, the present invention provides, for example, the following methods: (i) a quantitative determination of a ligand polypeptide in a test liquid sample, which comprises (a) competitively reacting the test liquid sample and a labeled ligand polypeptide with an antibody which reacts with the ligand polypeptide, and (b) measuring the ratio of the labeled ligand polypeptide binding with said antibody; and (ii) a quantitative determination of a ligand polypeptide in a test liquid sample, which comprises (a) reacting the test liquid sample with an antibody immobilized on an insoluble carrier and a labeled antibody simultaneously or continuously, and (b) measuring the activity of the labeling agent on the insoluble carrier wherein one antibody is capable of recognizing the N-terminal region of the ligand polypeptide while another antibody is capable of recognizing the C-terminal region of the ligand polypeptide.

When the monoclonal antibody of the present invention recognizing a ligand polypeptide (hereinafter, may be referred to as “anti-ligand polypeptide antibody”) is used, ligand polypeptide can be measured and, moreover, can be detected by means of a tissue staining, etc. as well. For such an object, antibody molecules per se may be used or F(ab′)2′ Fab′ or Fab fractions of the antibody molecule may be used too. There is no particular limitation for the measuring method using the antibody of the present invention and any measuring method may be used so far as it relates to a method in which the amount of antibody, antigen or antibody-antigen complex, depending on or corresponding to the amount of antigen, e.g. the amount of ligand polypeptide, etc. in the liquid sample to be measured, is detected by a chemical or a physical means and then calculated using a standard curve prepared by a standard solution containing the known amount of antigen. For example, nephrometry, competitive method, immunometric method and sandwich method are suitably used and, in terms of sensitivity and specificity, the sandwich method which will be described herein later is particularly preferred.

Examples of the labeling agent used in the measuring method using the labeling substance are radioisotopes, enzymes, fluorescent substances, luminescent substances, colloids, magnetic substances, etc. Examples of the radioisotope are [¹²⁵I], [¹³¹I], [³H] and [¹⁴C]; preferred examples of the enzyme are those which are stable and with big specific activity, such as β-galactosidase, β-glucosidase, alkali phosphatase, peroxidase and malate dehydrogenase; examples of the fluorescent substance are fluorescamine, fluorescein isothiocyanate, etc.; and examples of the luminescent substance are luminol, luminol derivatives, luciferin, lucigenin, etc. Further, a biotin-avidin system may also be used for binding an antibody or antigen with a labeling agent.

In an insolubilization (immobilization) of antigens or antibodies, a physical adsorption may be used or a chemical binding which is usually used for insolubilization or immobilization of proteins or enzymes may be used as well. Examples of the carrier are insoluble polysaccharides such as agarose, dextran and cellulose; synthetic resins such as polystyrene, polyacrylamide and silicone; glass; etc.

In a sandwich (or two-site) method, the test liquid is subjected to reaction with an insolubilized anti-ligand polypeptide antibody (the first reaction), then it is subjected to reaction with a labeled anti-ligand polypeptide antibody (the second reaction) and the activity of the labeling agent on the insoluble carrier is measured whereupon the amount of the ligand polypeptide in the test liquid can be determined. The first reaction and the second reaction may be conducted reversely or simultaneously or they may be conducted with an interval. The type of the labeling agent and the method of insolubilization (immobilization) may be the same as those mentioned already herein. In the immunoassay by means of a sandwich method, it is not always necessary that the antibody used for the labeled antibody and the antibody for the solid phase is one type or one species but, with an object of improving the measuring sensitivity, etc., a mixture of two or more antibodies may be used too.

In the method of measuring ligand polypeptide by the sandwich method of the present invention, the preferred anti-ligand polypeptide antibodies used for the first and the second reactions are antibodies wherein their sites binding to the ligand polypeptide are different each other. Thus, the antibodies used in the first and the second reactions are those wherein, when the antibody used in the second reaction recognizes the C-terminal region of the ligand polypeptide, then the antibody recognizing the site other than C-terminal regions, e.g. recognizing the N-terminal region, is preferably used in the first reaction.

The anti-ligand polypeptide antibody of the present invention may be used in a measuring system other than the sandwich method such as a competitive method, an immunometric method and a nephrometry. In a competitive method, an antigen in the test solution and a labeled antigen are subjected to reaction with an antibody in a competitive manner, then an unreacted labeled antigen (F) and a labeled antigen binding with an antibody (B) are separated (i.e. B/F separation) and the labeled amount of any of B and F is measured whereupon the amount of the antigen in the test solution is determined. With respect to a method for such a reaction, there are a liquid phase method in which a soluble antibody is used as the antibody and the B/F separation is conducted by polyethylene glycol, a second antibody to the above-mentioned antibody, etc.; and a solid phase method in which an immobilized antibody is used as the first antibody or a soluble antibody is used as the first antibody while an immobilized antibody is used as the second antibody.

In an immunometric method, an antigen in the test solution and an immobilized antigen are subjected to a competitive reaction with a certain amount of a labeled antibody followed by separating into solid and liquid phases; or the antigen in the test solution and an excess amount of labeled antibody are reacted, then a immobilized antigen is added to bind an unreacted labeled antibody with the solid phase and separated into solid and liquid phases. After that, the labeled amount of any of the phases is measured to determine the antigen amount in the test solution.

In a nephrometry, the amount of insoluble sediment which is produced as a result of the antigen-antibody reaction in a gel or in a solution is measured. Even when the antigen amount in the test solution is small and only a small amount of the sediment is obtained, a laser nephrometry wherein scattering of laser is utilized can be suitably used.

In applying each of those immunological measuring methods (immunoassays) to the measuring method of the present invention, it is not necessary to set up any special condition, operation, etc. therefor. A measuring system (assay system) for ligand polypeptide may be constructed taking the technical consideration of the persons skilled in the art into consideration in the conventional conditions and operations for each of the methods. With details of those conventional technical means, a variety of reviews, reference books, etc. may be referred to. They are, for example, Hiroshi Irie (ed): “Radioimmunoassay” (Kodansha, Japan, 1974); Hiroshi Irie (ed): “Radioimmunoassay; Second Series” (Kodansha, Japan, 1979); Eiji Ishikawa et al. (ed): “Enzyme Immunoassay” (Igaku Shoin, Japan, 1978); Eiji Ishikawa et al. (ed): “Enzyme Immunoassay” (Second Edition) (Igaku Shoin, Japan, 1982); Eiji Ishikawa et al. (ed): “Enzyme Immunoassay” (Third Edition) (Igaku Shoin, Japan, 1987); “Methods in Enzymology” Vol. 70 (Immunochemical Techniques (Part A)); ibid. Vo. 73 (Immunochemical Techniques (Part B)); ibid. Vo. 74 (Immunochemical Techniques (Part C)); ibid. Vo. 84 (Immunochemical Techniques (Part D: Selected Immunoassays)); ibid. Vol. 92 (Immunochemical Techniques (Part E: Monoclonal Antibodies and General Immunoassay Methods)); ibid. Vol. 121 (Immunochemical Techniques (Part I: Hybridoma Technology and Monoclonal Antibodies)) (Academic Press); etc.

As such, the amount of ligand polypeptide can now be determined with a high precision using the anti-ligand polypeptide antibody of the present invention. (5) Construction of a non-human transgenic animal

The present invention further provides a non-human mammal harboring a foreign DNA coding for the ligand polypeptide of the present invention (hereinafter referred to briefly as foreign DNA) or a mutant thereof (sometimes referred to briefly as a foreign mutant DNA).

Thus, the present invention provides (1) a non-human mammal harboring a foreign DNA of the present invention or a foreign mutant DNA thereof; (2) the non-human mammal according to (1) which is a rodent; (3) the non-human mammalian according to (2) wherein the rodent is a mouse or rat; (4) a recombinant vector containing the foreign DNA of the present invention or a foreign mutant DNA thereof and capable of being expressed in a mammal; and (5) a pharmaceutical composition for gene theraphy which comprises the recombinant vector according to (4).

Examples of the DNA coding for the ligand polypeptide of the present invention is a DNA comprises the DNA having a nucleotide sequence encoding the polypeptide comprising the amino acid sequence represented by SEQ ID NO:1, or a substantial equivalent thereof (hereinafter, may simply referred as the DNA of the present invention), such as the DNA comprising the nucleotide sequence represented by SEQ ID NO:2 or SEQ ID NO:3.

The non-human mammal harboring the foreign DNA of the present invention or a foreign mutant DNA thereof (hereinafter referred to briefly as the transgenic animal of the present invention) can be constructed by transferring the objective DNA to a germinal cell such as an unfertilized egg cell, fertilized egg cell, or sperm cell or its primordial cell, preferably in the period of embryogenesis in the ontogenesis of a non-human mammal (more preferably in the stage of a single cell or a fertilized egg cell and generally at the 8-cell stage or earlier), by the calcium phosphate method, electric pulse method, lipofection method, agglutination method, microinjection method, particle gun method, or DEAE-dextran method.

Further, by using above-mentioned transferring method, the DNA of the present invention can be introduced into somatic cells, various organs in the body, tissue cells and can be used for cell cultivation, tissue cultivation, etc. Moreover, these cells can be hybridized with above-mentioned germinal cell to produce the non-human transgenic mammal.

The non-human mammal used includes bovine, swine, sheep, goat, rabbit, canine, feline, guinea pig, hamster, murine, rat, and so on. From the standpoint of construction of a diseased animal model, rodents which have comparatively short ontogenesis and life cycles and can be easily bred, particularly mice (e.g. pure strains such as C57BL/6, DBA2, etc. and hybrid strains such as B6C3F1, BDF1, B6D2F1, BALB/c, ICR, etc.) or rats (e.g. Wistar, S. Dak., etc.) are preferred.

The “mammal” as mentioned with reference to the recombinant vector capable of being expressed in a mammal includes the same non-human mammals as those mentioned above and humans.

The foreign DNA of the present invention may be one derived from a mammal of the same species as the host animal or a mammal of a different species. For transfer of the DNA of the present invention to the host animal, it is generally advantageous to use a DNA construct prepared by linking the DNA at downstream of a promoter capable of being expressed in animal cells. For example, in transferring the human-derived DNA of the present invention, this human DNA of the present invention can be linked at downstream of a promoter capable of causing expression of DNAs derived from various animals (e.g. rabbit, canine, feline, guinea pig, hamster, rat, murine, etc.) harboring the DNA of the present invention having high homology thereto to prepare a DNA construct (e.g. a vector) which can then be microinjected into a fertilized egg cell of a host mammal such as a fertilized murine egg cell, whereby a transgenic mammal showing a high expression of the DNA of the present invention can be provided.

Examples of the expression vector used for the protein of the present invention are plasmids derived from E. coli, plasmids derived from B. subtilis, plasmids of the yeast origin, γ phage and other bacteriophages, retroviruses such as Molony leukemia virus, and animal viruses such as vaccinia virus and vaculovirus. Preferable examples are plasmids of the E. coli origin, plasmids of the B. subtilis origin, and yeast-derived plasmids.

The promoter for the regulation of the expression of the DNA are (1) promoters for DNAs derived from viruses (e.g. simian virus, cytomegalovirus, Molony leukemia virus, JC virus, papilloma virus, poliovirus, etc.), (2) promoters derived from mammals (e.g. man, rabbit, dog, cat, guinea pig, hamster, rat, mouse, etc.) for albumin, insulin II, uroprakin II, elastase, erythropoietin, endothelin, muscle creatine kinase, glial fibrillary acidic protein, glutathione S-transferase, platelet-derived growth factor β, keratin K1, K10, and K14, collagen type I and type II, cyclic AMP-dependent protein kinase βI subunit, dystrophin, tartaric acid-resistant alkaline phosphatase, atrial natriuretic factor, endothelial receptor tyrosine kinase (generally abbreviated as Tie2), sodium/potassium-exchanging adenosinetriphosphatase (Na+, K+-ATPase), neurofilament light chain, metallothionein I and IIA, metalloprotease I tissue inhibitor, MHC Class I antigen (H-2L), H-ras, renin, dopamine β-hydroxylase, thyroid peroxidase (TPO), poly-peptide chain elongation factor 1α (EF-1α), β actin, α- and β-myosin heavy chain, myosin light chains 1 and 2, myelin basic protein, thyroglobulin, Thy-1, immunoglobulin H chain variable region (VNP), serum amyloid P component, myoglobin, troponin C, smooth muscle α-actin, preproenkephalin A or vasopressin, and so on. Preferable promoters are promoters conducive to high expression in the whole body, such as cytomegalovirus promoter, human polypeptide chain elongation factor 1α (EF-1α) promoter, and human and chicken β-actin promoters.

The vector preferably has a sequence for terminating the transcription of the objective mRNA (generally called terminator) in the transgenic mammal. The examples of the sequence are sequences derived from viruses, various mammals. Preferable examples are the SV40 terminator derived from simian virus, and so on.

In addition, for enhancing the expression of the objective DNA, it is possible, depending on the specific objective, to link the splicing signal, enhancer domain, a portion of the eucaryotic DNA intron, etc. at upstream of the 5′-end of the promoter region, between the promoter region and the translated region, or at downstream of the 3′-end of the translated region.

The translation region of the normal protein of the present invention can be obtained, as the whole or part of the genomic DNA, from the DNAs derived from the liver, kidney, or thyroid cells or fibroblasts of various mammals (e.g. rabbit, canine, feline, guinea pig, hamster, rat, murine, man, etc.) or from various commercial genomic DNA libraries, or starting with the complementary DNAs prepared from RNAs derived from the liver, kidney, thyroid cells or fibroblasts by the known technique. The foreign abnormal DNA can be constructed by mutating the translated region of the normal protein obtained from the above-mentioned cells or tissues by the mutagenesis method.

The translated region can be prepared as a DNA construct which can be expressed in a transgenic animal, by the routine recombinant DNA technique, i.e. by coupling it at downstream of the promoter and, if desired, at upstream of the transcription termination site.

The transfer of the DNA of the present invention at the fertilized egg cell stage insures that the DNA will be ubiquitous in all the germ cells and somatic cells of the host mammal. The presence of the DNA of the present invention in the germ cells of the transgenic animal following DNA transfer means that all the germ cells and somatic cells of all the progeny of the transgenic animal harbor the DNA of the present invention. Thus, the offspring of animals of this line to which DNA is passed down have the DNA of the present invention in their germ cells and somatic cells.

The non-human mammal to which the foreign normal DNA of the present invention has been transferred can be verified by mating to retain the DNA stably and then bred as a strain harboring the transferred DNA from generation to generation under the usual breeding conditions. The transfer of the DNA of the present invention in the fertilized egg cell stage is carried out in such a manner that the transferred DNA will be present in excess in all the germ cells and somatic cells of the transgenic animal. The presence of an excess of the DNA of the present invention in the germ cells of the transgenic animal means that all the progeny of this line harbor an excess of the DNA of the present invention in their germ cells and somatic cells. By preparing homozygous animals having the transferred DNA in both homologous chromosomes and mating the animals of both sexes, they can be bred serially so that all the progeny may harbor an excess of the DNA.

The non-human mammal harboring the normal DNA of the present invention features a high expression of the DNA and may eventually develop a hyperergasia of the protein of the present invention through activation of the function of the endogenous normal DNA and, therefore, can be utilized as an animal model of the disease. For example, by using the transgenic animal harboring the normal DNA of the present invention, it is possible to study the hyperergasia of the protein of the present invention to elucidate the mechanisms of diseases with which the protein of the present invention is associated, and explore therapeutic modalities for the diseases.

Furthermore, the mammal to which the foreign normal DNA of the present invention has been transferred develops symptoms due to an increase in the free protein of the present invention and, therefore, can also be used in the screening of therapeutic drugs for diseases with which the protein of the present invention is associated.

On the other hand, the non-human mammal harboring the foreign abnormal DNA of the present invention can be verified by mating to retain the DNA stably and then bred as a line harboring the DNA from generation to generation under the usual breeding conditions. Moreover, it is possible to incorporate the objective DNA in the above-mentioned plasmid for use as a starting material. The DNA construct with the promoter can be prepared by the routine recombinant DNA technique. Transfer of the abnormal DNA of the present invention in the fertilized egg cell stage insures that the transferred DNA will be ubiquitous in all the germ cells and somatic cells of the host mammal. The presence of the abnormal DNA of the present invention in the germ cells of the transgenic animal means that all the offspring of this transgenic animal harbor the abnormal DNA of the present invention in all of their germ cells and somatic cells. The progeny of this animal harbor the abnormal DNA of the present invention in all of their germ cells and somatic cells. By preparing homozygous male and female animals having the introduced DNA in both homologous chromosomes and mating them, it can be insured that all their offsprings harbor the DNA.

The non-human mammal harboring the abnormal DNA of the present invention features a high expression of the abnormal DNA and, therefore, may eventually develop adiaphoria associated with functional inactivation of the protein of the present invention through inhibition of the function of the endogenous normal DNA and, therefore, can be utilized as an animal model of the disease. For example, by using the transgenic animal harboring the abnormal DNA of the present invention, analysis of the mechanism of this functional inactivation adiaphoria due to the protein of the present invention and therapeutic modalities for the disease can be explored.

As a specific potential use, the transgenic animal with a high expression of the abnormal DNA of the present invention can be used as a model for elucidating the functional inhibition of the normal protein by the abnormal protein of the present invention (dominant negative effect) in adiaphoria of functional inactivation type due to the protein of the present invention. Moreover, the transgenic mammal harboring the foreign abnormal DNA of the present invention develops symptoms due to an decrease in the protein of the present invention and, therefore, can be utilized in the screening of therapeutic compounds for adiaphoria due to functional inactivation of the protein of the present invention.

As other potential uses for transgenic animals harboring the two kinds of DNAs described above, the following uses can be suggested. (1) Use as a cell source for tissue culture; (2) Analysis of the relationship of the protein of the present invention to proteins which are specifically expressed or activated by the protein by direct analysis of DNAs or RNAs in the tissues of the transgenic mammal harboring the DNA of the present invention or analysis of the composition of the protein expressed by the DNA; (3) Study of the functions of cells of those tissues which are generally difficult to culture by using the cells from the tissues containing the DNA as cultured by the standard tissue culture technique; (4) Screening of drugs capable of enhancing the cell functions by using the cells described in (3); (5) Isolation and purification of the muteins of the present invention and construction of antibodies to the muteins.

Furthermore, by using the transgenic animal of the present invention, clinical symptoms of diseases associated with the protein of the present invention, inclusive of said adiaphoria associated with functional inactivation of the protein of the present invention, can be investigated. In addition, more detailed pathological findings can be generated in various organs of this model of diseases associated with the protein of the present invention, thus contributing to the development of new therapies and the study and treatment of secondary diseases arising from such diseases.

Moreover, following isolation of various organs from the transgenic animal of the present invention and their mincing and digestion with a proteolytic enzyme such as trypsin, free single cells harboring the transferred gene can be recovered and cultured for establishment of a cell line. Furthermore, characterization of cells producing the protein of the present invention can be made and their relationship to apotosis, differentiation, or proliferation, the mechanism of signal transduction in them, and abnormalities involved can be explored to thereby generate information useful for a further elucidation of the protein of the present invention and its actions.

Moreover, for the development of therapeutic drugs for diseases associated with the protein of the present invention, such as adiaphoria due to functional inactivation of the protein of the present invention by using the transgenic animal of the present invention, an effective and rapid screening technology for such therapeutic drugs can be established by using the test and assay methods described hereinbefore. In addition, by using the above transgenic animal or the foreign DNA expression vector of the present invention, gene therapies for diseases associated with the protein of the present invention can be explored and developed.

(6) Construction of knockout animals

The present invention further provides a non-human mammalian embryonic stem cell wherein the DNA of the present invention is inactivated and a non-human mammal deficient in expression of the DNA of the present invention wherein the DNA is inactivated.

The present invention, therefore, provides: (1) a non-human mammalian embryonic stem cell wherein the DNA of the present invention is inactivated; (2) the non-human mammalian embryonic stem cell according to in (1) wherein the DNA is inactivated by introduction of a reporter gene (e.g. a β-galactosidase gene of the E. coli origin); (3) the non-human mammalian embryonic stem cell according to (1) which is neomycin-resistant; (4) the non-human mammalian embryonic stem cell according to (1) wherein the non-human mammal is a rodent; (5) the non-human mammalian embryonic stem cell according to (4) wherein the rodent is a mouse; (6) a non-human mammal deficient in expression of the DNA of the present invention, wherein the DNA is inactivated; (7) the non-human mammal according to (6) wherein the DNA is inactivated by introduction of a reporter gene (e.g. a β-galactosidase gene of E. coli origin) and the reporter gene can be expressed under the control of the promoter against the DNA of the present invention; (8) the non-human mammal according to (6) wherein the non-human mammal is a rodent; (9) the non-human mammal according to (8) wherein the rodent is a mouse; and (10) a method for screening for a compound or a salt thereof which enhances or inhibits an activity of the promoter against the DNA of the present invention, which comprises administering a test compound to the non-human mammal according to (7) and detecting an expression of the reporter gene.

The term “non-human mammalian embryonic stem cell wherein the DNA of the present invention is inactivated” means the embryonic stem cell (hereinafter referred to briefly as ES cell) of a non-human mammal in which the DNA has been deprived of the capacity to express the protein of the present invention (hereinafter referred to sometimes as the knockout DNA of the present invention) through introduction of an artificial mutation to the DNA of the present invention possessed by the non-human mammal to thereby inhibit expression of the DNA of the present invention or through substantial deprivation of the activity of the protein of the present invention which is encoded by the DNA.

The non-human mammal includes the same animals mentioned hereinbefore.

Examples of the method for introducing an artificial mutation to the DNA of the present invention are a deletion of some or all of the DNA sequence, or an insertion or substitution of a different DNA by the genetic engineering technology. By such a mutation, the codon reading frame can be shifted or the function of the promoter or exon can be disrupted to provide the knockout DNA of the present invention.

The non-human mammalian embryonic stem cell wherein the DNA of the present invention is inactivated (hereinafter referred to as the ES cell wherein the DNA is an inactivated DNA of the DNA of the present invention or the knockout ES cell of the present invention) can be prepared by, for example, a procedure which comprises isolating the DNA of the present invention from an objective non-human mammal, inserting a drug-resistance gene, typically the neomycin-resistance gene or hygromycin-resistance gene, or a reporter gene such as lacz (β-galactosidase gene) or cat (chloramphenicol acetyltransferase gene) in its exon region to disrupt the function of the exon or inserting a DNA sequence for terminating gene transcription (e.g. poly A coupling signal) in the intron region between exons to thereby inhibit synthesis of a complete mRNA, introducing the thus-constructed DNA chain having a DNA sequence adapted to eventually disrupt the gene (hereinafter referred to briefly as the targeting vector) into the chromosomes of the host animal by homologous recombination, subjecting the resulting ES cell to Southern hybridization analysis using the DNA sequence of the DNA of the present invention or in its vicinity as the probe or a PCR procedure using the DNA sequence of the targeting vector and a DNA sequence in the vicinity but not including the DNA of the present invention used in the construction of the targeting vector as primers, and selecting the knockout ES cell of the present invention.

Moreover, the original ES cell used for inactivation of the DNA of the present invention by the homologous recombination technique or the like may be an already established cell line such as those mentioned hereinbefore or a new cell line established de novo by the known method of Evans and Kaufma. Taking murine ES cells as an example, ES cells of the 129 line are generally employed but the immunological background of this line is not clear. Therefore, the cell line established by using BDF1 mice created by the hybridization of C57BL/6 mice and C57BL/6 mice, both yielding few eggs, with DBA/2 mice (BDF1=F1 of C57BL/6 and DBA/2) for preparing pure-line ES cells with an immunologically defined genetic background can be used with advantage. In addition to the advantage of high egg output and sturdiness of the egg, BDF1 mice have the background of C57BL/6 mice so that in the construction of a disease model with ES cells obtained, the genetic background of the model mice can be converted to that of C57BL/6 mice by back-crossing with C57BL/6.

Moreover, in establishing an ES cell line, it is common practice to use blastocytes 3.5 days following fertilization but, aside from them, a large number of early embryos can be prepared with high efficiency by harvesting the embryos at the 8-cell stage and culturing them into blastocytes.

Furthermore, while ES cells from both male and female animals can be employed, generally ES cells of a male animal are more convenient for the construction of reproduction line chimeras. Moreover, for the purpose of reducing the burden of the complicated cultural procedure, it is preferable to carry out sexing as early as possible.

As a typical method for sexing ES cells, there can be mentioned the method in which the gene in the sex determination region on the Y chromosome is amplified and detected by PCR. Whereas the conventional karyotype analysis requires about 106 cells, the above method requires only about one colony equivalent of ES cells (about 50 cells). Therefore, the primary selection of ES cells in an early stage can be made by this sexing method. Since male cells can thus be selected in the early stage, the trouble in the initial stage of culture can be drastically reduced.

Moreover, the secondary selection can be carried out by G-banding for the number of chromosomes. The number of chromosomes in the resulting ES cell is preferably 100% of the normal number but this goal may not be reached due to the physical and other factors involved in the establishment of the line. In such cases, it is preferable to knockout the gene of the ES cell and reclone it in the normal cell (taking a mouse as an example, the cell in which the number of chromosomes is 2n=40).

The embryonic stem cell line thus established is generally very satisfactory in proliferation characteristic but since it is liable to lose its ontogenic ability, it must be subcultured with sufficient care. For example, this cell line should be cultured on suitable feeder cells such as STO fibroblasts in the presence of LIF (1-10000 U/ml) in a carbon dioxide incubator (preferably 5% CO2-95% air or 5% oxygen-5% CO2-90% air) at about 37 C. and, in subculture, it should be treated with trypsin/EDTA solution (generally 0.001-0.5% trypsin/0.1-5 mM EDTA, preferably about 0.1% trypsin/1 mM EDTA) to provide single cells and seed them on freshly prepared feeder cells. While such subculture is generally performed every 1-3 days, it is good practice to observe the cells on each occasion and, whenever morphologically abnormal cells are discovered, discard the culture.

ES cells can be allowed to differentiate into various types of cells, such as head long muscle cells, visceral muscle cells, heart muscle cells, etc. by con-ducting monolayer culture to a high density under suitable conditions or suspension culture until a mass of cells is formed (M. J. Evans & M. H. Kaufman, Nature, 292, 154, 1981; G. R. Martin, Proceedings of National Academy of Science USA, 78, 7634, 1981; T. C. Doetschman et al., Journal of Embryology and Experimental Morphology, 87, 27, 1985), and the cell deficient in expression of the DNA of the present invention as obtained by causing the ES cell of the present invention to differentiate is useful for the cytobiological in vitro study of the polypeptide of the present invention.

The non-human mammal deficient in expression of the DNA of the present invention can be differentiated from the normal animal by assaying the mRNA in the animals by the known method and comparing the amounts of expression indirectly.

The non-human mammal used for this purpose includes the animals mentioned hereinbefore.

Referring to the non-human mammal deficient in expression of the DNA of the present invention, the DNA of the present invention can be knocked out by introducing the targeting vector constructed as above into, for example, a murine embryonic stem cell or a murine egg cell and thereby causing the DNA sequence of the targeting vector harboring the inactivated DNA of the present invention to undergo homologous recombination with, and accordingly replacing, the DNA of the present invention on the murine embryonic stem cell or egg cell chromosomes.

The cell with the DNA of the present invention thus knocked out can be obtained by Southern hybridization analysis using a DNA sequence of the DNA of the present invention or in its vicinity as a probe or by PCR using a DNA sequence of the targeting vector or a murine-derived DNA sequence in a region adjacent to but not including the DNA of the present invention used in the targeting vector as primers. When a non-human mammalian embryonic stem cell is used, a cell line with the DNA of the present invention knocked out by the homologous recombination technique is cloned and injected into the non-human mammalian embryo or blastocyte at a suitable stage of embryogenesis, for example at the 8-cell stage, and the resulting chimera embryo is transplanted in the pseudopregnant uterus of the non-human mammal. The animal thus obtained is a chimera animal constituted by both the cells harboring the normal DNA of the present invention and the cells harboring the artificially mutated DNA of the present invention.

When some of the gametes of this chimera animal harbor the mutated DNA of the present invention, an individual of which the entire tissues are constituted by cells harboring the mutated DNA of the present invention can be screened from the colony of animals obtained by crossing such a chimera animal with a normal animal, for example by coat color discrimination. The individuals thus selected are usually animals deficient in hetero-expression of the protein of the present invention and by mating such individuals deficient in hetero-expression of the protein of the present invention with each other, animals deficient in homo-expression of the protein of the present invention can be acquired.

When an egg cell is used, a transgenic non-human mammal with the targeting vector having been introduced into its chromosomes can be prepared by injecting the DNA solution into the egg cell nucleus by the microinjection technique and selecting animals expressing a mutation of the DNA of the present invention by homologous recombination.

The individuals with the DNA of the present invention knocked out are mated to verify that the animals obtained by mating also have the DNA knocked out and they can be sub-bred under the usual breeding conditions.

Preparation and maintenance of the reproduction line can also be carried out in the routine manner. Thus, by mating male and female animals harboring the inactivated DNA, homozygotes having the inactivated DNA in both homologous chromosomes can be obtained. The homozygotes thus obtained are bred under such conditions that, with regard to the dam, the number of homozygotes is plural per normal individual. By mating male and female heterozygotes, homozygotes and heterozygotes both harboring the inactivated DNA can be sub-bread.

The non-human mammalian embryonic stem cell harboring the inactivated DNA of the present invention is very useful for the construction of non-human mammals deficient in expression of the DNA of the present invention. Moreover, the mouse deficient in expression of the protein of the present invention lacks the various biological activities inducible by the protein of the present invention and can, there-fore, be of use as an animal model of diseases arising from inactivation of the biological activities of the protein of the present invention, thus being of use in the etiological studies of diseases and development of therapeutics.

In non-human mammals deficient in expression of the DNA of the present invention wherein the DNA of the present invention is inactivated by introducing a reporter gene, the reporter gene is under the control of the promoter for the DNA of the present invention and, therefore, the activity of the promoter can be detected by tracing the expression of the substance encoded by the reporter gene.

For instance, when part of the DNA region coding for the protein of the present present invention has been inactivated by Escherichia coli-derived β- galactosidase gene (lacZ), β-galactosidase is expressed in those tissues in which, the protein of the present invention would have been expressed. Therefore, the status of expression of the protein of the present invention in a living animal body can be traced, easily and expediently, for example, by the staining method using a reagent serving as a substrate for β- galactosidase, such as 5-bromo-4-chloro-3-indolyl-β-galactopyranoside (X-gal). More specifically, a tissue section of a mouse defective in the protein of the present invention is fixed with glutaraldehyde or the like, washed with Dulbecco's phosphate-buffered saline (PBS), and reacted with a staining solution containing X-gal at room temperature or around 37 C. for about 30 minutes to 1 hour. The tissue sample is then washed with 1 mM EDTA/PBS solution to terminate the β-galactosidase reaction and observed for color development. Alternatively, the mRNA coding for lacZ may be detected by a conventional method.

The non-human mammals deficient in expression of the DNA of the present invention is very useful for screening the compounds which activate or inactivate the promoter of the polypeptide of the present invention, and can contribute to find the mechanism of the diseases derived from the deficiency of producing the polypeptide of the present invention or to develop the drug for treating such diseases.

In the specification and drawings of the present application, the abbreviations used for bases (nucleotides), amino acids and so forth are those recommended by the IUPAC-IUB Commission on Biochemical Nomenclature or those conventionally used in the art. Examples thereof are given below. Amino acids for which optical isomerism is possible are, unless otherwise specified, in the L form. DNA: Deoxyribonucleic acid cDNA: Complementary deoxyribonucleic acid A: Adenine T: Thymine G: Guanine C: Cytosine RNA: Ribonucleic acid mRNA: Messenger ribonucleic acid DATP: Deoxyadenosine triphosphate dTTP: Deoxythymidine triphosphate dGTP: Deoxyguanosine triphosphate dCTP: Deoxycytidine triphosphate ATP: Adenosine triphosphate EDTA: Ethylenediamine tetraacetic acid SDS: Sodium dodecyl sulfate EIA: Enzyme Immunoassay G, Gly: Glycine (or Glycyl) A, Ala: Alanine (or Alanyl) V, Val: Valine (or Valyl) L, Leu: Leucine (or Leucyl) I, Ile: Isoleucine (or Isoleucyl) S, Ser: Serine (or Seryl) T, Thr: Threonine (or Threonyl) C, Cys: Cysteine (or Cysteinyl) M, Met: Methionine (or Methionyl) E, Glu: Glutamic acid (or Glutamyl) D, Asp: Aspartic acid (or Aspartyl) K, Lys: Lysine (or Lysyl) R, Arg: Arginine (or Arginyl) H, His: Histidine (or Histidyl) F, Phe: Phenylalamine (or Phenylalanyl) Y, Tyr: Tyrossine (or Tyrosyl) W, Trp: Tryptophan (or Tryptophanyl) P, Pro: Proline (or Prolyl) N, Asn: Asparagine (or Asparaginyl) Q, Gln: Glutamine (or Glutaminyl) pGlu: Pyroglutamic acid (or Pyroglutamyl) Me:

Methyl Et:

Ethyl Bu:

Butyl Ph:

Phenyl TC:

Thiazolidinyl-4(R)-carboxamide

In this specification, substitutions, protective groups and reagents commonly used are indicated by the following abbreviations: BHA

: benzhydrylamine PMBHA: p-methylbenzhydrylamine Tos

: p-toluenesulfonyl CHO

: formyl HONB: N-hydroxy-5-norbornene-2,3-dicarboxyimide OcHex : cyclohexyl ester Bzl

: benzyl Bom

: benzyloxymethyl Br-Z: 2-bromobenzyloxycarbonyl Boc

: t-butoxycarbonyl DCM

: dichloromethane HOBt: 1-hydroxybenztriazole DCC

: N,N′-dicyclohexylcarbodiimide TFA

: trifluoro acetate DIEA: diisopropylethylamine Fmoc: N-9-fluorenylmethoxycarbonyl DNP

: dinitrophenyl Bum

: t-butoxymethyl Trt

: trityl

Each SEQ ID NO set forth in the SEQUENCE LISTING of the specification refers to the following sequence:

SEQ ID NO:1 is an entire amino acid sequence of the murine pituitary-derived ligand polypeptide encoded by the cDNA included in pBOV3.

SEQ ID NO:2 is an entire nucleotide sequence of the murine pituitary-derived ligand polypeptide cDNA.

SEQ ID NO:3 is a genomic nucleotide sequence of the murine pituitary-derived ligand polypeptide cDNA.

SEQ ID NO:4 is an entire amino acid sequence of the matured murine pituitary-derived ligand polypeptide encoded by the cDNA included in pBOV3.

SEQ ID NO:5 is an amino acid sequence of the antigen which can be used for preparation of the anti-ligand polypeptide antibody.

SEQ ID NO:6 is an amino acid sequence of the antigen which can be used for preparation of the anti-ligand polypeptide antibody.

SEQ ID NO:7 is an amino acid sequence of the antigen which can be used for preparation of the anti-ligand polypeptide antibody.

SEQ ID NO:8 is an amino acid sequence of the antigen which can be used for preparation of the anti-ligand polypeptide antibody.

SEQ ID NO:9 is an amino acid sequence of the antigen which can be used for preparation of the anti-ligand polypeptide antibody.

SEQ ID NO:10 is a partial amino acid sequence of the human pituitary-derived G protein-coupled receptor protein encoded by the human pituitary-derived G protein-coupled receptor protein cDNA fragment included in p19P2.

SEQ ID NO:11 is a partial amino acid sequence of the human pituitary-derived G protein-coupled receptor protein encoded by the human pituitary-derived G protein-coupled receptor protein cDNA fragment include in p19P2.

SEQ ID NO:12 is an entire amino acid sequence of the human pituitary-derived G protein-coupled receptor protein encoded by the human pituitary-derived G protein-coupled receptor protein cDNA include in phGR3.

SEQ ID NO:13 is a partial amino acid sequence of the mouse pancreatic β-cell line, MIN6-derived G protein-coupled receptor protein encoded by the mouse pancreatic β-cell line, MIN6-derived G protein-coupled receptor protein cDNA fragment having a nucleotide sequence (SEQ ID NO:18), derived based upon the nucleotide sequences of the mouse pancreatic β-cell line, MIN6-derived G protein-coupled receptor protein cDNA fragments each included in pG3-2 and pG1-10.

SEQ ID NO:14 is a partial amino acid sequence of the mouse pancreatic β-cell line, MIN6-derived G protein-coupled receptor protein encoded by p5S38.

SEQ ID NO:15 is a nucleotide sequence of the human pituitary-derived G protein-coupled receptor protein cDNA fragment include in p19P2.

SEQ ID NO:16 is a nucleotide sequence of the human pituitary-derived G protein-coupled receptor protein cDNA fragment include in p19P2.

SEQ ID NO:17 is an entire nucleotide sequence of the human pituitary-derived G protein-coupled receptor protein cDNA include in phGR3.

SEQ ID NO:18 is a nucleotide sequence of the mouse pancreatic β-cell line, MIN6-derived G protein-coupled receptor protein cDNA, derived based upon the nucleotide sequences of the mouse pancreatic β-cell line, MIN6-derived G protein-coupled receptor protein cDNA fragments each included in pG3-2 and pG1-10.

SEQ ID NO:19 is a nucleotide sequence of the mouse pancreatic β-cell line, MIN6-derived G protein-coupled receptor protein cDNA include in p5S38.

SEQ ID NO:20 is a synthetic DNA primer for screening of cDNA coding for the G protein-coupled receptor protein.

SEQ ID NO:21 is a synthetic DNA primer for screening of cDNA coding for the G protein-coupled receptor protein.

SEQ ID NO:22 is a synthetic DNA primer for screening of cDNA coding for the G protein-coupled receptor protein.

SEQ ID NO:23 is a synthetic DNA primer for screening of cDNA coding for the G protein-coupled receptor protein.

SEQ ID NO:24 is a synthetic DNA primer for screening of cDNA coding for the G protein-coupled receptor protein.

SEQ ID NO:25 is a synthetic DNA primer for screening of cDNA coding for the G protein-coupled receptor protein.

SEQ ID NO:26 is an amino acid sequence of the bovine pituitary-derived ligand polypeptide which was obtained by purification and analysis of N-terminal sequence for P-3 fraction. The amino acid sequence corresponds to 23rd to 51st positions of the amino acid sequence of SEQ ID NO:1.

SEQ ID NO:27 is an amino acid sequence of the bovine pituitary-derived ligand polypeptide which was obtained by purification and analysis of N-terminal sequence for P-2 fraction. The amino acid sequence corresponds to 34th to 52nd positions of the amino acid sequence of SEQ ID NO:1.

SEQ ID NO:28 is entire amino acid sequence of the murine pituitary-derived ligand polypeptide encoded by the cDNA included in pBOV3.

SEQ ID NO:29 is a synthetic DNA primer for screening of cDNA coding for the bovine pituitary-derived ligand polypeptide, wherein the primer is represented by P5-1.

SEQ ID NO:30 is a synthetic DNA primer for screening of cDNA coding for the bovine pituitary-derived ligand polypeptide, wherein the primer is represented by P3-1.

SEQ ID NO:31 is a synthetic DNA primer for screening of cDNA coding for the bovine pituitary-derived ligand polypeptide, wherein the primer is represented by P3-2.

SEQ ID NO:32 is a synthetic DNA primer for screening of cDNA coding for the bovine pituitary-derived ligand polypeptide, wherein the primer is represented by PE.

SEQ ID NO:33 is a synthetic DNA primer for screening of cDNA coding for the bovine pituitary-derived ligand polypeptide, wherein the primer is represented by PDN.

SEQ ID NO:34 is a synthetic DNA primer for screening of cDNA coding for the bovine pituitary-derived ligand polypeptide, wherein the primer is represented by FB.

SEQ ID NO:35 is a synthetic DNA primer for screening of cDNA coding for the bovine pituitary-derived ligand polypeptide, wherein the primer is represented by FC.

SEQ ID NO:36 is a synthetic DNA primer for screening of cDNA coding for the bovine pituitary-derived ligand polypeptide, wherein the primer is represented by BOVF.

SEQ ID NO:37 is a synthetic DNA primer for screening of cDNA coding for the bovine pituitary-derived ligand polypeptide, wherein the primer is represented by BOVR.

SEQ ID NO:38 is an entire amino acid sequence of the bovine genome-derived ligand polypeptide.

SEQ ID NO:39 is an amino acid sequence of the bovine pituitary-derived ligand polypeptide. The amino acid sequence corresponds to 23rd to 53rd positions of the amino acid sequence of SEQ ID NO:1.

SEQ ID NO:40 is an amino acid sequence of the bovine pituitary-derived ligand polypeptide. The amino acid sequence corresponds to 23rd to 54th positions of the amino acid sequence of SEQ ID NO:1.

SEQ ID NO:41 is an amino acid sequence of the bovine pituitary-derived ligand polypeptide. The amino acid sequence corresponds to 23rd to 55th positions of the amino acid sequence of SEQ ID NO:1.

SEQ ID NO:42 is an amino acid sequence of the bovine pituitary-derived ligand polypeptide. The amino acid sequence corresponds to 34th to 53rd positions of the amino acid sequence of SEQ ID NO:1.

SEQ ID NO:43 is an amino acid sequence of the bovine pituitary-derived ligand polypeptide. The amino acid sequence corresponds to 34th to 54th positions of the amino acid sequence of SEQ ID NO:1.

SEQ ID NO:44 is an amino acid sequence of the bovine pituitary-derived ligand polypeptide. The amino acid sequence corresponds to 34th to 55th positions of the amino acid sequence of SEQ ID NO:1.

SEQ ID NO:45 is a synthetic DNA primer for screening of cDNA coding for the murine-derived ligand polypeptide, wherein the primer is represented by RA.

SEQ ID NO:46 is a synthetic DNA primer for screening of cDNA coding for the murine-derived ligand polypeptide, wherein the primer is represented by RC.

SEQ ID NO:47 is a synthetic DNA primer for screening of cDNA coding for the murine-derived ligand polypeptide, wherein the primer is represented by rF.

SEQ ID NO:48 is a synthetic DNA primer for screening of cDNA coding for the murine-derived ligand polypeptide, wherein the primer is represented by rR.

SEQ ID NO:49 is a synthetic DNA primer for screening of cDNA coding for the human-derived ligand polypeptide, wherein the primer is represented by R1.

SEQ ID NO:50 is a synthetic DNA primer for screening of cDNA coding for the human-derived ligand polypeptide, wherein the primer is represented by R3.

SEQ ID NO:51 is a synthetic DNA primer for screening of cDNA coding for the human-derived ligand polypeptide, wherein the primer is represented by R4.

SEQ ID NO:52 is a synthetic DNA primer for screening of cDNA coding for the human-derived ligand polypeptide, wherein the primer is represented by HA.

SEQ ID NO:53 is a synthetic DNA primer for screening of cDNA coding for the human-derived ligand polypeptide, wherein the primer is represented by HB.

SEQ ID NO:54 is a synthetic DNA primer for screening of cDNA coding for the human-derived ligand polypeptide, wherein the primer is represented by HE.

SEQ ID NO:55 is a synthetic DNA primer for screening of cDNA coding for the human-derived ligand polypeptide, wherein the primer is represented by HF.

SEQ ID NO:56 is a synthetic DNA primer for screening of cDNA coding for the human-derived ligand polypeptide, wherein the primer is represented by 5H.

SEQ ID NO:57 is a synthetic DNA primer for screening of cDNA coding for the human-derived ligand polypeptide, wherein the primer is represented by 3HN.

SEQ ID NO:58 is an entire nucleotide sequence of the bovine pituitary-derived ligand polypeptide cDNA.

SEQ ID NO:59 is an entire nucleotide sequence of the murine-derived ligand polypeptide cDNA.

SEQ ID NO:60 is an entire nucleotide sequence of the human-derived ligand polypeptide cDNA.

SEQ ID NO:61 is a synthetic DNA primer for screening of cDNA coding for the murine-derived ligand polypeptide, wherein the primer is represented by rFBG.

SEQ ID NO:62 is a synthetic DNA primer for screening of cDNA coding for the murine-derived ligand polypeptide, wherein the primer is represented by rRSA.

The transformant Escherichia coli, designated INVαF′/p19P2, which is obtained in the Example 2 mentioned herein below, is on deposit under the terms of the Budapest Treaty from Aug. 9, 1994, with the National Institute of Bioscience and Human-Technology (NIBH), Agency of Industrial Science and Technology, Ministry of International Trade and Industry, Japan and has been assigned the Accession Number FERM BP-4776. It is also on deposit from Aug. 22, 1994 with the Institute for Fermentation, Osaka, Japan (IFO) and has been assigned the Accession Number IFO 15739.

The transformant Escherichia coli, designated INVαF′/pG3-2, which is obtained in the Example 4 mentioned herein below, is on deposit under the terms of the Budapest Treaty from Aug. 9, 1994, with NIBH and has been assigned the Accession Number FERM BP-4775. It is also on deposit from Aug. 22, 1994 with IFO and has been assigned the Accession Number IFO 15740.

The transformant Escherichia coli, designated JM109/phGR3, which is obtained in the Example 5 mentioned herein below, is on deposit under the terms of the Budapest Treaty from Sep. 27, 1994, with NIBH and has been assigned the Accession Number FERM BP-4807. It is also on deposit from Sep. 22, 1994 with IFO and has been assigned the Accession Number IFO 15748.

The transformant Escherichia coli, designated JM109/p5S38, which is obtained in the Example 8 mentioned herein below, is on deposit under the terms of the Budapest Treaty from Oct. 27, 1994, with NIBH and has been assigned the Accession Number FERM BP-4856. It is also on deposit from Oct. 25, 1994 with IFO and has been assigned the Accession Number IFO 15754.

The transformant Escherichia coli, designated JM109/pBOV3, which is obtained in the Example 20 mentioned herein below, is on deposit under the terms of the Budapest Treaty from Feb. 13, 1996, with NIBH and has been assigned the Accession Number FERM BP-5391. It is also on deposit from Jan. 25, 1996 with IFO and has been assigned the Accession Number IFO 15910.

The transformant Escherichia coli, designated JM109/pRAV3, which is obtained in the Example 29 mentioned herein below, is on deposit under the terms of the Budapest Treaty from Sep. 12, 1996, with NIBH and has been assigned the Accession Number FERM BP-5665. It is also on deposit from Sep. 3, 1996 with IFO and has been assigned the Accession Number IFO 16012.

The transformant Escherichia coli, designated JM109/pHOV7, which is obtained in the Example 32 mentioned herein below, is on deposit under the terms of the Budapest Treaty from Sep. 12, 1996, with NIBH and has been assigned the Accession Number FERM BP-5666. It is also on deposit from Sep. 5, 1996 with IFO and has been assigned the Accession Number IFO 16013.

The transformant Escherichia coli, designated JM109/pmGB3, which is obtained in the Example 33 mentioned herein below, is on deposit under the terms of the Budapest Treaty from Mar. 3, 1997, with NIBH and has been assigned the Accession Number FERM BP-5666. It is also on deposit from Feb. 19, 1997 with IFO and has been assigned the Accession Number IFO 16059.

The bioactive substance of the present invention, namely the ligand polypeptide or its amide or ester thereof, or a salt thereof, a partial peptide thereof, or the DNA coding for said ligand polypeptide, has function modulating activity for various tissues or internal organs, e.g. heart, lung, liver, spleen, thymus, kidney, adrenal glands, skeltal muscle, testis etc., besides pituitary, central nervous system or pancreas, and are useful as medicines. The substance also is useful for the screening of agonists or antagonists of G protein-coupled receptor proteins. The compounds which can be obtained by such screening also have function modulating activity for above-described tissues or internal organs, and are useful as medicines. Furthermore, the substance is useful for producing a non-human transgenic animal or a non-human knockout animal for analyzing the mechanism of the gene.

### EXAMPLES

Described below are Reference Example and Examples of the present invention which are provided only for illustrative purposes, and not to limit the scope of the present invention.

### Reference Example 1

### Preparation of Synthetic DNA Primers for Amplifying DNA Coding for G protein-coupled receptor Protein

A comparison of deoxyribonucleotide sequences coding for the known amino acid sequences corresponding to or near the first membrane-spanning domain each of human-derived TRH receptor protein (HTRHR), human-derived RANTES receptor protein (L10918, HUMRANTES), human Burkitt's lymphoma-derived unknown ligand receptor protein (X68149, HSBLRlA), human-derived somatostatin receptor protein (L14856, HUMSOMAT), rat-derived μ-opioid receptor protein (U02083, RNU02083), rat-derived β-opioid receptor protein (U00442, U00442), human-derived neuromedin B receptor protein (M73482, HUMNMBR), human-derived muscarinic acetylcholine receptor protein (X15266, HSHM4), rat-derived adrenaline α1B receptor protein (L08609, RATAADRE01), human-derived somatostatin 3 receptor protein (M96738, HUMSSTR3X), human-derived C5a receptor protein (HUMC5AAR), human-derived unknown ligand receptor protein (HUMRDClA), human-derived unknown ligand receptor protein (M84605, HUMOPIODRE) and rat-derived adrenaline α2B receptor protein (M91466, RATA2BAR) was made. As a result, highly homologous regions or parts were found.

Further, a comparison of deoxynucleotide sequences coding for the known amino acid sequences corresponding to or near the sixth membrane-spanning domain each of mouse-derived unknown ligand receptor protein (M80481, MUSGIR), human-derived bombesin receptor protein (L08893, HUMBOMB3S), human-derived adenosine A2 receptor protein (S46950, S46950), mouse-derived unknown ligand receptor protein (D21061, MUSGPCR), mouse-derived TRH receptor protein (S43387, S43387), rat-derived neuromedin K receptor protein (J05189, RATNEURA), rat-derived adenosine A1 receptor protein (M69045, RATALARA), human-derived neurokinin A receptor protein (M57414, HUMNEKAR), rat-derived adenosine A3 receptor protein (M94152, DATADENREC), human-derived somatostatin 1 receptor protein (M81829, HUMSRIlA), human-derived neurokinin 3 receptor protein (S86390, S86371S4), rat-derived unknown ligand receptor protein (X61496, RNCGPCR), human-derived somatostatin 4 receptor protein (L07061, HUMSSTR4Z) and rat-derived GnRH receptor protein (M31670, RATGNRHA) was made. As a result, highly homologous regions or parts were found.

The aforementioned abbreviations in the parentheses are identifiers (reference numbers) which are indicated when GenBank/EMBL Data Bank is retrieved by using DNASIS Gene/Protein Sequencing Data Base (CD019, Hitachi Software Engineering, Japan) and are usually called “Accession Numbers” or “Entry Names”. HTRHR is, however, the sequence as disclosed in Japanese Patent Publication No. 304797/1993 (EPA 638645).

Specifically, it was planned to incorporate mixed bases relying upon the base regions that were in agreement with cDNAs coding for a large number of receptor proteins in order to enhance base agreement of sequences with as many receptor cDNAs as possible even in other regions. Based upon these sequences, the degenerate synthetic DNA having a nucleotide sequence represented by SEQ ID NO:20 or SEQ ID NO:21 which is complementary to the homologous nucleotide sequence were produced.

### Synthetic DNAs

5′-CGTGG (G or C) C (A or C) T (G or C) (G or C) TGGGCAAC (A, G, C or T) (C or T) CCTG-3′ (SEQ ID NO:20)

5′-GT (A, G, C or T) G (A or T) (A or G) (A or G) GGCA (A, G, C or T) CCAGCAGA (G or T) GGCAAA-3′ (SEQ ID NO:21)

The parentheses indicate the incorporation of a plurality of bases, leading to multiple oligonucleotides in the primer preparation. In other words, nucleotide resides in parentheses of the aforementioned DNAs were incorporated in the presence of a mixture of plural bases at the time of synthesis.

### Example 1

### Amplification of Receptor cDNA by PCR Using Human Pituitary Gland-Derived cDNA

By using human pituitary gland-derived cDNA (QuickClone, CLONTECH Laboratories, Inc.) as a template, PCR amplification using the DNA primers synthesized in Reference Example 1 was carried out. The composition of the reaction solution consisted of the synthetic DNA primers (SEQ: 5′ primer sequence and 3′ primer sequence) each in an amount of 1 μm, 1 ng of the template cDNA, 0.25 mM dNTPs, 1 μl of Taq DNA polymerase and a buffer attached to the enzyme kit, and the total amount of the reaction solution was made to be 100 μl. The cycle for amplification including 95 C. for 1 min., 55 C. for 1 min. and 72 C. for 1 min. was repeated 30 times by using a Thermal Cycler (Perkin-Elmer Co.). Prior to adding Taq DNA polymerase, the remaining reaction solution was mixed and was heated at 95 C. for 5 minutes and at 65 C. for 5 minutes. The amplified products were confirmed relying m upon 1.2% agarose gel electrophoresis and ethidium bromide staining.

### Example 2

### Subcloning of PCR Product into Plasmid Vector and Selection of Novel Receptor Candidate Clone via Decoding Nucleotide Sequence of Inserted cDNA Region

The PCR products were separated by using a 0.8% low-melting temperature agarose gel, the band parts were excised from the gel with a razor blade, and were heat-melted, extracted with phenol and precipitated in ethanol to recover DNAs. According to the protocol attached to a TA Cloning Kit (Invitrogen Co.), the recovered DNAs were subcloned into a plasmid vector, pCRTMII (TM represents registered trademark). The recombinant vectors were introduced into E. coli INVαF′ competent cells (Invitrogen Co.) to produce transformants. Then, transformant clones having a cDNA-inserted fragment were selected in an LB agar culture medium containing ampicillin and X-gal. Only transformant clones exhibiting white color were picked with a sterilized toothpick to obtain transformant Escherichia coli INVαF′/p19P2.

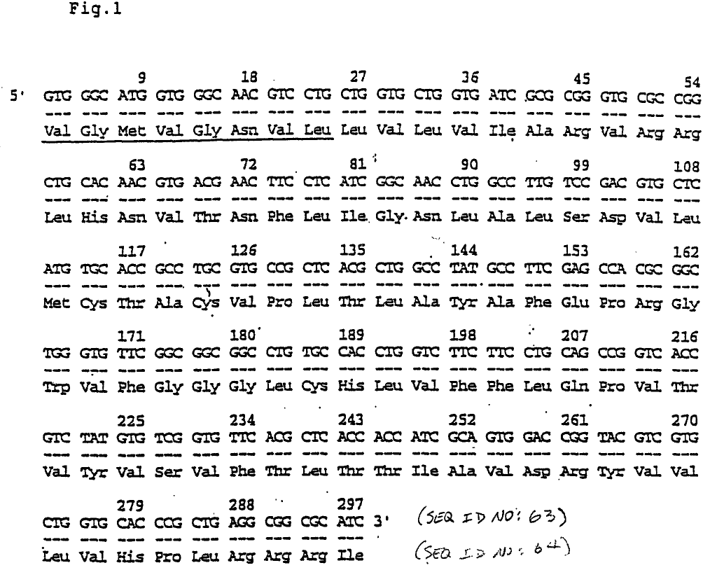

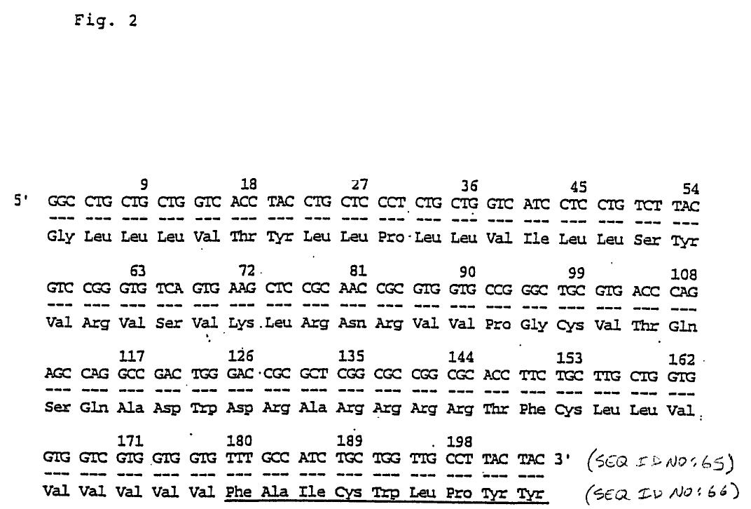

The individual clones were cultured overnight in an LB culture medium containing ampicillin and treated with an automatic plasmid extracting machine (Kurabo Co., Japan) to prepare plasmid DNAs. An aliquot of the DNA thus prepared was cut by EcoRI to confirm the size of the cDNA fragment that was inserted. An aliquot of the remaining DNA was further processed with RNase, extracted with phenol/chloroform, and precipitated in ethanol so as to be condensed. Sequencing was carried out by using a Di-Deoxy terminator cycle sequencing kit (ABI Co.), and the DNAs were decoded by using a fluorescent automatic sequencer, and then the data of the nucleotide sequences obtained were read by using DNASIS (Hitachi System Engineering Co., Japan). The underlined portions of FIG. 1 and FIG. 2 represent regions corresponding to the synthetic primers.

Homology retrieval was carried out based upon the determined nucleotide sequences SEQ ID NO:15 and 16 (Here, the determined nucleotide sequence is the nucleotide sequence which the underlined portion is deleted from the sequence of FIG. 1 or FIG. 2 respectively).

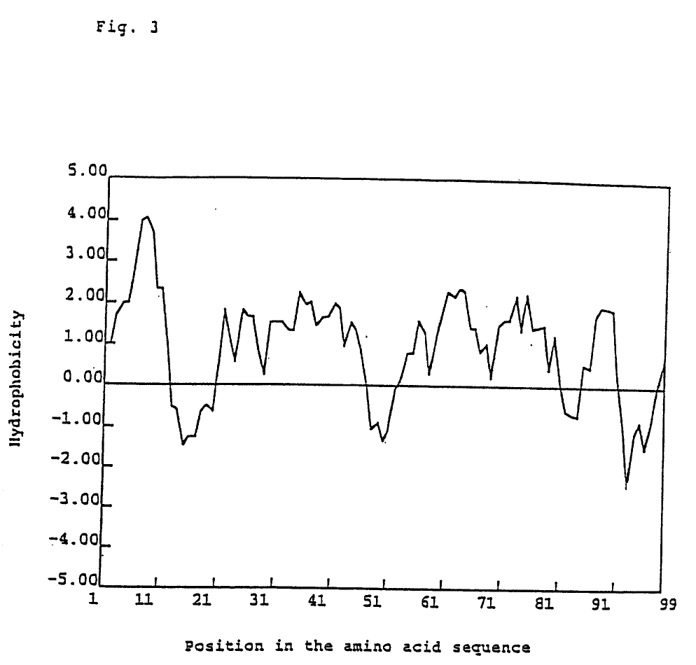

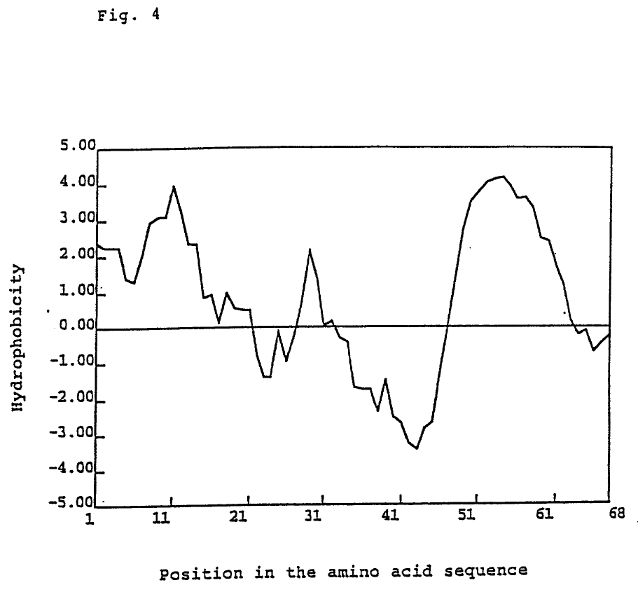

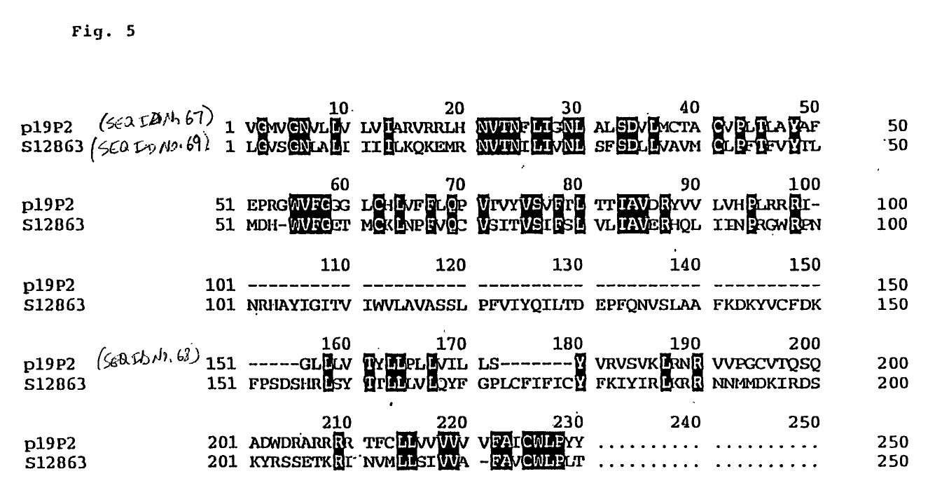

As a result, it was learned that a novel G protein-coupled receptor protein was encoded by the cDNA fragment insert in the plasmid, p19P2, possessed by the transformant Escherichia coli INVαF′/p19P2. To further confirm this fact, by using DNASIS (Hitachi System Engineering Co., Japan) the nucleotide sequences were converted into amino acid sequences [SEQ ID NO:10 and 11], and homology retrieval was carried out in view of hydrophobicity plotting, FIGS. 3 and 4, and at the amino acid sequence level to find homology relative to neuropeptide Y receptor proteins FIG. 5.

### Example 3

### Preparation of Poly(A)+RNA Fraction from Mouse Pancreatic β-Cell Strain, MIN6 and Synthesis of cDNA

A total RNA was prepared from the mouse pancreatic β-cell strain, MIN6 (Jun-ichi Miyazaki et al., Endocrinology, Vol. 127, No. 1, p.126-132) according to the guanidine thiocyanate method (Kaplan B. B. et al., Biochem. J., 183, 181-184 (1979) and, then, poly(A)+RNA fractions were prepared with a mRNA purifying kit (Pharmacia Co.). Next, to 5 μl of the poly(A)+RNA fraction was added a random DNA hexamer (BRL Co.) as a primer, and the resulting mixture was subjected to reaction with mouse Moloney h Leukemia virus (MMLV) reverse transcriptase (BRL Co.) in the buffer attached to the MMLV reverse transcriptase kit to synthesize complementary DNAs. The reaction product was extracted with phenol/chloroform (1:1), precipitated in ethanol, and was then dissolved in 30 μl of TE buffer (10 mM Tris-HCl at pH8.0, 1 mM EDTA at pH8.0).

### Example 4

### Amplification of Receptor cDNA by PCR Using MIN6-Derived cDNA and Sequencing

By using, as a template, 5 μg of cDNA prepared from the mouse pancreatic β-cell strain, MIN6 in the above Example 3, PCR amplification using the DNA primers synthesized in Reference Example 1 was carried out under the same condition as in Example 1. The resulting PCR product was subcloned into the plasmid vector, pCRTMII, in the same manner as in Example 2 to obtain a plasmid, pG3-2. The E. coli INVAF′ was transfected with the plasmid pG3-2 to obtain transformed Escherichia coli INVaF′/pG3-2.

By using, as a template, 5 μg of the CDNA prepared from the mouse pancreatic β-cell strain, MIN6, PCR amplification using DNA primers as disclosed in Libert F. et al., “Science, 244:569- 572, 1989”, i.e., a degenerate synthetic primer represented by the following sequence:

5′-CTGTG (C or T) G (C or T) (G or C) AT (C or T) GCIIT (G or T) GA (C or T) (A or C) G (G or C) TAC-3′ (SEQ ID NO:22)

wherein I is inosine; and a degenerate synthetic primer represented by the following sequence:

5′-A (G or T) G (A or T) AG (A or T) AGGGCAGCCAGCAGAI (G or C) (A or G) (C or T) GAA-3′ (SEQ ID NO:23)

wherein I is inosine, was carried out under the same conditions as in Example 1. The resulting PCR product was subcloned into the plasmid vector, pCRTMII, in the same manner as described in Example 2 to obtain a plasmid, pGl-10.

The reaction for determining the nucleotide sequence (sequencing) was carried out with a DyeDeoxy terminator cycle sequencing kit (ABI Co.), and the DNA was decoded with the fluorescent automatic sequencer (ABI Co.), and then the data of the nucleotide sequence obtained were analyzed with DNASIS (Hitachi System Engineering Co., Japan).

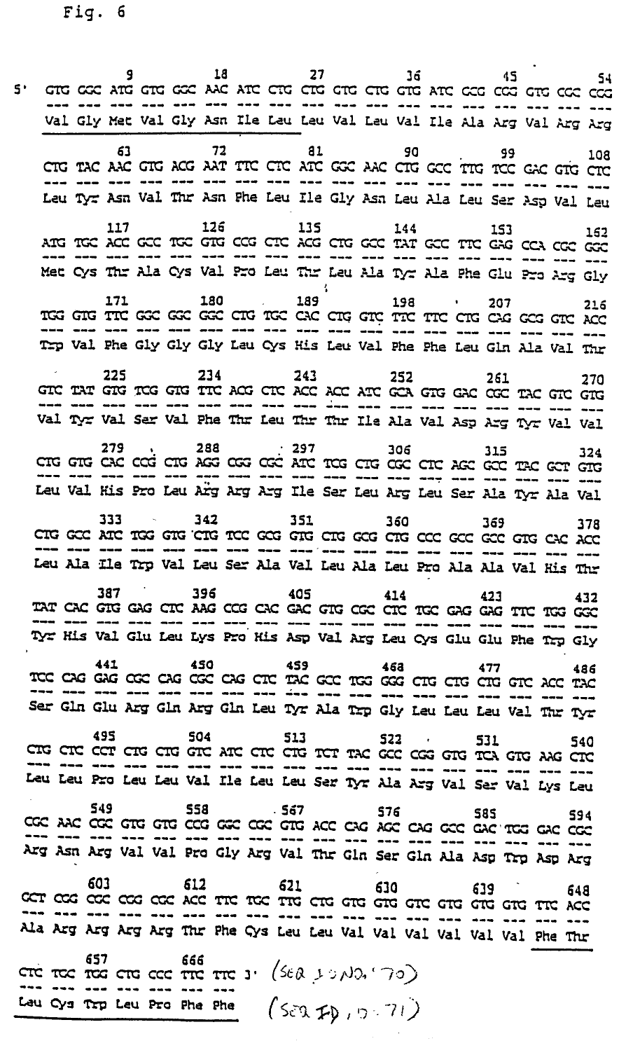

FIG. 6 shows a mouse pancreatic β-cell strain MIN6-derived G protein-coupled receptor protein- encoding DNA (SEQ ID NO:18) and an amino acid sequence (SEQ ID NO:13) encoded by the isolated DNA based upon the nucleotide sequences of plasmids pG3-2 and pGl-10. The underlined portions of FIG. 6 represent regions corresponding to the synthetic primers.

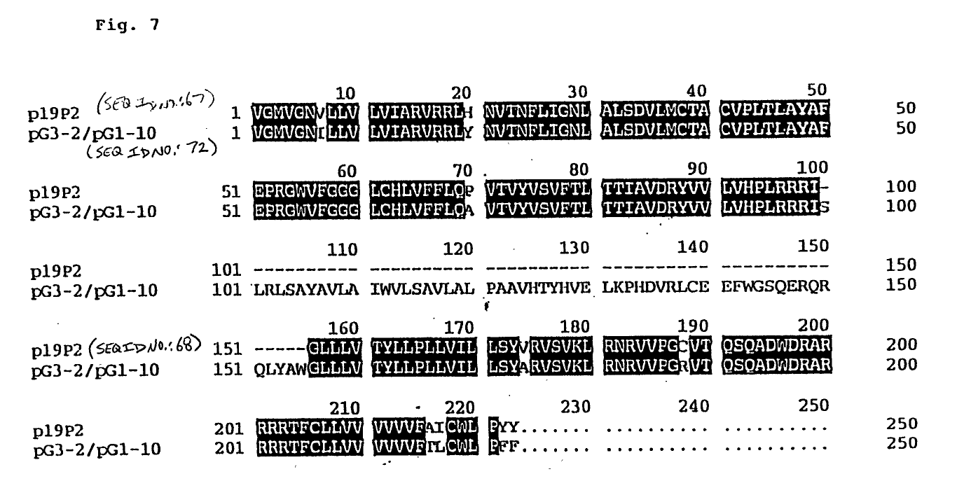

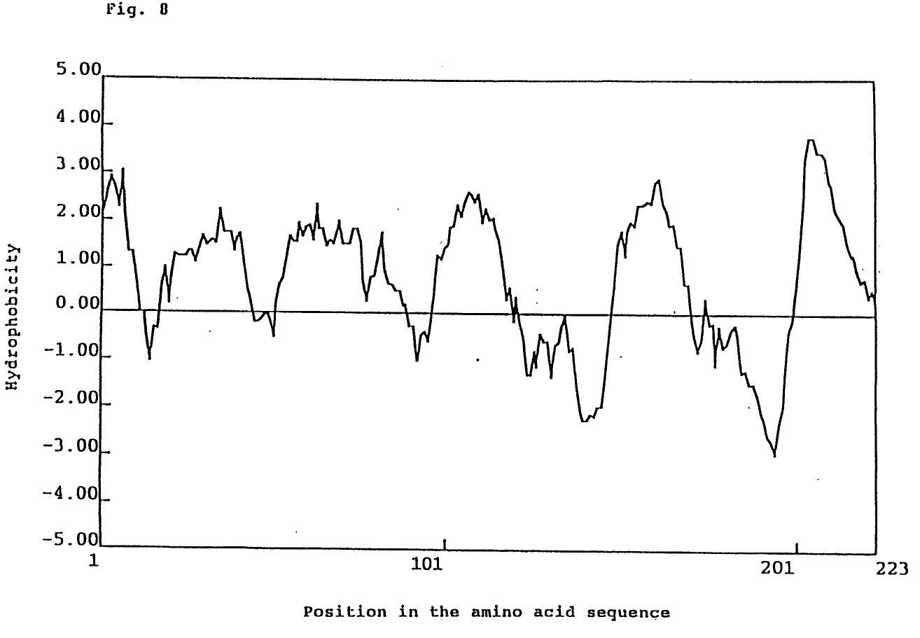

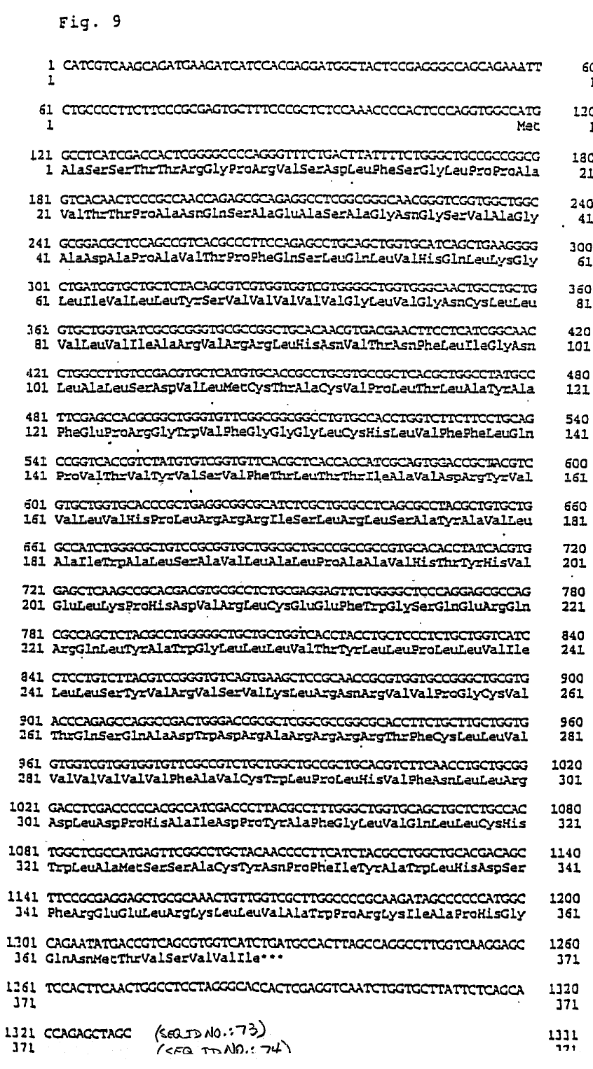

Homology retrieval was carried out based upon the determined nucleotide sequence, FIG. 6. As a result, it was learned that a novel G protein-coupled receptor protein was encoded by the cDNA fragment obtained. To further confirm this fact, by using DNASIS (Hitachi System Engineering Co., Japan) the nucleotide sequence was converted into an amino acid sequence [FIG. 6], and hydrophobicity plotting was carried out to confirm the presence of six hydrophobic regions, FIG. 8. Upon comparing the amino acid sequence with that of p19P2 obtained in Example 2, furthermore, a high degree of homology was found as shown in FIG. 7. As a result, it was strongly suggested that 1g the G protein-coupled receptor proteins encoded by pG3-2 and pGl-10 would recognize the same ligand as the G protein-coupled receptor protein encoded by pl9P2 while the animal species from which the receptor proteins encoded by pG3-2 and pGl-10 were derived was different from the species from which the receptor protein encoded by p19P2 was derived.

Example 5

### Cloning of cDNA Comprising Whole Coding Regions for Receptor Protein from Human Pituitary Gland-Derived cDNA Library

The DNA library constructed by Clontech Co. wherein λgt11 phage vector was used (CLONTECH Laboratories, Inc.; CLH L1139b) was employed as a human pituitary gland-derived cDNA library. The human pituitary gland cDNA library (2×10⁶ pfu (plaque forming units)) was mixed with E. coli Y1090- treated with magnesium sulfate, and incubated at 37 C. for 15 minutes followed by addition of 0.5% agarose (Pharmacia Co.) LB. The E. coli was plated onto a 1.5% agar (Wako-Junyaku Co.) LB plate (containing 50 μg/ml of ampicillin). A nitrocellulose filter was placed on the plate on which plaques were formed so that the plaque was transferred onto the filter. The filter was denatured with an alkali and then heated at 80 C. for 3 hours to fix DNAs.

The filter was incubated overnight at 42 C. together with the probe mentioned herein below in a buffer containing 50% formamide, 5×SSPE (20×SSPE (pH 7.4) is 3 M NaCl, 0.2 M NaH2PO4. H₂O, 25 mM EDTA), 5×Denhardt's solution (Nippon Gene, Japan), 0.1% SDS and 100 μg/ml of salmon sperm DNA for hybridization.

The probe used was obtained by cutting the DNA fragment i inserted in the plasmid, pl19P2, obtained in Example 2, with EcoRI, followed by recovery and labelling by incorporation of [³²P]dCTP (Dupont Co.) with a random prime DNA labelling kit (Amasham Co.).

It was washed with 2×SSC (20×SSC is 3 M NaCl, 0.3 M sodium citrate), 0.1% SDS at 55 C. for 1 hour and, then, subjected to an autoradiography at −80 C. to detect hybridized plaques.

In this screening, hybridization signals were recognized in three independent plaques. Each DNA was prepared from the three clones. The DNAs digested with EcoRI were subjected to agarose gel electrophoresis and were analyzed by the southern blotting using the same probe as the one used in the screening. Hybridizing bands were identified at about 0.7 kb, 0.8 kb and 2.0 kb, respectively. Among them, the DNA fragment corresponding to the band at about 2.0 kb (phGR3) was selected. The phGR3-derived EcoRI fragment with a hybridizable size was subcloned to the EcoRI site of the plasmid, pUC18, and E. coli JM109 was transformed with the plasmid to obtain transformant E. coli JM109/phGR3. A restriction enzyme map of the plasmid, phGR3, was prepared relying upon a restriction enzyme map deduced from the nucleotide sequence as shown in Example 2. As a result, it was learned that it carried a full-length receptor protein-encoding DNA which was predicted from the receptor protein-encoding DNA as shown in Example 2.

### Example 6

### Sequencing of Human Pituitary Gland-Derived Receptor Protein cDNA

Among the EcoRI fragments inserted in the plasmid, phGR3, obtained in the above Example 5, the nucleotide sequence from EcoRI to NheI with about 1330 bp that was considered to be a receptor protein-coding region was sequenced. Concretely speaking, by utilizing restriction enzyme sites that existed in the EcoRI-NheI fragments, unnecessary parts were removed or necessary fragments were subcloned in order to prepare template plasmids for analyzing the nucleotide sequence.

The reaction for determining the nucleotide sequence (sequencing) was carried out with a DyeDeoxy terminator cycle sequencing kit (ABI Co.), and the DNA was decoded with the fluorescent automatic sequencer (ABI Co.), and then the data of the nucleotide sequence obtained were analyzed with DNASIS (Hitachi System Engineering Co., Japan).

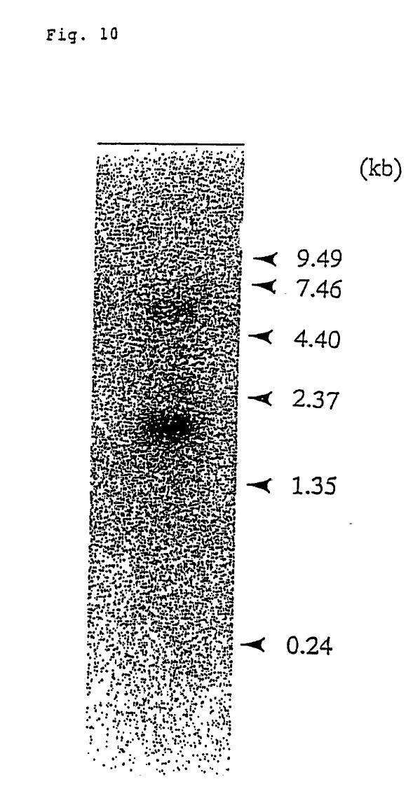

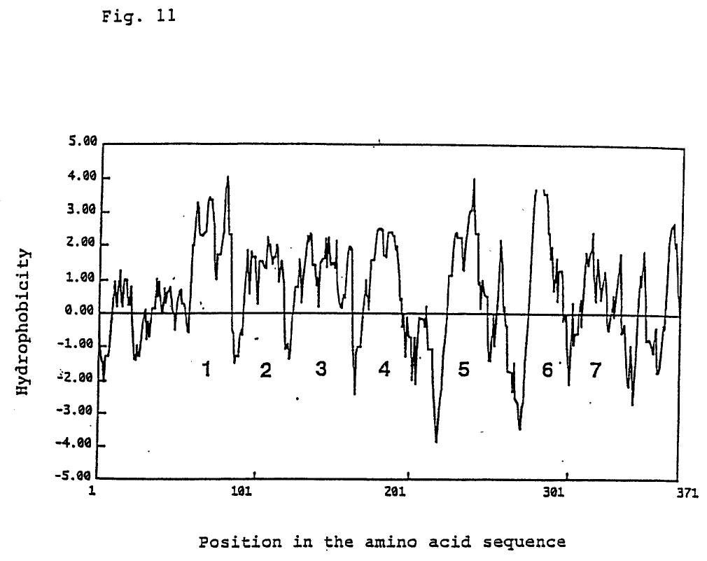

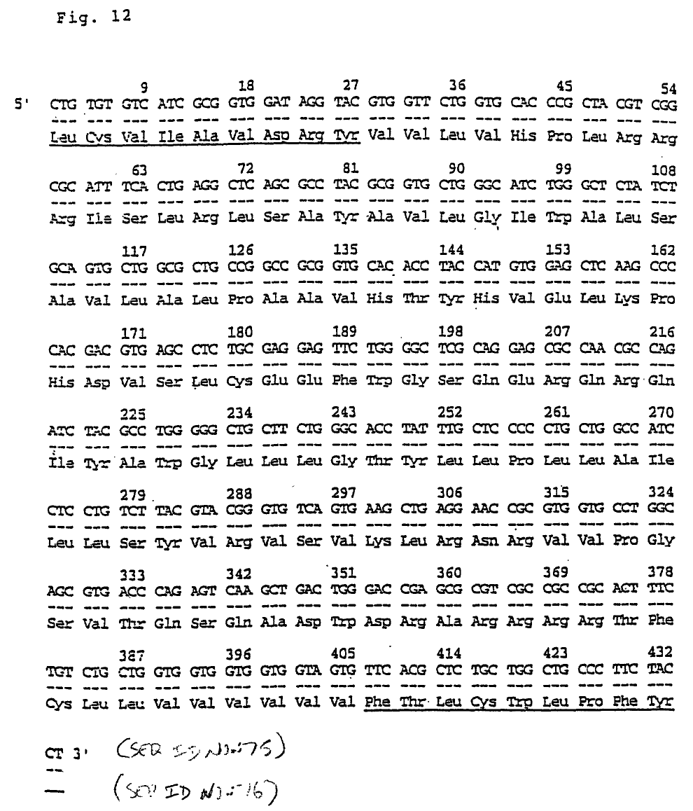

FIG. 9 shows a nucleotide sequence of from just after the EcoRI site up to the NheI site encoded by phGR3. The nucleotide sequence of the human pituitary gland-derived receptor protein-encoding DNA corresponds to the nucleotide sequence (SEQ ID NO:17) of from 118th to 1227th nucleotides, FIG. 9. An amino acid sequence of the receptor protein that is encoded by the nucleotide sequence is shown in SEQ ID NO:12.

### Example 7

### Northern Hybridization with Human Pituitary Gland-Derived Derived Receptor Protein-Encoding phGR3

Northern blotting was carried out in order to detect the expression of phGR3-encoded human pituitary gland-derived receptor proteins obtained in Example 5 in the pituitary gland at a mRNA level. Human pituitary gland mRNA (2.5 pg, Clontech Co.) was used as a template mRNA and the same as the probe used in Example 5 was used as a probe. Nylon membrane (Pall Biodyne, U.S.A.) was used as a filter for northern blotting and migration of the mRNA and adsorption (sucking) thereof with the blotting filter was carried out according to the method as disclosed in Molecular Cloning, Cold Spring Harbor Laboratory Press, 1989.

The hybridization was carried out by incubating the above-mentioned filter and probe in a buffer containing 50% formamide, 5×SSPE, 5×Denhardt's solution, 0.1% SDS and 100 μg/ml of salmon sperm DNA overnight at 42 . The filter was washed with 0.1×SSC, 0.1% SDS at 50 C. and, after drying with air, was exposed to an X-ray film (XAR5, Kodak) for three days at −80 C. The results are as shown in FIG. 10 from which it is considered that the receptor gene encoded by phGR3 is expressed in the human pituitary gland.

### Example 8

### Amplification of Receptor cDNA by PCR Using MIN6-Derived cDNA and Sequencing

By using, as a template, 5 μg of cDNA prepared from the mouse pancreatic β-cell strain, MIN6 in Example 3, PCR amplification using the DNA primers synthesized in Example 4 as disclosed in Libert F. et al., “Science, 244:569-572, 1989”, i.e., a synthetic primer represented by the following sequence:

5′-CTGTG (C or T) G (C or T) (G or C) AT (C or T) GCIIT (G or T) GA (C or T) (A or C) G (G or C) TAC-3′(SEQ ID NO:22)

wherein I is inosine; and a synthetic primer represented by the following sequence:

5′-A (G or T) G (A or T) AG (A or T) AGGGCAGCCAGCAGAI (G or C) (A or G) (C or T) GAA-3′(SEQ ID NO:23)

wherein I is inosine, was carried out under the same conditions as in Example 1. The resulting PCR product was subcloned to the plasmid vector, pCRTMII, in the same manner as in Example 2 to obtain a plasmid, p5S38. E. coli JM109 was transfected with the plasmid p5S38 to obtain transformant Escherichia coli JM109/p5S38.

The reaction for determining the nucleotide sequence (sequencing) was carried out with a DyeDeoxy terminator cycle sequencing kit (ABI Co.), and the DNA was decoded with the fluorescent automatic sequencer (ABI Co.), and then the data of the nucleotide sequence obtained were read with DNASIS (Hitachi System Engineering Co., Japan).

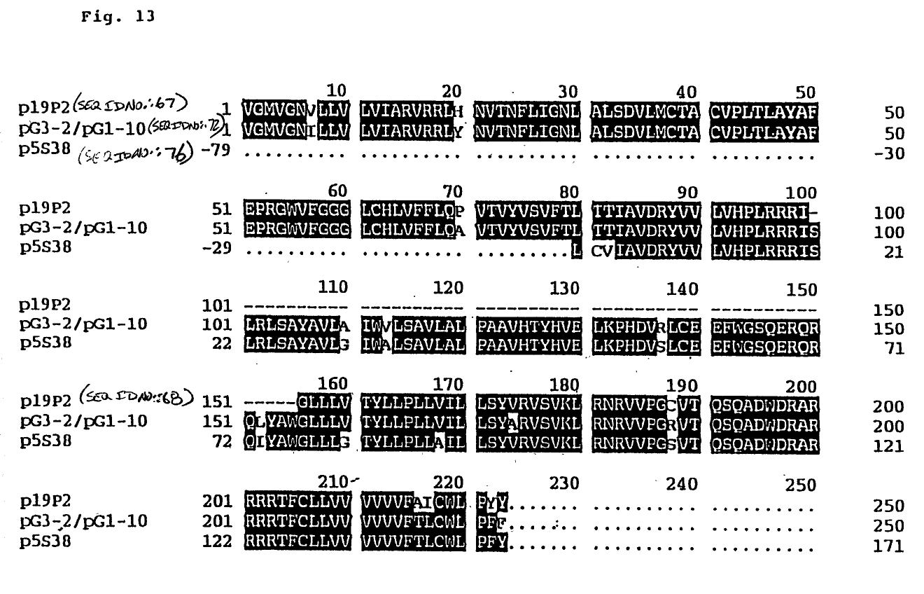

FIG. 12 shows a mouse pancreatic β-cell strain MIN6-derived G protein-coupled receptor protein- encoding DNA (SEQ ID NO:19) and an amino acid sequence (SEQ ID NO:14) encoded by the isolated DNA based upon the nucleotide sequence of plasmid, p5S38. The underlined portions represent regions corresponding to the synthetic primers.

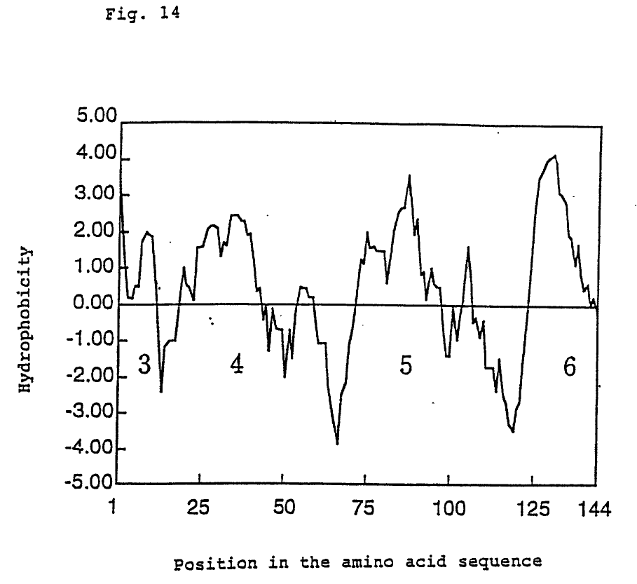

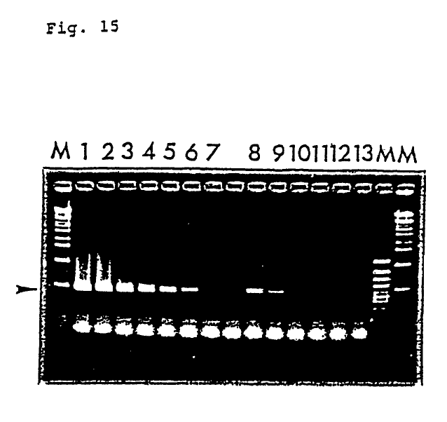

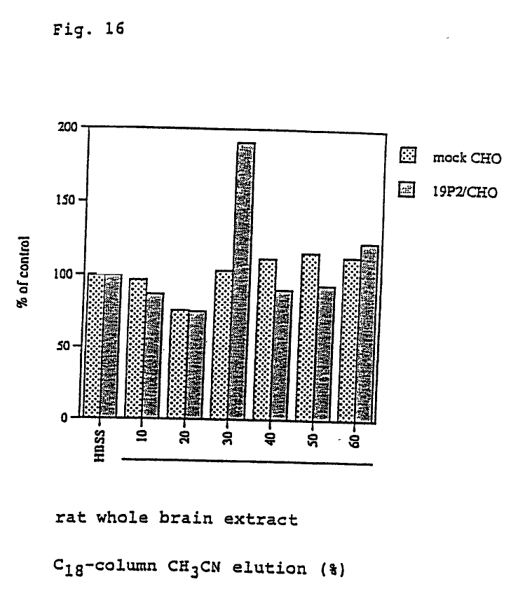

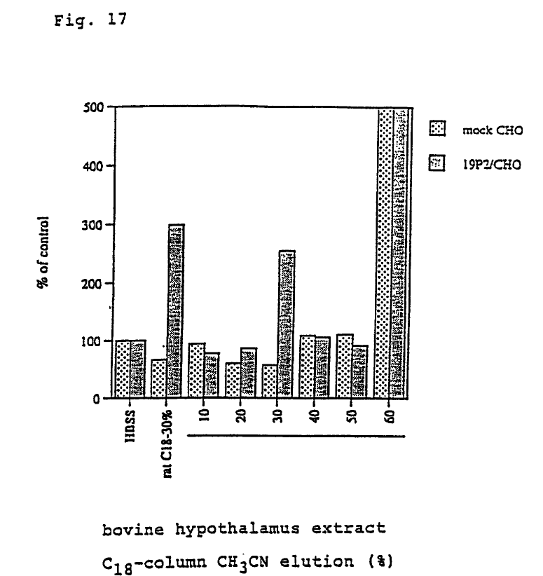

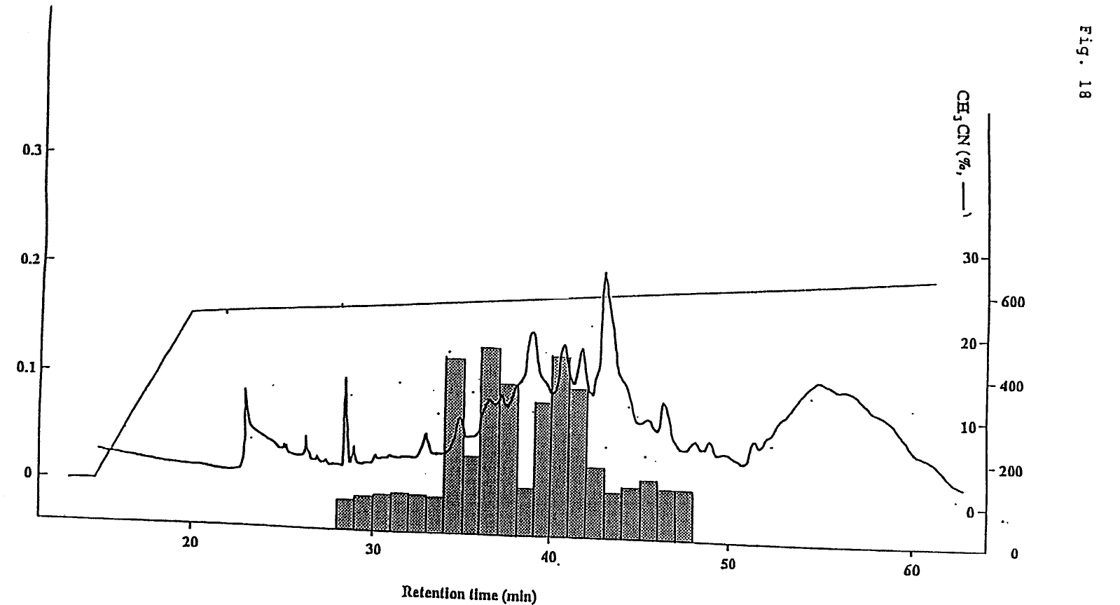

Homology retrieval was carried out based upon the determined nucleotide sequence, FIG. 12. As a result, it was learned that a novel G protein-coupled receptor protein was encoded by the cDNA fragment obtained. To further confirm this fact, by using DNASIS (Hitachi System Engineering Co., Japan), the nucleotide sequence was converted into an amino acid sequence, FIG. 12, and hydrophobicity plotting was carried out to confirm the presence of four hydrophobic regions, FIG. 14. Upon comparing the amino acid sequence with those encoded by p19P2 obtained in Example 2 and encoded by pG3-2 obtained in Example 4, furthermore, a high degree of homology was found as shown in FIG. 13. As a result, it was strongly suggested that the mouse pancreatic β-cell strain, MIN6-derived G protein-coupled receptor protein encoded by p5S38 would recognize the same ligand as the human pituitary gland-derived G protein-coupled receptor protein encoded by p19P2 while the animal species from which the receptor protein encoded by p5S38 was derived was different from the species from which the receptor protein encoded by p19P2 was derived. It was also strongly suggested that the mouse pancreatic β-cell strain, MIN6-derived G protein-coupled receptor protein encoded by p5S38 would recognize the same ligand as the mouse pancreatic β-cell strain, MIN6-derived G protein- coupled receptor proteins encoded by pG3-2 and pG1-10 and they would be analogous receptor proteins one another (so-called “subtype”).

### Example 9

### Preparation of CHO cells which express phGR3

The plasmid phGR3 (Example 5) containing a cDNA encoding the full-length amino acid sequence of human pituitary receptor protein was digested with the restriction enzyme Nco I and electrophoresed on agarose gel and a fragment of about 1 kb was recovered. Both ends of the recovered fragment were blunted with a DNA blunting kit (Takara Shuzo Co., Japan) and, after a SalI linker was added, the fragment was treated with SalI and inserted into the SalI site of pUC119 to provide plasmid S10. Then, S10 was treated with SalI and SacII to prepare a fragment of about 700 bp (containing the N-terminal coding region). Then, a fragment of about 700 bp (containing the C-terminal coding region including initiation and termination codons) was cut out from phGR3 with Sac II and Nhe I. These two fragments were added to the animal cell expression vector plasmid pAKKO-111H (the vector plasmid identical to the pAKKO1.11 H described in Biochim. Biophys. Acta, Hinuma, S., et al., 1219 251- 259, 1994) and a ligation reaction was carried out to construct a full-length receptor protein expression plasmid pAKKO-19P2.

E. coli transfected with pAKKO-19P2 was cultured and the pAKKO-19P2 plasmid DNA was mass-produced using QUIAGEN Maxi. A 20 μg portion of the plasmid DNA was dissolved in 1 ml of sterile PBS, and in a gene transfer vial (Wako Pure Chemical Ind.), the solution was vortexed well for liposome formation. This liposome, 125 μl, was added to CHOdhfr- cells previously subcultured at 1×10⁶ cells per locm-dia. dish 24 hr and placed in fresh medium immediately before addition and overnight culture was carried out. After a further one-day culture in fresh medium, the medium was changed to a screening medium and the incubation was further carried out for a day. For efficient screening of transformants, subculture was carried out at a low cell density and only the cells growing in the screening medium were selected to establish a full-length receptor protein expression CHO cell line CHO-19P2.

### Example 10

### Confirmation of the amount of expression of the full-length receptor protein in the CHO-19P2 cell line at the transcription level

Using FastTrack Kit (Invitrogen), CHO cells transfected with pAKKO-19P2 according to the kit manual and mock CHO cells were used to prepare poly(A)+RNA. Using 0.02 μg of this poly(A)+RNA, a CDNA was synthesized by means of RNA PCR Kit (Takara Shuzo, Co., Japan). The kind of primer used was a random 9mer and the total volume of the reaction mixture was 40 μl. As a negative control of cDNA synthesis, a reverse transcriptase-free reaction mixture was also provided. First, the reaction mixture was incubated at 30 C. for 10 minutes to conduct an amplification reaction to some extent. Then, it was incubated at 42 C. for 30 minutes to let the reverse transcription reaction proceed. The enzyme was inactivated by heating at 99 C. for 5 minutes and the reaction system was cooled at 5 C. for 5 minutes.

After completion of the reverse transcription reaction, a portion of the reaction mixture was recovered and after dilution with distilled water, extraction was carried out with phenol/chloroform and further with diethyl ether. The extract was subjected to precipitation from ethanol and the precipitate was dissolved in a predetermined amount of distilled water for use as a cDNA sample. This cDNA solution and the plasmid DNA (PAKKO-19P2) were serially diluted and using primers specific to full-length receptor protein, PCR was carried out. The sequences of the primers prepared according to the base sequence of the coding region of the full-length receptor protein were CTGACTTATTTTCTGGGCTGCCGC (SEQ ID NO:24) for 5′ end and AACACCGACACATAGACGGTGACC (SEQ ID NO:25) for 31 end.

The PCR reaction was carried out in a total volume of 100 μl using 1 MM each of the primers, 0.5 μl of Taq DNA polymerase (Takara Shuzo Co., Japan), the reaction buffer and dNTPs accompanying the enzyme, and 10 μl of template DNA (cDNA or plasmid solution). First the reaction mixture was heat-treated at 94 C. for 2 minutes for sufficient denaturation of the template DNA and subjected to 25 cycles of 95 C.×30 seconds, 65 C.×30 seconds, and 72 C.×60 seconds. After completion of the reaction, 10 μl of the reaction mixture was subjected to agarose gel electrophoresis and the detection and quantitative comparison of amplification products were carried out. As a result, a PCR product of the size (400 bp) predictable from the sequence of the cDNA coding for the full-length receptor protein was detected, FIG. 15. In the lane of the PCR reaction mixture using the product of the reverse transcriptase- free transcription system as the template, no specific band was detected, thus extruding the possibility of its being a PCR product derived from the genomic DNA of CHO cells. Moreover, no specific band appeared in the lane of mock cells, either. Therefore, it was clear that the product was not derived from the mRNA initially expressed in CHO cells FIG. 15.

### Example 11

### Detection of the activity to specifically promote release of arachidonic acid metabolites from CHO-19P2 cells in a rat whole brain extract

A crude peptide fraction was prepared from rat whole brain by the following procedure. The rat whole brain enucleated immediately after sacrifice was frozen in liquefied nitrogen and stored at −80 C. The frozen rat whole brain, 20 g (the equivalent of 10 rats) was finely divided and boiled in 80 ml of distilled water for 10 minutes. After the boiled tissue was quenched on ice, 4.7 ml of acetic acid was added at a final concentration of 1.0 M and the mixture was homogenized using a Polytron (20,000 rpm, 6 min.). The homogenate was stirred overnight and then centrifuged (10,000 rpm, 20 min.) to separate the supernatant. The sediment was homogenized in 40 ml of 1.0 M acetic acid and centrifuged again to recover the supernatant. The supernatants were pooled, diluted in 3 volumes of acetone, allowed to stand on ice for 30 minutes, and centrifuged (10,000 rpm, 20 min.) to recover the supernatant. The recovered supernatant was evaporated to remove acetone. To the resulting acetone-free concentrate was added 2 volumes of 0.05% trifluoroacetic acid(TFA)/ H₂O and the mixture was applied to a reversed-phase C18 column (Prep C18 125 Å, Millipore). After application of the supernatant, the column was washed with 0.05% TFA/ H₂O, and gradient elution was carried out with 10%, 20%, 30%, 40%, 50%, and 60% CH₃CN/0.05%TFA/ H₂O. The fractions were respectively divided into 10 equal parts and lyophilized. The dried sample derived from one animal equivalent of rat whole brain was dissolved in 20 μl of dimethyl sulfoxide (DMSO) and suspended in 1 ml of Hank's balanced saline solution (HBSS) supplemented with 0.05% bovine serum albumin (BSA) to provide a crude peptide fraction.

The full-length receptor protein-expressed CHO cells and mock CHO cells were seeded in a 24-well plate, 0.5×10⁵ cells/well, and cultured for 24 hours. Then, [³H] arachidonic acid was added at a final concentration of 0.25μCi/well. Sixteen (16) hours after addition of [³H] arachidonic acid, the cells were rinsed with 0.05% BSA-HBSS and the above-mentioned crude peptide fraction was added, 400 μl/well. The mixture was incubated at 37 C. for 30 minutes and a 300 μl portion of the reaction mixture (400 μl) was added to a scintillator (4 ml) and the amount of [³H] arachidonic acid metabolite released into the reaction mixture was determined with a scintillation counter. As a result, an arachidonic acid metabolite-releasing activity specific to the full-length receptor protein expressed CHO cells (CHO-19P2) was detected in the 30% CH₃CN fraction of the eluate FIG. 16.

### Example 12

### Detection of the activity to specifically promote release of arachidonic acid metabolites from CHO-19P2 cells in a bovine hypothalamus extract

A crude peptide fraction was prepared from 360 g (the equivalent of 1 animals) of bovine brain tissue including hypothalamus in the same manner as in Example 11. A dried peptide sample per 0.05 animal was dissolved in 40 μl of DMSO and suspended in 2 ml of 0.05% BSA-HBSS and the detection of A arachidonic acid metabolite-releasing activity was attempted in the same manner as in Example 11. As a result, the activity to specifically promote release of arachidonic acid metabolites from the CHO-19P2 cell line was detected in the fraction eluted with 30% CH₃CN from a C18 column to which the crude bovine hypothalamus peptide fraction had been applied, FIG. 17.

### Example 13

### Preparation of the activity (peptide) to specifically promote release of arachidonic acid metabolites from CHO-19P2 cells by purification from bovine hypothalamus

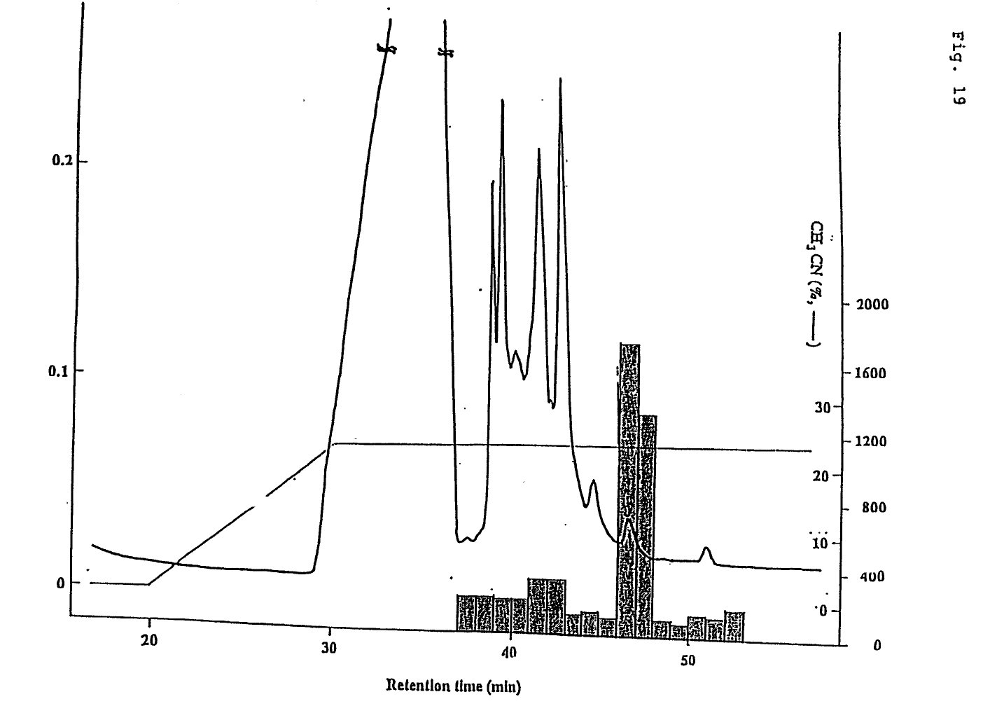

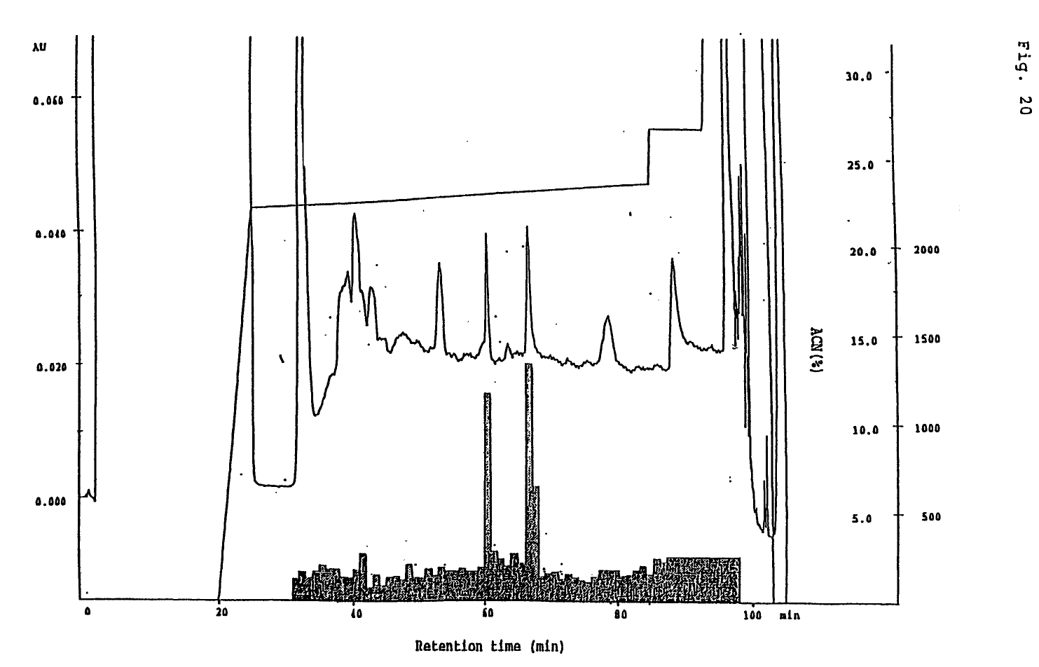

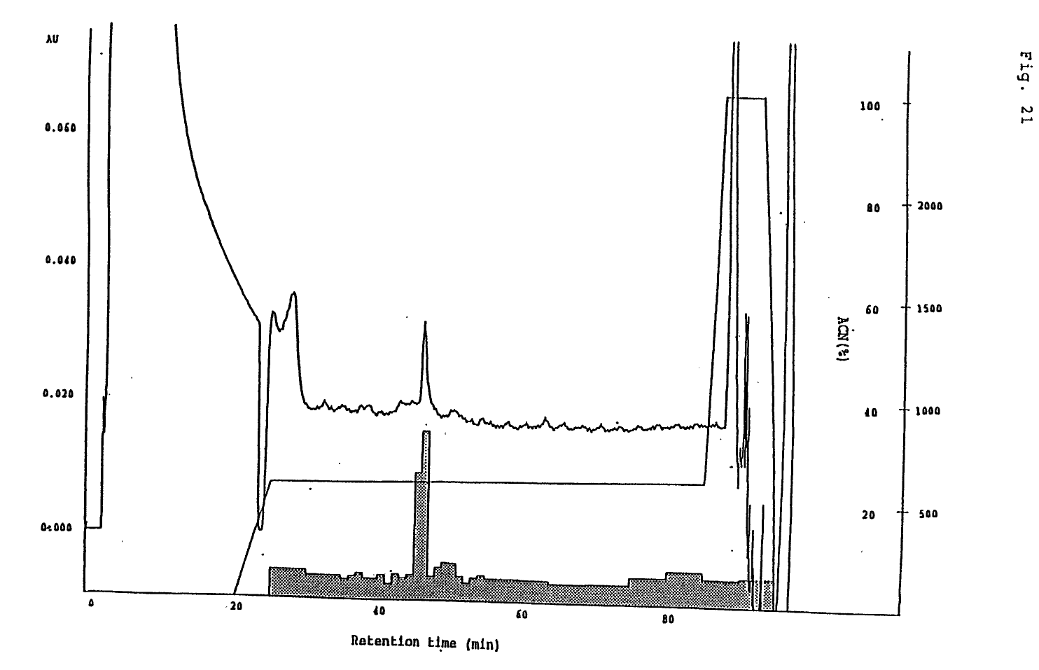

A typical process for harvesting the activity to specifically promote release of arachidonic acid metabolites from the CHO-19P2 cell line by purification from bovine hypothalamus is now described. A frozen bovine brain tissue specimen including hypothalamus, 4.0 kg (the equivalent of 80 animals) was ground and boiled in 8.0 L of distilled water for 20 minutes. After quenching on ice, 540 ml of acetic acid was added at a final concentration of 1.0 M and the mixture was homogenized using a Polytron (10,000 rpm, 12 min.). The homogenate was stirred overnight and then centrifuged (9,500 rpm, 20 min) to recover a supernatant. The sediment was suspended in 4.0 L of 1.0 M acetic acid and homogenized with the Polytron and centrifuged again to recover a further supernatant. The supernatants were pooled and TFA was added at a final concentration of 0.05%. The mixture was applied to reversed-phase C18 (Prep C18 125 Å, 160 ml; Millipore) packed in a glass column. After addition, the column was washed with 320 ml of 0.05% TFA/H₂O and 3-gradient elution was carried out with 10%, 30%, and 50% CH₃CN/0.05% TFA/H₂O. To the 30% CH₃CN/0.05% TFA/H₂O fraction was added 2 volumes of 20 mM CH₃COONH₄/H₂O and the mixture was applied to the cation exchange column HiPrep CM-Sepharose FF (Pharmacia). After the column was washed with 20 mM CH₃COONH₄/10% CH3CN/H₂O, 4- gradient elution was carried out with 100 mM, 200 mM, 500 mM, and 1000 mM CH₃COONH₄/10% CH₃CN/H₂O. In the 200 mM CH3COONH4 fraction, activity to specifically promote release of arachidonic acid metabolites from CHO-19P2 was detected. Therefore, this fraction was diluted with 3 volumes of acetone, centrifuged for deproteination, and concentrated in an evaporator. To the concentrated fraction was added TFA (final concentration 0.1%) and the mixture was adjusted to pH 4 with acetic acid and applied to 3 ml of the reversed- phase column RESOURCE RPC (Pharmacia). Elution was carried out on a concentration gradient of 15%-30% CH₃CN. As a result, activity to specifically promote the release of arachidonic acid metabolites from the CHO-19P2 cell line was detected in the 19%-21% CH₃CN fraction. The active fraction eluted from RESOURCE RPC was lyophilized, dissolved with DMSO, suspended in 50 mM MES pH 5.0/10% CH3CN, and added to 1 ml of the cation exchange column RESOURCE S. Elution was carried out on a concentration gradient of 0 M-0.7 M NaCl. As a result, the activity to specifically promote release of arachidonic acid metabolites from CHO-19P2 cells was detected in the 0.32 M-0.46 M NaCl fraction. The active eluate from RESOURCE S was lyophilized, dissolved with DMSO, suspended in 0.1% TFA/H₂O, and added to reversed-phase column C18 218TP5415 (Vydac), and elution was carried out on a concentration gradient of 20%-30% CH₃CN. As a result, the activity to specifically promote release of arachidonic acid metabolites from CHO-19P2 cells was detected in the three fractions 22.5%, 23%, and 23.5% CH₃CN (these active fractions are designated as P-1, P-2, and P-3) [FIG. 18]. Of the three active fractions, the 23.5% CH₃CN fraction (P-3) was lyophilized, dissolved with DMSO, suspended in 0.1% TFA/ H₂0, and added to the reversed-phase column diphenyl 219TP5415 (Vydac), and elution was carried out on a gradient of 22%-25% CH₃CN. As a result, the activity to specifically promote release of arachidonic acid metabolites from CHO-19P2 cells was converged by recovery in one elution peak obtained with 23% CH₃CN [FIG. 19]. The active peak fraction from the reverse-phased column diphenyl 219TP5415 was lyophilized, dissolved with DMSO, suspended in 0.1% TFA/ H₂O, and added to the reversed- phase column μRPC C2/C18 SC 2.1/10 (Pharmacia), and elution was carried out on a gradient of 22%-23.5% CH₃CN. As a result, the activity to specifically promote release of arachidonic acid metabolites from CHO-19P2 cells was detected in the two peaks eluted with 23.0% and 23.2% CH₃CN, FIG. 20.

### Example 14

### Determination of the amino acid sequence of the peptide having the activity to specifically promote release of arachidonic acid metabolites from CHO-19P2 cells as purified from bovine hypothalamus

The amino acid sequence of the peptide (P-3) having activity to specifically promote release of arachidonic acid metabolites from CHO-19P2 cells as purified in Example 13 was determined. The fraction showing peak activity from the reversed-phase pRPC C2/C18 SC 2.1/10 was lyophilized and dissolved in 20 μl of 70% CH₃CN and analyzed for amino acid sequence with the peptide sequencer (ABI.491). As a result, the sequence defined by SEQ ID NO:26 was obtained. However, the 7th and 19th amino acids were not determined by only the analysis of amino acid sequence.

### Example 15

### Preparation of the active substance (peptide) which specifically promotes release of arachidonic acid metabolites from CHO-19P2 cells as purified from bovine hypothalamus

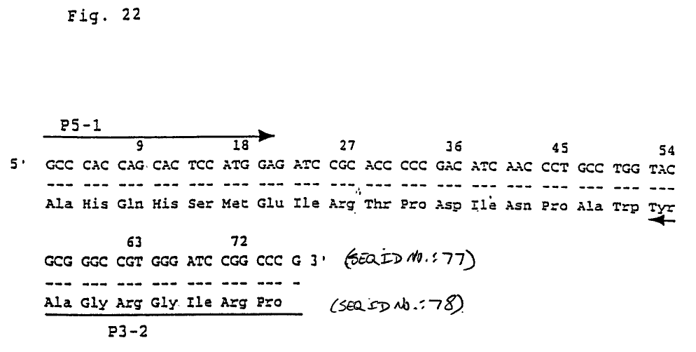

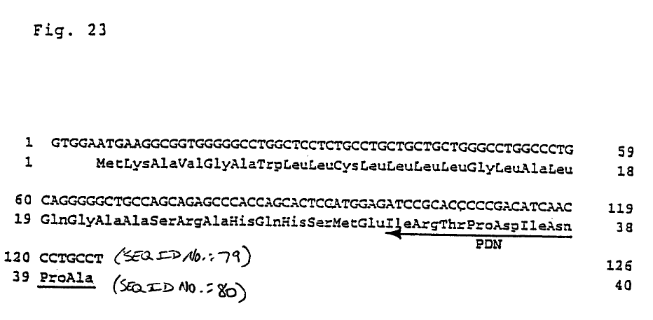

Of the three active fractions obtained with Vydac C18 218TP5415 in Example 13, the active fraction (P-2) eluted with 23.0% CH₃CN was further purified. This active fraction was lyophilized, dissolved with DMSO, suspended in 0.1% TFA/dH₂O, and added to reversed-phase column diphenyl 219TP5415 (Vydac), and elution was carried out on a gradient of 21.0%-24.0% CH₃CN. As a result, activity to specifically promote release of arachidonic acid metabolites from CHO-19P2 cells was detected in a peak eluted with 21.9% CH₃CN. This fraction was lyophilized, dissolved with DMSO, suspended in 0.1% TFA/d H₂0, and added to reversed-phase μLRPC C2/C18 SC 2.1/10 (Pharmacia), and then elution was carried out on a CH₃CN gradient of 21.5%-23.0%. As a result, the activity to specifically promote release of arachidonic acid metabolites from CHO-19P2 cells converged in one peak eluted with 22.0% CH₃CN, FIG. 21.

### Example 16

### Determination of the amino acid sequence of the peptide (P-2) purified from bovine hypothalamus which specifically promotes release of arachidonic acid metabolites from CHO-19P2 cells

The amino acid sequence of the peptide (P-2) having the activity to specifically promote release of arachidonic acid metabolites from CHO-19P2 cells as purified in Example 15 was determined. The active peak fraction from the reversed-phase column μRPC C2/Cl8 SC 2.1/10 was lyophilized, dissolved in 20 μl of 70% CH₃CN, and analyzed for amino acid sequence with the peptide sequencer (ABI, 492) (SEQ ID NO:27).

### Example 17

### Preparation of a poly(A)+RNA fraction from bovine hypothalamus and synthesis of a cDNA

Using Isogen (Nippon Gene), total RNA was prepared from one animal equivalent of bovine hypothalamus. Then, using Fast Track (Invitrogen), a poly(A)+RNA fraction was prepared. From 1 μg of this poly(A)+RNA fraction, cDNA was synthesized using 3′ RACE system (GIBCO BRL) and Marathon cDNA amplification kit (Clontech) according to the manuals and dissolved in 20 and 10 μl, respectively.

### Example 18

### Acquisition of cDNA coding for the amino acid sequence established in Example 14

To obtain a cDNA coding for a polypeptide comprising the amino acid sequence established in Example 14, the acquisition of a base sequence coding for SEQ ID NO:28 was attempted in the first place. Thus, primers P5-1 (SEQ ID NO:29), P3-1 (SEQ ID NO:30), and P3-2 (SEQ ID NO:31) were synthesized. (In the Sequence Table, I represents inosine). Using 0.5 μl of the cDNA prepared by 3′ RACE in Example 17 as a template and EXTaq (Takara Shuzo Co., Japan) as DNA polymerase, 2.5 μl of accompanying buffer, 200 μM of accompanying dNTP, and primers P5-1 and P3-1 were added each at a final concentration of 200 nM, with water added to make 25 μl, and after treatment for one minute at 94, the cycle of 98 C.×10 seconds, 50 C.×30 seconds, 68 C.×10 seconds was repeated 30 times. This reaction mixture was diluted 50-fold with tricin-EDTA buffer and using 2.5 μl of the dilution as a template and the primer combination of P5-1 and P3-2, the reaction was carried out in otherwise the same manner as described above. As the thermal cycler, Gene Amp 9600 (Perkin Elmer) was used. The amplification product was subjected to 4% agarose gel electrophoresis and ethidium bromide staining and a band of about 70 bp was cut out and subjected to thermal fusion, phenol extraction, and ethanol precipitation. The recovered DNA was subcloned into plasmid vector PCRTMII according to the manual of TA Cloning kit (Invitrogen). The vector was then introduced into E. coli JM109 and the resultant transformant was cultured in ampicillin-containing LB medium. The plasmid obtained with an automatic plasmid extractor (Kurabo) was reacted according to the manual of DyeDeoxy Terminator Cycle Sequencing Kit (ABI) and decoded with a fluorescent automatic DNA sequencer (ABI). As a result, the sequence shown in FIG. 22 was obtained and confirmed to be part of the base sequence coding for SEQ ID NO:28.

### Example 19

### Acquisition of a bioactive polypeptide cDNA by RACE using the sequence established in Example 18

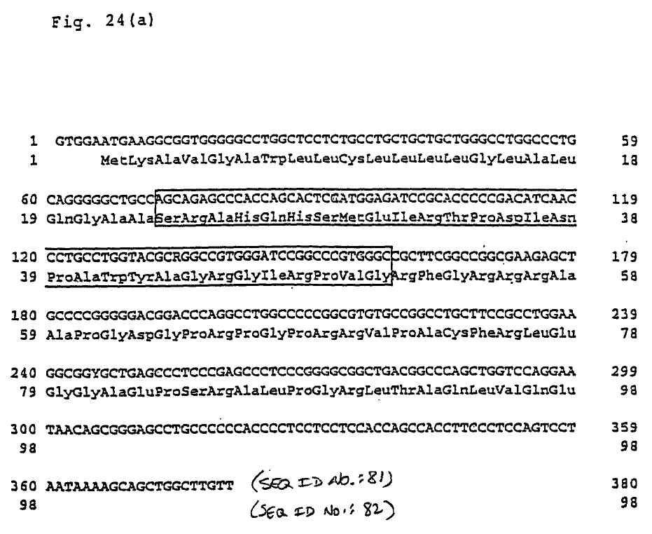

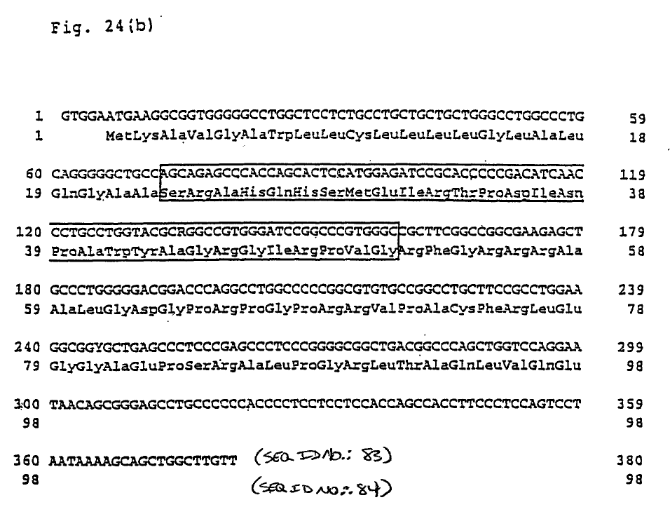

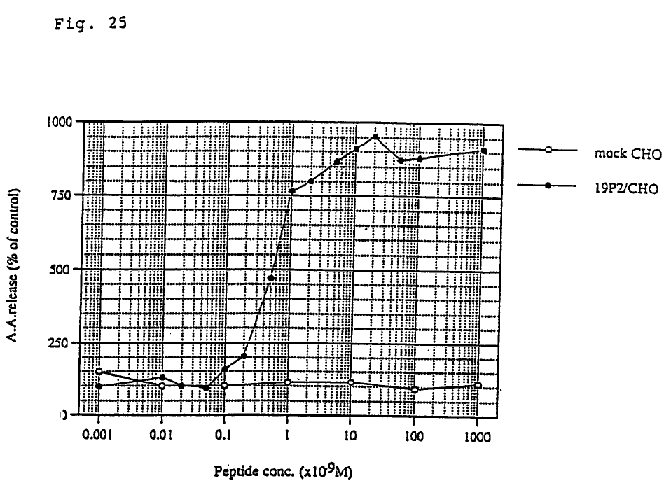

First, for amplification (5′ RACE) of the sequence at 5′ end, the two primers PE (SEQ ID NO:32) and PDN (SEQ ID NO:33) were synthesized by utilizing the sequence shown in FIG. 22. The cDNA prepared using Marathon cDNA amplification kit in Example 17 was diluted 100-fold with tricin-EDTA buffer. Then, in the same manner as Example 2, a reaction mixture was prepared using 2.5 μl of the dilution and a combination of the adapter primer AP1 accompanying the kit and the primer PE and after treatmenet for one minute at 94 C., the cycle of 98 C.×10 seconds and 68 C.×5 minutes was repeated 30 times. This reaction system was further diluted 50-fold with tricin-EDTA buffer and using 2.5 μl of the dilution as a template and the changed primer combination of AP1 and PDN, the reaction was conducted at 94 C. for one minute, followed by 4 cycles of 94 C.×1 minute, 98 C.×10 seconds and 72 C.×5 minutes, 4 cycles of 98 C.×10 seconds and 70 C.×5 minutes, and 26 cycles of 98 C.×10 seconds and 68 C.×5 minutes. The amplification product was electrophoresed on 1.2% agarose gel and stained with ethidium bromide and a band of about 150 bp was cut out and centrifugally filtered through a centrifugal filter tube (Millipore), extracted with phenol, and precipitated from ethanol. The recovered DNA was subcloned into plasmid vector PCRTMII according to the manual of TA Cloning Kit (Invitrogen). The vector was then introduced into E. coli JM109 and the resulting transformant was cultured and the sequence of the inserted cDNA fragment was analyzed as in Example 18. As a result, the sequence shown in FIG. 23 was obtained. Based on this sequence, primers FB (SEQ ID NO:34) and FG (SEQ ID NO:35) were synthesized and the 3′ sequence was cloned (3′ RACE). Using the same template as that for 5′ RACE in the same quantity and the combination of the accompanying adapter primer AP1 with the primer FC, PCR was carried out at 94 C. for 1 minute, followed by 5 cycles of 98 C.×10 seconds and 72 C.×5 minutes, 5 cycles of 98 C.×10 seconds and 70 C.×5 minutes, and 25 cycles of 98 C.×10 seconds and 68 C.×5 minutes. Then, using 2.5 μl of a 50-fold dilution of this reaction mixture in tricin-EDTA buffer as the template and the combination of the accompanying primer AP2 with the primer FB, the reaction was further conducted at 94 C. for one minute, followed by 4 cycles of 98 C.×10 seconds and 72 C.×5 minutes, 4 cycles of 98 C.×10 seconds and 70 C.×5 minutes, and 27 cycles of 98 C.×10 seconds and 68 C.×5 minutes. The amplification product was electrophoresed on 1.2% agarose gel and stained with ethidium bromide and a band of about 400 bp was cut out and the DNA was recovered as in 5′-RACE. This DNA fragment was subcloned into plasmid vector pCRTMII and introduced into E. coli JM109 and the sequence of the inserted cDNA fragment in the resulting transformant was analyzed. From the results of 5′ RACE and 3′ RACE, the DNA sequence, FIG. 24 coding for the complete coding region of the bioactive polypeptide defined by SEQ ID NO:1 was established. Thus, in FIG. 24 (a) and (b), the base134 is G, the base184 is T or C, and the base245 was T or C.

The cDNA shown in FIG. 24 was the cDNA encoding a polypeptide consisting of 98 amino acids. The fact that the amino acids in 1-22-positions comprise a cluster of hydrophobic amino acids taken together with the fact that the N-terminal region of the active peptide begins with Ser in 23-position as shown in Example 14 suggested that the amino acids 1-22 represent a secretion signal sequence. On the other hand, the Gly Arg Arg Arg sequence in 54-57 positions of the polypeptide was found to be a typical amino acid sequence motif which exists in the event of cleavage of a bioactive peptide. As it is the case with this cleavage motif, it is known that because of the presence of Gly, the C-terminus of the product peptide is frequently amidated.

The P-3 N-terminal sequence data of Example 14 and P-2 N-terminal sequence data in Example 16 coupled with this GlyArgArgArg sequence suggest that at least the same of the bioactive peptides cut out from the polypeptide encoded by this cDNA are defined by SEQ ID NO:26, SEQ ID NO:27, SEQ ID NO:39, SEQ ID NO:40, SEQ ID NO:41, SEQ ID NO:42, SEQ ID NO:43 or SEQ ID NO :44.

### Example 20

### Acquisition of a DNA fragment comprising the full coding region of bovine-derived bioactive polypeptide cDNA by PCR

Using the CDNA prepared with Marathon CDNA amplification kit in Example 17 as a template, a DNA fragment including the entire coding region of bioactive polypeptide cDNA was constructed. First, based on the sequence of cDNA elucidated in Example 19, two primers having base sequences defined by SEQ ID NO:42 and SEQ ID NO:43, respectively, were synthesized. BOVF 5′-GTGTCGACGAATGAAGGCGGTGGGGGCCTGGC-3′ (SEQ ID NO:36) BOVR (24 mer) 5′-AGGCTCCCGCTGTTATTCCTGGAC-3′ (SEQ ID NO:37)

BOVF contains the initiation codon of bioactive polypeptide cDNA and is a sense sequence corresponding to -2-+22 (A of the initiation codon ATG being reckoned as +1) with restriction enzyme SalI site added. On the other hand, BOVR is an antisense sequence corresponding to +285-+309 which includes the termination codon of bioactive polypeptide cDNA.

The PCR was conduced as follows. The cDNA prepared using Marathon cDNA amplification kit in Example 17 was diluted 100-fold in tricin-EDTA buffer and using 2.5 μl of the dilution, a reaction mixture was prepared as in Example 2 and subjected to 94 C.×1 minute, 3 cycles of 98 C.×10 seconds and 72 C.×5 minutes, 3 cycles of 98 C.×10 seconds and 70 C.×5 minutes, and 27 cycles of 98 C.×10 seconds and 68 C.×5 minutes. The amplification product was subjected to 2% agarose electrophoresis and ethidium bromide staining and a band of about 320 bp was cut out. The DNA was recovered and subcloned in plasmid vector PCRTMII as in Example 3. The vector was introduced into Escherichia coli JM109 to provide the transformant E. coli JM109/pBOV3. The sequence of the CDNA fragment inserted in the transformant was then analyzed. As a result, this DNA fragment was confirmed to be a fragment covering the entire coding region of the bioactive polypeptide cDNA.

### Example 21

### Synthesis of Ser-Arg-Ala-His-Gln-His-Ser-Met-Glu-Ile-Arg-Thr-Pro-Asp-Ile-Asn-Pro-Ala-Trp-Tyr-Ala-Gly-Arg-Gly-Ile-Arg-Pro-Val-Gly-Arg-Phe-NH2 (19P2-L31) 1) Synthesis of Ser(Bzl)-Arg(Tos)-Ala-His(Bom)-Gln-His(Bom)-Ser(Bzl)-Met-Glu(OcHex)-Ile-Arg(Tos)-Thr(Bzl)-Pro-Asp(OcHex)-Ile-Asn-Pro-Ala-Trp(CHO)-Tyr(Br-Z)-Ala -Gly-Arg-(Tos)-Gly-Ile-Arg(Tos)-Pro-Val-Gly-Arg(Tos)-Phe- pMBHA-resin

The reactor of a peptide synthesizer (Applied Biosystems 430A) was charged with 0.71 g (0.5 mmole) of commercial p-methyl-BHA resin (Applied Biosystems, currently Perkin Elmer). After wetting with DCM, the initial amino acid Boc-Phe was activated by the HOBt/DCC method and introduced into the p-methyl-BHA resin. The resin was treated with 50% TFA/DCM to remove Boc and make the amino group free and neutralized with DIEA. To this amino group was condensed the next amino acid Boc-Arg (Tos) by the HOBt/DCC method. After the absence of unreacted amino function was verified by ninhydrin test, a sequential condensation of Boc-Gly, Boc-Val, Boc-Pro, Boc-Arg(Tos), Boc-Ile, Boc-Gly, Boc-Arg(Tos), Boc-Gly, Boc-Ala, Boc-Tyr(Br-Z) was carried out. The Boc-Ala and Boc-Tyr (Br-Z), the condensation of which was found insufficient by ninhydrin test, was recondensed to complete the reaction. The resin was dried and a half of the resin was withdrawn. To the remainder, Boc-Trp(CHO), Boc-Ala, Boc-Pro, Boc-Asn, Boc-Ile, Boc-Asp(OcHex), Boc-Pro, Boc-Thr(Bzl), Boc-Arg(Tos), Boc-Ile, Boc-Glu(OcHex), Boc-Met, Boc-Ser(Bzl), Boc- His(Bom), Boc-Gln, Boc-His(Bom), Boc-Ala, Boc-Arg(Tos), Boc-Ser(Bzl) were serially condensed and recondensed until sufficient condensation was confirmed by ninhydrin test. After introduction of the full sequence of amino acids of 19P2-L31, the resin was treated with 50% TFA/DCM to remove Boc groups on the resin and, then, dried to provide 1.28 g of the peptide resin. 2) Synthesis of Ser-Arg-Ala-His-Gln-His-Ser-Met-Glu- Ile-Arg-Thr-Pro-Asp-Ile-Asn-Pro-Ala-Trp-Tyr-Ala-Gly- Arg-Gly-Ile-Arg-Pro-Val-Gly-Arg-Phe-NH₂(19P2-L31)

In a Teflon hydrogen fluoride reactor, the resin obtained in 1) was reacted with 3.8 g of p-cresol, 1 ml of 1,4-butanedithiol, and 10 ml of hydrogen fluoride at 0 C. for 60 minutes. The hydrogen fluoride and 1,4- butanedithiol (1 ml) were distilled off under reduced pressure and the residue was diluted with 100 ml of diethyl ether, stirred, filtered through a glass filter, and the fraction on the filter was dried. This fraction was suspended in 50 ml of 50% acetic acid/ H₂O and stirred to extract the peptide. After separation of the resin, the extract was concentrated under reduced pressure to about 5 ml and chromatographed on Sephadex G-25 (2×90 cm). Development was carried out with 50% acetic acid/ H₂O and the 114 ml - 181 ml fraction was pooled and lyophilized to recover 290 mg of white powders containing 19P2-L31. The powders were applied to a reversed-phase column of LiChroprep RP-18 (Merck) and repeatedly purified by gradient elution using 0.1% TFA/ H₂O and 0.1% TFA-containing 30% acetonitrile/H₂O. The fraction eluted at about 25% acetonitrile was pooled and lyophilized to provide 71 mg of white powders. Mass spectrum (M+H)+3574.645 HPLC elution time 18.2 min. Column conditions

Column: Wakosil 5C18 (4.6×100 mm)

Eluent: A (0.1% TFA/ H₂O)

B (0.1% TFA-containing 50 (% acetonitrile/ H₂O)

Linear gradient elution from A to B (25 min.) Flow rate: 1.0 ml/min.

### Example 22

### Synthesis of Ser-Arg-Ala-His-Gln-His-Ser-Met(O)-Glu-Ile-Arg-Thr-Pro-Asp-Ile-Asn-Pro-Ala-Trp-Tyr-Ala-Gly-Arg-Gly-Ile-Arg-Pro-Val-Gly-Arg-Phe-NH2(19P2-L31(O))

In 20 ml of 5% acetic acid/H₂O was dissolved 6 mg of synthetic 19P2-L31 and the Met only was selectively oxidized with 40 μl of 30% H₂O₂. After completion of the reaction, the reaction mixture was immediately applied to a reversed-phase column of LiChroprep RP-18 (Merck) for purification to provide 5.8 mg of the objective peptide. Mass spectrum (M+H)+3590.531 HPLC elution time 17.9 min. Column conditions

Column: Wakosil 5C18 (4.6×100 mm)

Eluent: A (0.1% TFA/ H₂O)

B (0.1% TFA-containing 50% acetonitrile/ H₂O)

Linear gradient elution from A to B (25 min.) Flow rate: 1.0 ml/min.

### Example 23

### Synthesis of Thr-Pro-Asp-Ile-Asn-Pro-Ala-Trp-Tyr-Ala-Gly-Arg-Gly-Ile-Arg-Pro-Val-Gly-Arg-Phe-NH2(19P2-L20)

To the resin subjected to condensations up to Boc- Tyr(Br-Z) in Example 21-1) was further condensed with Boc-Trp(CHO), Boc-Ala, Boc-Pro, Boc-Asn, Boc-Ile, Boc- Asp(OcHex), Boc-Pro and Boc-Thr(Bzl) serially in the same manner to provide 1.14 g of Boc-Thr(Bzl)-Pro-Asp(OcHex)-Ile-Asn-Pro-Ala-Trp(CHO)-Tyr(Br-Z)-Ala-Gly-Arg(Tos)-Gly-Ile-Arg(Tos)-Pro-Val-Gly-Arg(Tos)-Phe-PMBHA-resin. This resin was treated with hydrogen fluoride and purified column chromatography in the same manner as Example 21-2) to provide 60 mg of white powders. Mass spectrum (M+H)+2242.149 HPLC elution time 10.4 min. Column conditions

Column: Wakosil 5C18 (4.6×100 mm)

Eluent: A (0.1% TFA-containing 15% acetonitrile/H₂O)

B (0.1% TFA-containing 45% acetonitrile/H₂O)

Linear gradient elution from A to B (15 min.) Flow rate: 1.0 ml/min.

### Example 24

### Determination of arachidonic acid metabolites-releasing activity of synthetic peptide (19P2-L31)

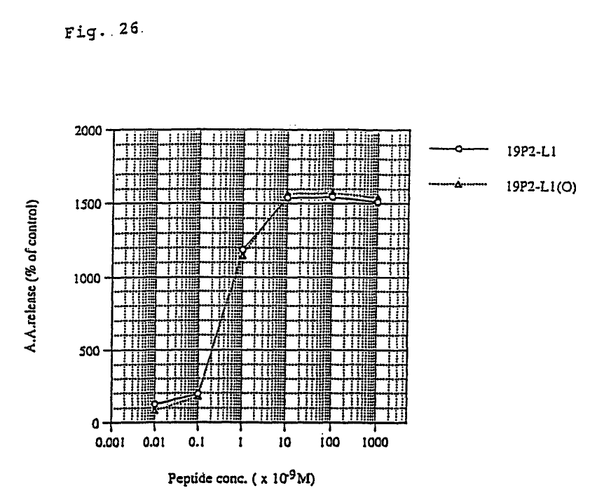

The activity of the peptide (19P2-L31) synthesized in Example 21 to specifically release arachidonic acid metabolites from CHO-19P2 cells was assayed in the same manner as Example 11. The synthetic peptide was dissolved in degassed distilled water at a concentration of 10⁻³M and diluted with 0.05% BSA-HBSS and the activity to promote release of arachidonic acid metabolites from CHO-19P2 cells at each concentration was assayed using the amount of [³H] arachidonic acid metabolites as the indicator. As a result, concentration-dependent arachidonic acid metabolite-releasing activity was detected over the range of 10⁻¹²M -10⁻⁶M, FIG. 25. When the arachidonic acid metabolite-releasing activity of peptide 19P2-L31(O), i.e. the methionine-oxidation product of 19P2-L31 synthesized in Example 22, was compared with that of 19P2-L31, it was found that the activity of 19P2-L31(O) was equivalent to the activity of 19P2-L31 as can be seen from FIG. 26.

### Example 25

### Determination of arachidonic acid metabolites-releasing activity of synthetic peptide (19P2-L20)

The activity of the synthetic equivalent (19P2- L20) of natural peptide P-2 as synthesized in Example 23 to specifically promote release of arachidonic acid metabolites from CHO-19P2 cells was determined as in Example 11. Thus, the synthetic peptide was dissolved in degassed distilled water at a final concentration of 10⁻³M and this solution was serially diluted with 0.05% BAS-HBSS. The activity to specifically promote release of arachidonic acid metabolites from CHO-19P2 cells at each concentration was assayed using the amount of [³H] arachidonic acid metabolites as the indicator.

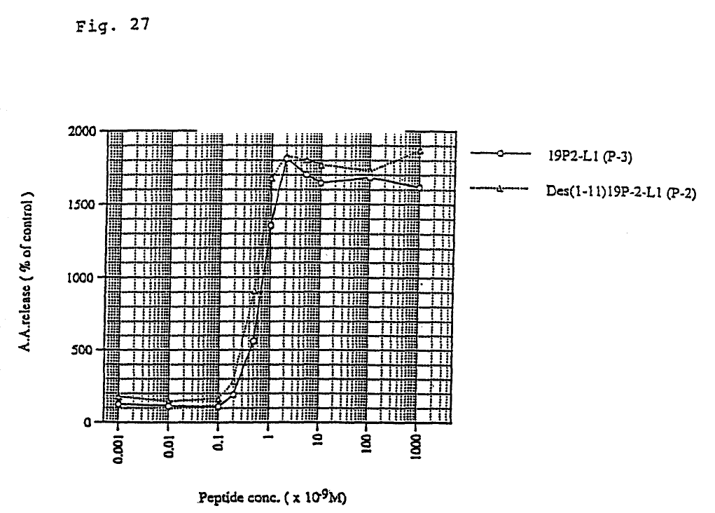

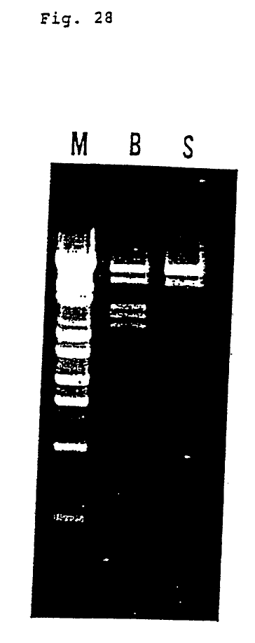

As a result, concentration-dependent arachidonic acid metabolite-releasing activity was detected over the range of 10⁻¹² -10⁻⁶M in nearly the same degree as 19P2-L31, FIG. 27.

### Example 26

### Analysis of the coding region base sequence of bovine genomic DNA

pBOV3 was digested with restriction enzyme EcoRI and after fractionation by agarose gel electrophoresis, the DNA corresponding to the cDNA fragment was recovered to prepare a probe. This DNA was labeled with ³²P using a multiprime DNA labeling kit (Amersham). About 2.0×10⁶ phages of Bovine Genomic Library (Clontech BL1015j) constructed using cloning vector EMBL3 SP6/T7 and Escherichia coli K802 as the host were seeded in an LB agar plate and cultured overnight for plaque formation. The it plaques were transferred to a nitrocellulose filter and after alkaline modification and neutralization, heat-treated (80 C., 2 hours) to inactivate the DNA. This filter was incubated with the labeled probe in 50% formamide-Hybri buffer (50% formamide, 5×Denhardt solution, 4×SSPE, 0.1 mg/ml heat-denatured salmon sperm DNA, 0.1% SDS) at 42 C. overnight for hybridization. After this hybridization, the filter was washed with 2×SSC, 0.1% SDS at room temperature for 1.5 hours, and further washed in the same buffer at 55 C. for 30 minutes. Detection of the clone hybridizing with the probe was carried out on Kodak X-ray film (X-OMATTMAR) after 4 days of exposure using a sensitization screen at −80 C. After development of the film, the film was collated with plate positions and the phages which had hybridized were recovered. Then, plating and hybridization were repeated in the same manner for cloning of the pharges.

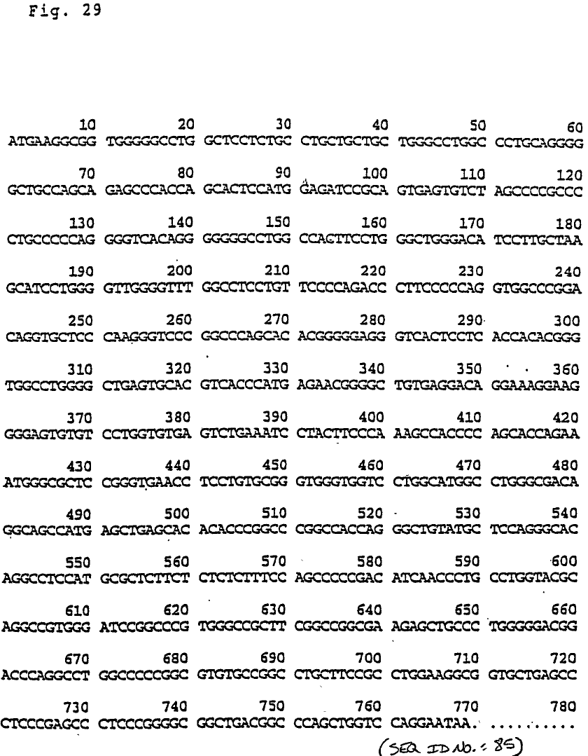

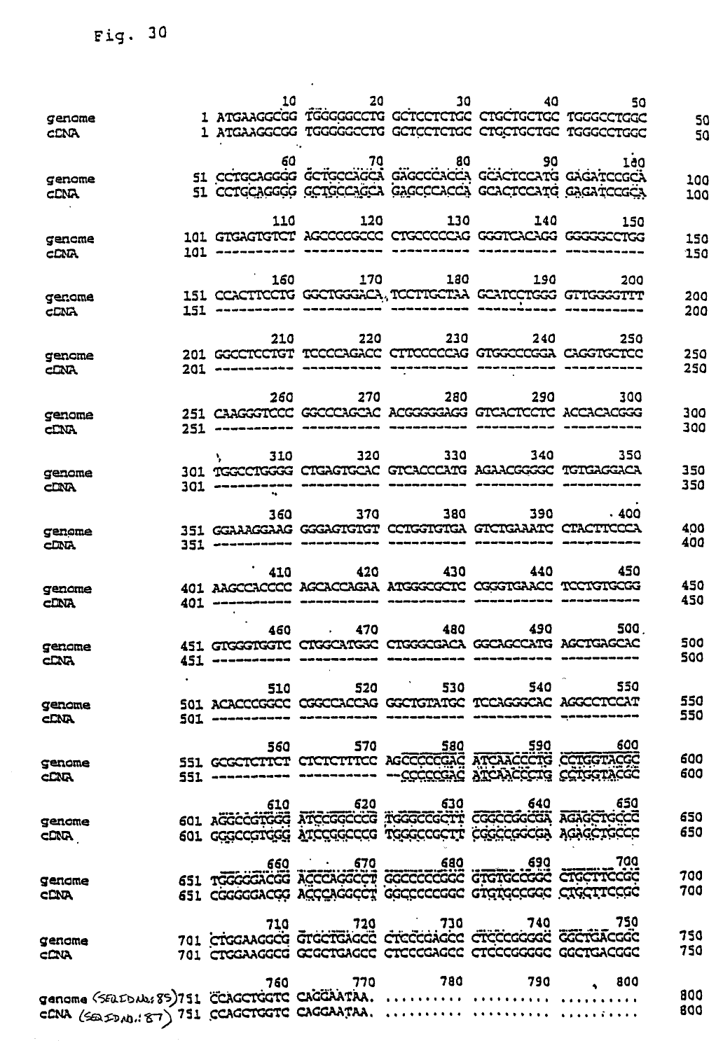

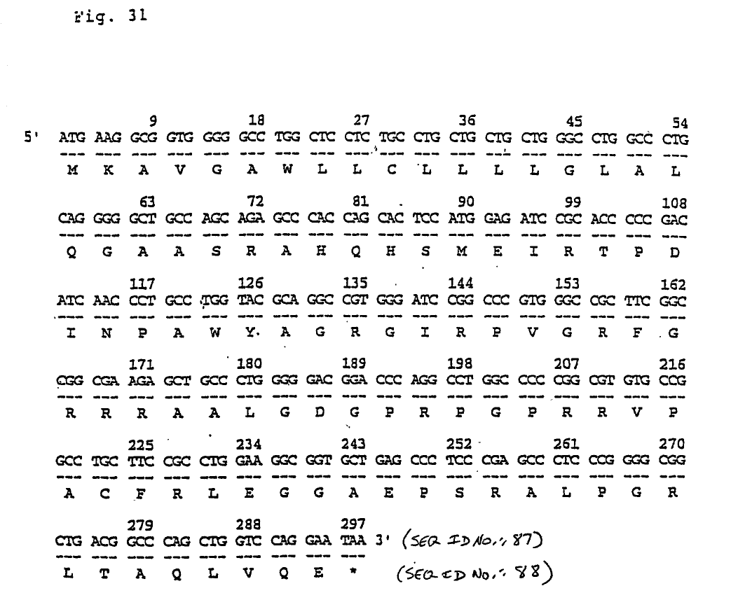

The cloned phages were prepared on a large scale by the plate lysate method and the phage DNA was extracted. Then, cleavage at the restriction enzyme SalI and BamHI cleavage sites at both ends of the cloning site of the vector and detection of the inserted fragment derived from bovine genomic DNA was carried out by 1.2% agarose gel electrophoresis FIG. 28. As a result, in the case of BamHI digestion, 3 fragments were detected in addition to the bands derived from the phages. In the case of SalI digestion, one band overlapping the phage band was detected. The SalI-digested fragment being considered to harbor the full length and in order to subclone this fragment into a plasmid vector, it was ligated to BAP (E. coli-derived alkaline phosphatase)-treated plasmid vector pUC18 (Pharmacia) and introduced into E. coli JM109. From this microorganism, a genome-derived SalI fragment-inserted plasmid DNA was prepared on a production scale and the base sequence in the neighborhood of its coding region was analyzed using Perkin Elmer Applied Biosystems 370 Å fluorecent sequencer and the same manufacturer's kit. As a result, the sequence shown in FIG. 29 was obtained. Comparison with the coding region of cDNA reveals that because of its being derived from genomic DNA, the coding region is divided in two by a 472 bp intron, FIG. 30. FIG. 31 and SEQ ID NO:44 present the amino acid sequence predicted from this bovine genome coding region (excluding the intron region).

### Example 27

### Preparation of rat medulla oblongata poly(A+)RNA fraction and synthesis of cDNA

Using Isogen (Nippon Gene), total RNA was prepared from the dorsal region of rat medulla oblongata and using FastTrack (Invitrogen), poly(A)+RNA fraction was prepared. To 5 μg of this poly(A)+RNA was added the primer random DNA hexamer (BRL) and using Moloney mouse leukemia reverse transcriptase (BRL) and the accompanying buffer, complementary DNA was synthesized. The reaction product was precipitated from ethanol and dissolved in 12 μl of distilled water. In addition, from 1 μg of this poly(A)+RNA, a cDNA was synthesized using Marathon cDNA amplification kit (Clontech) according to the manual and dissolved in 10 μl of DW.

### Example 28

### Acquisition of rat bioactive polypeptide cDNA by RACE

To obtain the full coding region of rat bioactive polypeptide cDNA, an experiment was performed in the same manner as the acquisition of bovine cDNA. First, PCR was carried out using the same primers P5-1 (SEQ ID NO:29) and P3-1 (SEQ ID NO:30) as used in Example 18 as primers and the complementary DNA synthesized in Example 27 using the primer random DNA hexamer (BRL) and Moloney mouse leukemia reverse transcriptase (BRL) as a template. The reaction system was composed of 1.25 μl of the template cDNA, 200 μM of dNTP, 1 μM each of the primers, ExTaq (Takara Shuzo Co., Japan) as DNA polymerase, and 2.5 μl of the accompanying buffer, with a sufficient amount of water to make a total of 25 μl. The reaction was carried out at 94 C. for 1 minute, followed by 40 cycles of 98C.×10 seconds, 50 C.×30 seconds, and 72 C.×5 seconds, and the reaction mixture was then allowed to stand at 72 C. for 20 seconds. The thermal cycler used was GeneAmp2400 (Perkin Elmer). The amplification product was subjected to 4% agarose gel electrophoresis and ethidium bromide staining and the band of about 80 bp was cut out. Then, in the manner described in Example 19, the DNA was recovered, subcloned into plasmid vector pCRTMII, and introduced into E. coli JM109, and the inserted cDNA fragment was sequenced. As a result, a partial sequence of rat bioactive polypeptide could be obtained. Based on this sequence, two primers, namely RA (SEQ ID NO:45) for 3′ RACE and RC (SEQ ID NO:46) for 5′ RACE were synthesized and 5′ and 3′ RACEs were carried out.

RA:5′-CARCAYTCCATGGAGACAAGAACCCC-3′

(where R means A or G; Y means T or G) (SEQ ID NO:45)

RC:5′-TACCAGGCAGGATTGATACAGGGG-3′ (SEQ ID NO:46)

As a template, the template synthesized using Marathon cDNA amplification kit (Clontech) in Example 27 was diluted 40-fold with the accompanying tricin-EDTA buffer and 2.5 μl of the dilution was used. As primers, RA and the adapter primer AP1 accompanying the kit were used for 3′ RACE, and RC and AP1 for 5′ RACE. The reaction mixture was prepared in otherwise the same manner as above. The reaction conditions were 94 C.×1 minute, 5 cycles of 98 C.×10 seconds and 72 C.×45 seconds, 3 cycles of 98 C.×10 seconds and 70 C.×45 seconds, and 40 cycles of 98 C.×10 seconds and 68 C.×45 seconds. As a result, a band of about 400 bp was obtained from 3′ RACE and bands of about 400 bp and 250 bp from 5′ RACE. These bands were recovered in the same manner as above and using them as templates and the primers used in the reaction, sequencing was carried out with Dye Terminator Cycle Sequencing Kit (ABI). As a result, the sequence up to poly A could be obtained from the region considered to be the 5′ noncoding region.

### Example 29

### Acquisition of the full-length cDNA of rat bioactive polypeptide by PCR

Based on the sequence obtained in Example 28, two primers, viz. rF for the region including the initiation codon (SEQ ID NO:47) and rR for the 3′ side from the termination codon (SEQ ID NO:48), were synthesized to amplify the fragment including the full- length CDNA.

rF:5′-GGCATCATCCAGGAAGACGGAGCAT-3′ (SEQ ID NO:47)

rR:5′-AGCAGAGGAGAGGGAGGGTAGAGGA-3′ (SEQ ID NO:48)

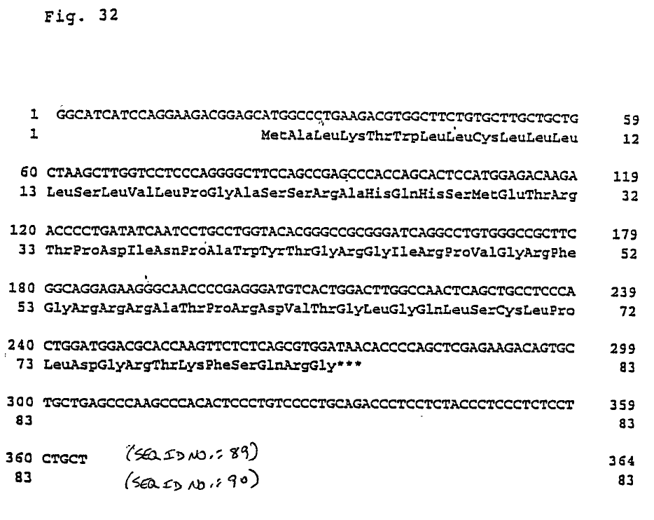

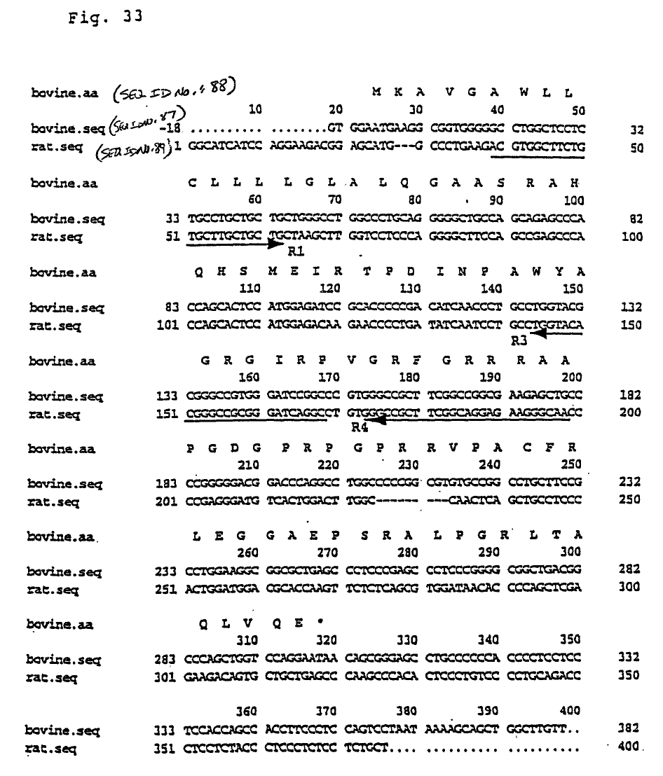

Using the cDNA prepared using Moloney mouse leukemia reverse transcriptase in Example 27 as a template and ExTaq (Takara Shuzo Co., Japan), PCR was carried out by repeating 40 cycles of 95 C.×30 seconds and 68 C.×60 seconds. The amplification product was subjected to agarose gel electrophoresis and ethidium bromide staining and a band of about 350 bp was cut out. The DNA was recovered, subcloned into plasmid vector PCRTMII, and introduced into E. coli JM109 as in Example 19. The plasmid was extracted from the transformant and the base sequence was determined. As a result, E. coli JM 109/pRAV3 having the full-length cDNA of rat bioactive polypeptide was obtained, FIG. 32.

### Example30

### Synthesis of cDNA from the human total brain poly(A)+RNA fraction

From 1 μg of human total brain poly(A)+RNA fraction (Clontech), cDNA was synthesized with Marathon cDNA amplification kit (Clontech) according to the manual and dissolved in 10 μl. In addition, the random DNA hexamer (BRL) was added as primer to 5 μg of the same poly(A)+RNA fraction and using Moloney mouse leukemia reverse transcriptase (BRL) and the accompanying buffer, complementary DNA was synthesized. The reaction product was precipitated from ethanol and dissolved in 30 μl of Tris EDTA.

### Example31

### Acquisition of human bioactive polypeptide CDNA by RACE

From the amino acid sequence of rat bioactive polypeptide established in Example 28, FIG. 33, the well-preserved regions of rat and bovine polypeptides were selected and the following 3 primers R1 (SEQ ID NO:49), R3 (SEQ ID NO:50), and R4 (SEQ ID NO:51) were synthesized. Then, amplification of the region flanked by them was attempted by PCR using human cDNA as a template. Referring to FIG. 33, “bovine. aa” represents the amino acid sequence of bovine polypeptide, “bovine. seq” represents the base sequence of the DNA coding for bovine polypeptide, and “rat. seq” represents the base sequence of the DNA coding for rat polypeptide.

R1:5′-ACGTGGCTTCTGTGCTTGCTGC-3′ (SEQ ID NO:49)

R3:5′-GCCTGATCCCGCGGCCCGTGTACCA-3′ (SEQ ID NO:50)

R4:5′-TTGCCCTTCTCCTGCCGAAGCGGCCC-3′ (SEQ ID NO:51)

The cDNA prepared using Marathon cDNA amplification kit (Clontech) in Example 30 was diluted 30-fold with tricin-EDTA buffer and 0.25 μl of the dilution was used as a template. The reaction mixture was composed of 200 μM of dNTP, 0.2 μM each of the primers R1 and R4, a 50:50 mixture of Taq Start Antibody (Clontech) and DNA polymerase ExTaq (Takara Shuzo Co., Japan), 2.5 μl of the accompanying buffer, and a sufficient amount of water to make a total volume of 25 μl. The reaction conditions were 94° C.×1 minute, followed by 42 cycles of 98° C.×10 seconds and 68° C.×40 seconds, and 1 minute of standing at 72° C. Then, using 1 μl of a 100-fold dilution of the above reaction mixture in tricine-EDTA buffer as a template, the same reaction mixture as above except that the primer combination was changed to R1 and R3 was prepared and PCR was carried out in the sequence of 94° C.×1 minute and 25 cycles of 98° C.×10 seconds and 68° C.×40 seconds. The amplification product was subjected to 4% agarose gel electrophoresis and ethidium bromide staining. As a result, a band of about 130 bp was obtained as expected. This band was recovered in the same manner as in Example 28 and using the recovered fragment as a template, sequencing was carried out with Dye Terminator Cycle Sequencing Kit (ABI). As a result, a partial sequence of human bioactive polypeptide could be obtained. Therefore, based on this sequence, primers HA (SEQ ID NO:52) and HB (SEQ ID NO:53) were synthesized for 3′ RACE and primers HE (SEQ ID NO:54) and HF (SEQ ID NO:55) for 5′ RACE and 5′ and 3′ RACEs were carried out.

HA:5′-GGCGGGGGCTGCAAGTCGTACCCATCG-3′ (SEQ ID NO:52)

HB:5′-CGGCACTCCATGGAGATCCGCACCCCT-3′ (SEQ ID NO:53)

HE:5′-CAGGCAGGATTGATGTCAGGGGTGCGG-3′ (SEQ ID NO:54)

HF:5′-CATGGAGTGCCGATGGGTACGACTTGC-3′ (SEQ ID NO:55)

As the template, 2.5 μl of a 20-fold dilution of the cDNA prepared in Example 30 in tricin-EDTA buffer was used. For the initial PCR, reaction mixtures were prepared in the same manner as above except that HA and adapter primer AP1 were used for 3′ RACE and HE and AP1 for 5′ RACE. The reaction sequence was 94° C.×1 minute, 5 cycles of 98° C.×10 seconds and 72° C. for 35 seconds, 5 cycles of 98° C.×10 seconds and 70° C.×35 seconds, and 40 cycles of 98° C.×10 seconds and 68° C.×35 seconds. Then, using 1 μl of a 100-fold dilution of this reaction mixture in tricin-EDTA buffer as a template, a second PCR was carried out in the same cycles as the first PCR. However, the reaction mixture was prepared using primers HB and AP1 for 3′ RACE or HF and AP2 for 5′ RACE and Klen Taq (Clontech) as DNA polymerase and the accompanying buffer. As a result, a band of about 250 bp was obtained from 3′ RACE and a band of about 150 bp from 5′-RACE. These bands were sequenced by the same procedure as above and using them in combination with the partial sequence obtained previously, the sequence from the region presumed to be 5′-noncoding region to poly(A) of human bioactive polypeptide was obtained.

### Example 32

### Acquisition of human bioactive polypeptide full-length cDNA by PCR

Based on the sequence obtained in Example 31, two primers 5H (SEQ ID NO:56) and 3HN (SEQ ID NO:57) were synthesized for amplification of a fragment including full-length cDNA.

5H:5′-GGCCTCCTCGGAGGAGCCAAGGGATGA-3′ (SEQ ID NO:56)

3HN:5′-GGGAAAGGAGCCCGAAGGAGAGGAGAG-3′ (SEQ ID NO:57)

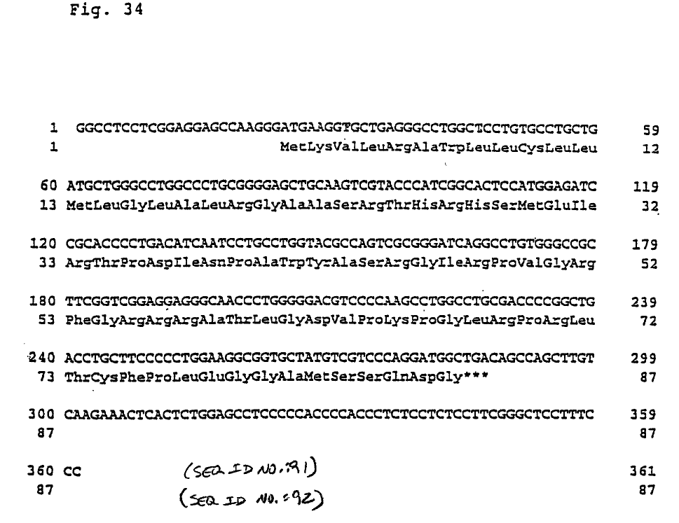

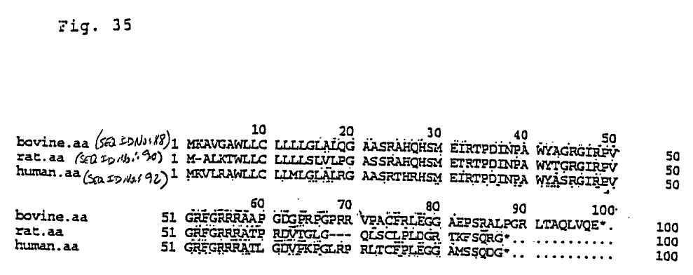

Using 2.5 μl of the cDNA prepared using Moloney mouse leukemia reverse transcriptase (BRL) in Example 30 as a template and the reaction mixture prepared using Klen Taq DNA polymerase (Clontech), the PCR reaction was conducted in the sequence of 94° C.×1 minute and 40 cycles of 98° C.×10 seconds and 68° C.×30 seconds. The fragment of about 360 bp obtained was recovered and subcloned (pCRTM 2.1 was used as the vector) in the same manner as Example 29. The plasmid was recovered and its base sequence was determined. As a result, E. coli JM109/pHOV7 harboring the human bioactive polypeptide full-length cDNA was obtained, FIG. 34. In regard to the amino acid sequence of the translation region, a comparison was made between this human bioactive polypeptide and the bovine polypeptide shown in Example 20 or the rat polypeptide in Example 29, FIG. 35.

### Example 33

### Acquisition and sequencing of a DNA including the ligand polypeptide coding region from the murine genomic DNA

Based on the sequence (FIG. 32) of the CDNA coding for the rat ligand polypeptide obtained in Example 29, the following two primers were synthesized.

rFBG:5′-AGATTGGCATCATCCAGGAAGACGGAGCAT-3′ (SEQ ID NO:61)

rRSA:5′-GTCGACTCAGCAGCACTGTCTTCTCGAGCTG-3′ (SEQ ID NO:62)

Using the above two primers and, as a template, 0.5 ng of murine genomic DNA (Mouse BALB/c genomic DNA), a PCR amplification was carried out.

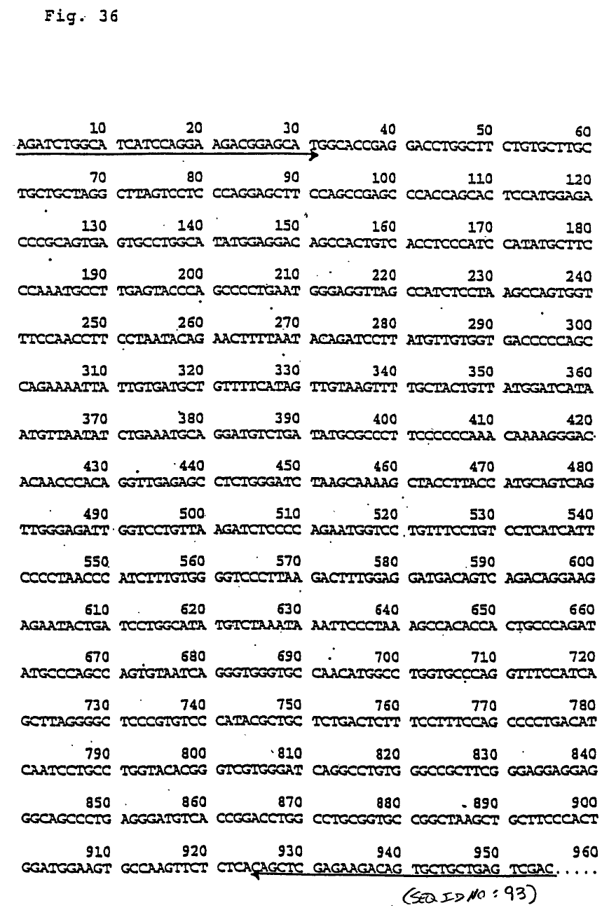

The reaction components used were: 200 nM each synthetic DNA primers, 0.5 ng template DNA, 0.5 μl of 0.25 mM dNTPs ExTaq polymerase, and enzyme-attached buffer, total volume 50 μl. Using a thermal cycler (Perkin-Elmer), an amplification reaction was carried out in 30 cycles of 30 seconds at 95° C. and 60 seconds at 67° C. Identification of the amplification product was made by 1.2% agarose gel electrophoresis and ethidium bromide staining, and a band of about 1 kb was recovered and subcloned using TA Cloning Kit (Invitrogen). This ligation mixture was used to transform Escherichia coli JM109 and clones harboring the inserted fragment were selected in LB agar containing ampicillin and X-gal. A white-colored clone was picked out to obtain a transformant Escherichia coli JM109/pmGB3. This clone was cultured overnight in ampicillin-containing LB medium and the plasmid DNA was prepared using an automatic plasmid extraction apparatus. Using a portion of the DNA thus prepared, a sequencing reaction was carried out using ABI Dye Terminator Cycle Sequencing Kit (ABI), and after decoding with a fluorescent automated sequencer, the nucleotide sequence data was analyzed with DNASIS (Hitachi System Engineering) FIG. 36. The underlined sequences correspond to the primers.

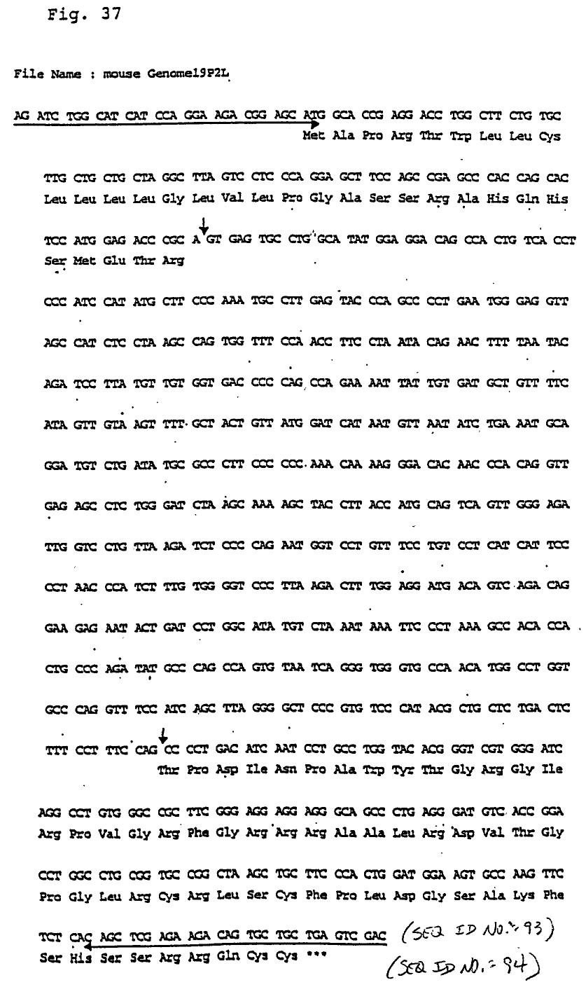

The nucleotide sequence thus determined was compared with the sequence of SEQ ID NO:58, 59, or 60. As a result, it was found that the DNA fragment in the plasmid pmGB3 harbored by Escherichia coli JM109/pmGB3 codes for a novel murine ligand polypeptide, FIG. 37.

### Example 34

### Acquisition and Sequencing of the full-length translated region DNA of the ligand polypeptide coding region of mouse genomic DNA

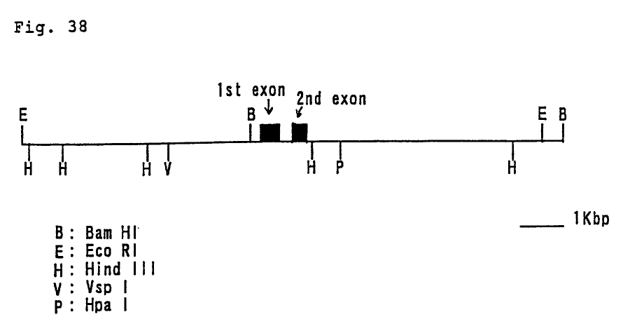

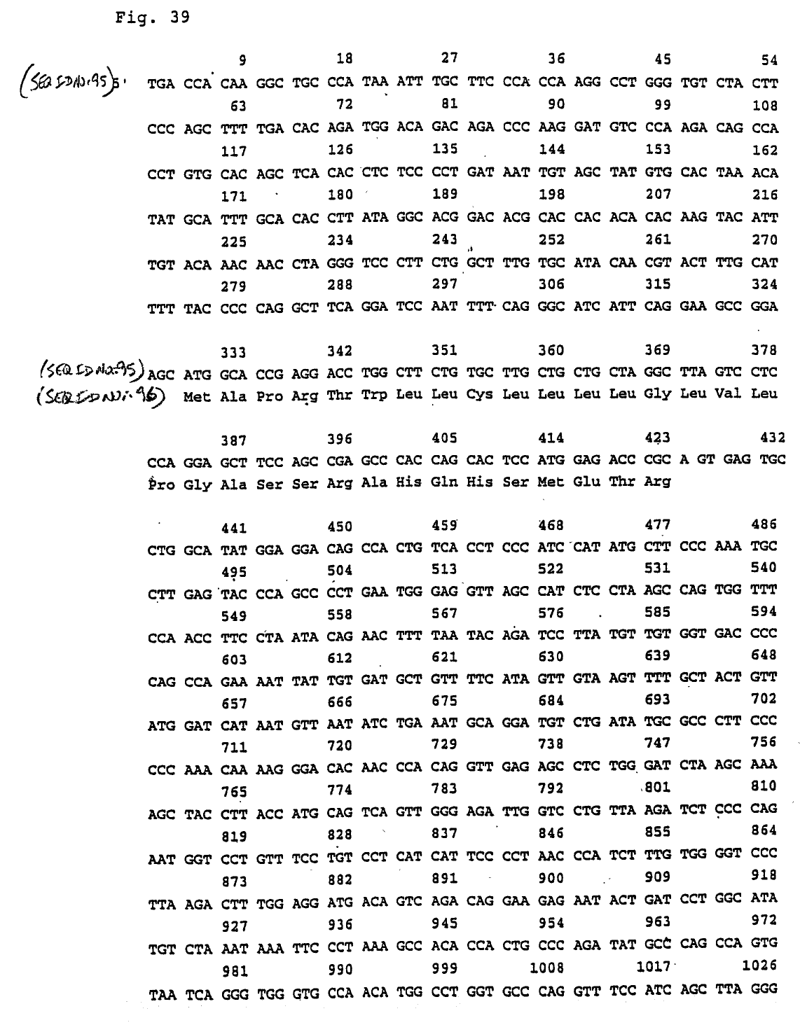

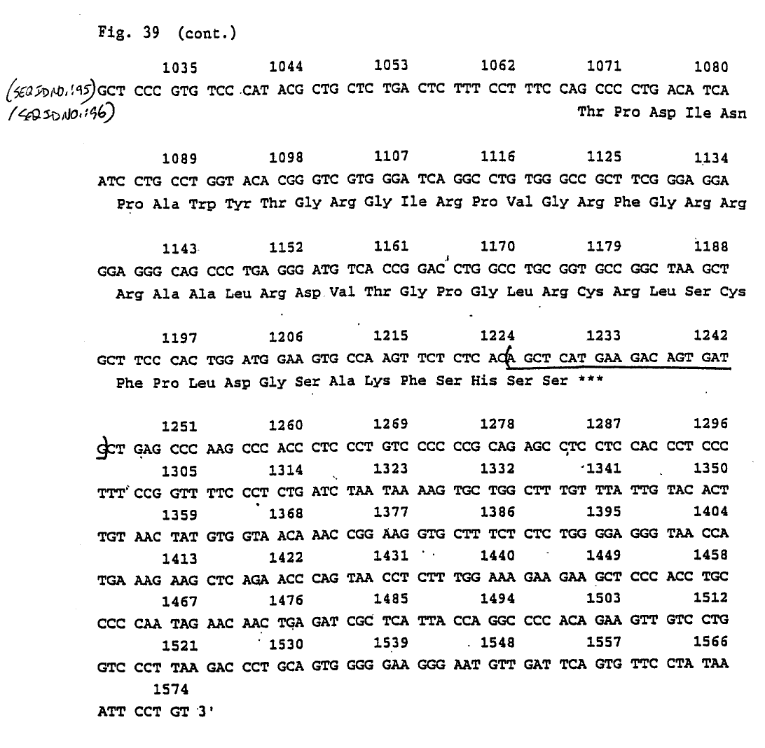

The DNA fragment contained in the plasmid pmGB3 as obtained in Example 33 was prepared and sent to Genome Systems with a request for hybridization screening from Mouse ES129/SuJ BAC library using the fragment as the probe (Catalogue BAC, 4921). From the BAC clone received from Genome Systems, the DNA fragment containing the objective ligand peptide coding region was subcloned in the EcoRI site of pUC18 Vector and JM109/pmGFEI was obtained. Then, the sequence of the region of interest was similarly determined. The determined sequence (FIG. 38) coded for the indicated amino sequence. The sequence in parentheses differs from that obtained in Example 33. While the latter was the PCR primer used, the sequence obtained this time was shorter by 5 residues.

### Example 35

### Construction of a targeting vector for production of knockout mice

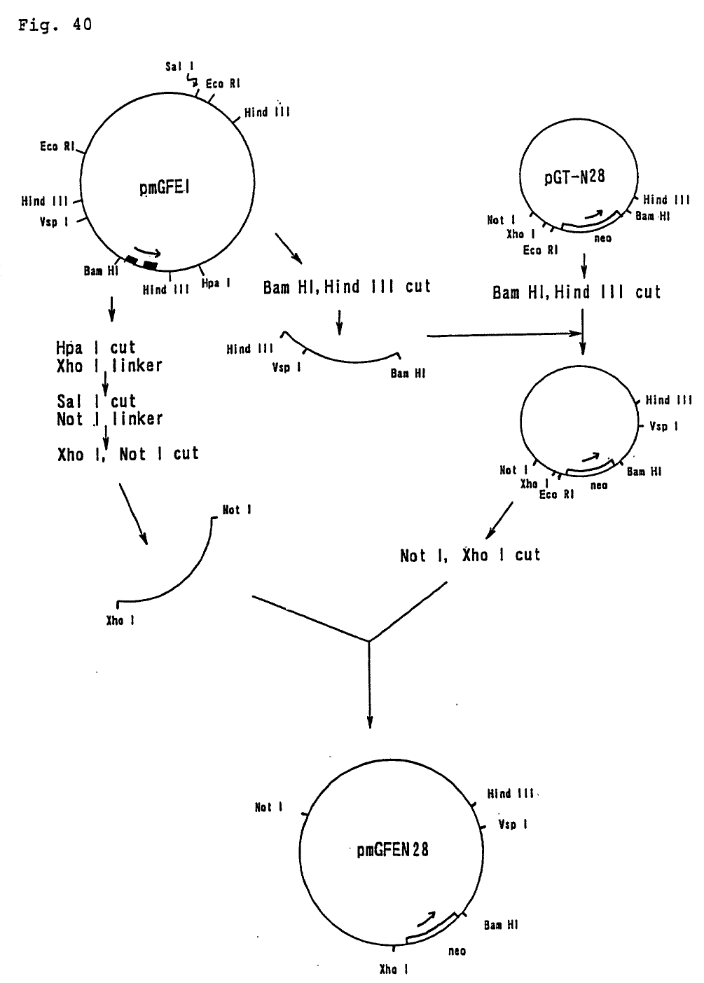

The targeting vector was constructed using pmGFE1 obtained in Example 34 and pGT-N28 (NEB). The BamHI- Hind III fragment including Vsp1 site was inserted between the BamHI site and Hind III site of pGT-N28 (5′ upstream region of the peptide gene). Then, the HpaI- SalI fragment (3′-downstream region of the peptide gene) was inserted, after modification to XhoI-NotI by linker ligation, between the XhoI and NotI sites to provide pmGFEN28 (FIG. 40).

### Example 36

### Acquisition of homologous recombinant ES cells

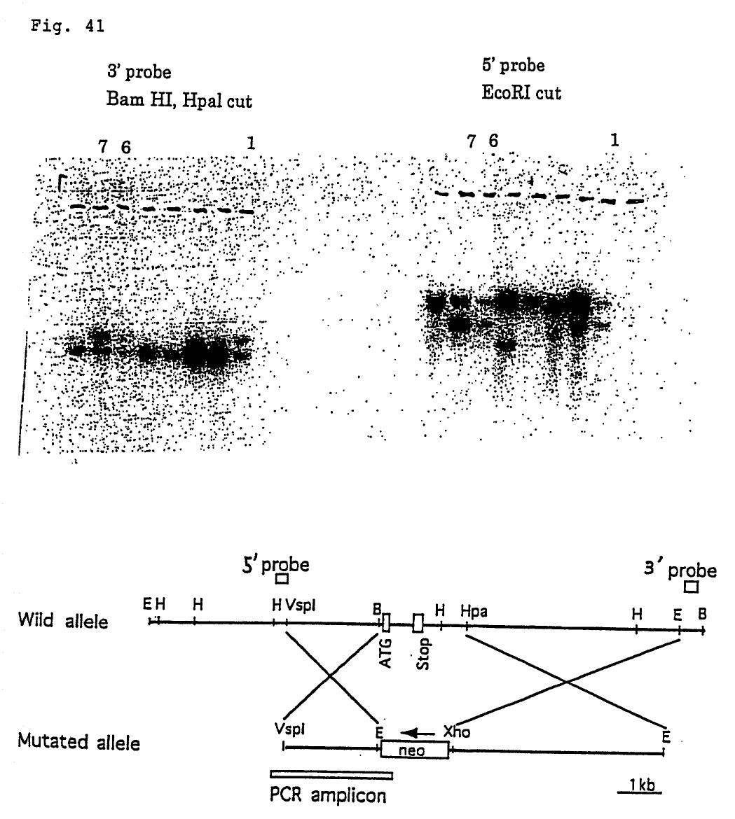

Acquisition of homologous recombinant ES cells was carried out using ES cells (RW-4) purchased from Genome Systems in accordance with the attahed manual. Specifically, a 13 kbp (approx.) DNA fragment available upon VspI/NotI digestion of the pmGFEN28 obtained in Example 35 was isolated by agarose gel electrophoresis and, using Bio-Rad Gene Pulser, electroporated into RW- 4 cells to obtain recombinant cells. In addition, the same DNA fragment was sent to Genome Systems with a request for electroporation and performed an antibiotic selection of ES cells (MK 2/20). Thus, as shown in FIG. 41, Southern blotting gave the objective homologous recombinant clones No. 92 Hinuma, No. 56FF, and No. 81FF from lane 1, lane 6, and lane 7 where 12 kbp and 5.5 kbp bands could be detected with the 5′- probe and 7.5 kbp and 5.5 kbp bands with the 3′-probe. Using those ES clones, chimera mice can be constructed.

### Example 37

### Bioactivity of ligand peptides of the invention

Specific interaction of synthetic peptides of the invention with hGR3 and UHR-1 receptor causes a specific arachidonic acid metabolite release from CHO-19P2 cells.

Prolactin (PRL) release by cultured RC-4B/C cells is promoted by administration of peptide of the invention.

1. A polypeptide comprising an amino acid sequence represented by SEQ ID NO:1, or a substantial equivalent thereto, or its amide or ester, or a salt thereof.

2. A DNA comprising a DNA having a nucleotide sequence coding for the polypeptide according to claim 1.

3. A DNA according to claim 2, which comprises a nucleotide sequence represented by SEQ ID NO:2 or SEQ ID NO:3.

4. A recombinant vector comprising the DNA according to claim 2.

5. A transformant which is transformed by the DNA according to claim 2 or the recombinant vector according to claim 4.

6. A non-human knock out animal having an inactivated DNA of the DNA according to claim 2.

7. A non-human transgenic animal having the DNA according to claim 2 or its mutein, or the recombinant vector according to claim 4.

8. A non-human animal cell having an inactivated DNA of the DNA according to claim 2.

9. A method for producing the non-human animal cell according to claim 8, which comprises introducing an inactivated DNA of the DNA according to claim 2.

10. A method for producing the polypeptide according to claim 1 or its amide or ester, or the salt thereof, which comprises cultivating the transformant according to claim 5 to produce and accumulate the polypeptide according to claim 1, and collecting the same.

11. A pharmaceutical composition which comprises the polypeptide according to claim 1 or its amide or ester, or the salt thereof.

12. An antibody against the polypeptide according to claim 1 or its amide or ester, or the salt thereof.


SEQUENCE LISTING (96 sequences)

SEQ ID NO: 1 - protein, 82 aa, Murine
APRTWLLCLLLLGLVLPGASSRAHQHSMETRTPDINPAWYTGRGIRPVGRFGRRRAALRDVTGPGLRCRLSCFPLDGSAKFS

SEQ ID NO: 2 - DNA, 249 bp, Murine
ggcaccgaggacctggcttctgtgcttgctgctgctaggcttagtcctcccaggagcttccagccgagcccaccagcactccatggagacccgcacccctgacatcaatcctgcctggtacacgggtcgtgggatcaggcctgtgggccgcttcgggaggaggagggcagccctgagggatgtcaccggacctggcctgcggtgccggctaagctgcttcccactggatggaagtgccaagttctctca

SEQ ID NO: 3 - DNA, 893 bp, Murine
ggcaccgaggacctggcttctgtgcttgctgctgctaggcttagtcctcccaggagcttccagccgagcccaccagcactccatggagacccgcagtgagtgcctggcatatggaggacagccactgtcacctcccatccatatgcttcccaaatgccttgagtacccagcccctgaatgggaggttagccatctcctaagccagtggtttccaaccttcctaatacagaacttttaatacagatccttatgttgtggtgacccccagccagaaaattattgtgatgctgttttcatagttgtaagttttgctactgttatggatcataatgttaatatctgaaatgcaggatgtctgatatgcgcccttccccccaaacaaaagggacacaacccacaggttgagagcctctgggatctaagcaaaagctaccttaccatgcagtcagttgggagattggtcctgttaagatctccccagaatggtcctgtttcctgtcctcatcattcccctaacccatctttgtggggtcccttaagactttggaggatgacagtcagacaggaagagaatactgatcctggcatatgtctaaataaattccctaaagccacaccactgcccagatatgcccagccagtgtaatcagggtgggtgccaacatggcctggtgcccaggtttccatcagcttaggggctcccgtgtcccatacgctgctctgactctttcctttccagcccctgacatcaatcctgcctggtacacgggtcgtgggatcaggcctgtgggccgcttcgggaggaggagggcagccctgagggatgtcaccggacctggcctgcggtgccggctaagctgcttcccactggatggaagtgccaagttctctca

SEQ ID NO: 4 - protein, 31 aa, Murine
SRAHQHSMETRTPDINPAWYTGRGIRPVGRF

SEQ ID NO: 5 - protein, 31 aa, Artificial Sequence; identical to SEQ ID NO: 4
  PEPTIDE (1)..(31): antigen

SEQ ID NO: 6 - protein, 20 aa, Artificial Sequence
  PEPTIDE (1)..(20): antigen
TPDINPAWYTGRGIRPVGRF

SEQ ID NO: 7 - protein, 9 aa, Artificial Sequence
  PEPTIDE (1)..(9): antigen
SRAHQHSME

SEQ ID NO: 8 - protein, 9 aa, Artificial Sequence
  PEPTIDE (1)..(9): antigen
TPDINPAWY

SEQ ID NO: 9 - protein, 10 aa, Artificial Sequence
  PEPTIDE (1)..(10): antigen
GRGIRPVGRF

SEQ ID NO: 10 - protein, 91 aa, Human
LVLVIARVRRLHNVTNFLIGNLALSDVLMCTACVPLTLAYAFEPRGWVFGGGLCHLVFFLQPVTVYVSVFTLTTIAVDRYVVLVHPLRRRI

SEQ ID NO: 11 - protein, 59 aa, Human
GLLLVTYLLPLLVILLSYVRVSVKLRNRVVPGCVTQSQADWDRARRRRTFCLLVVVVVV

SEQ ID NO: 12 - protein, 370 aa, Human
MASSTTRGPRVSDLFSGLPPAVTTPANQSAEASAGNGSVAGADAPAVTPFQSLQLVHQLKGLIVLLYSVVVVVGLVGNCLLVLVIARVRRLHNVTNFLIGNLALSDVLMCTACVPLTLAYAFEPRGWVFGGGLCHLVFFLQPVTVYVSVFTLTTIAVDRYVVLVHPLRRRISLRLSAYAVLAIWALSAVLALPAAVHTYHVELKPHDVRLCEEFWGSQERQRQLYAWGLLLVTYLLPLLVILLSYVRVSVKLRNRVVPGCVTQSQADWDRARRRRTFCLLVVVVVVFAVCWLPLHVFNLLRDLDPHAIDPYAFGLVQLLCHWLAMSSACYNPFIYAWLHDSFREELRKLLVAWPRKIAPHGQNMTVSVVI

SEQ ID NO: 13 - protein, 206 aa, Murine
LVLVIARVRRLYNVTNFLIGNLALSDVLMCTACVPLTLAYAFEPRGWVFGGGLCHLVFFLQAVTVYVSVFTLTTIAVDRYVVLVHPLRRRISLRLSAYAVLAIWVLSAVLALPAAVHTYHVELKPHDVRLCEEFWGSQERQRQLYAWGLLLVTYLLPLLVILLSYARVSVKLRNRVVPGRVTQSQADWDRARRRRTFCLLVVVVVV

SEQ ID NO: 14 - protein, 126 aa, Murine
VVLVHPLRRRISLRLSAYAVLGIWALSAVLALPAAVHTYHVELKPHDVSLCEEFWGSQERQRQIYAWGLLLGTYLLPLLAILLSYVRVSVKLRNRVVPGSVTQSQADWDRARRRRTFCLLVVVVVV

SEQ ID NO: 15 - DNA, 273 bp, Human
ctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccggtacgtcgtgctggtgcacccgctgaggcggcgcatc

SEQ ID NO: 16 - DNA, 177 bp, HUMAN
ggcctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtg

SEQ ID NO: 17 - DNA, 1110 bp, HUMAN
atggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatc

SEQ ID NO: 18 - DNA, 618 bp, Murine
ctggtgctggtgatcgcgcgggtgcgccggctgtacaacgtgacgaatttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcaggcggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggtgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgcccgggtgtcagtgaagctccgcaaccgcgtggtgccgggccgcgtgacccagagccaggccgactgggaccgcgctcggcgccggcgcaccttctgcttgctggtggtggtcgtggtggtg

SEQ ID NO: 19 - DNA, 378 bp, Murine
gtggttctggtgcacccgctacgtcggcgcatttcactgaggctcagcgcctacgcggtgctgggcatctgggctctatctgcagtgctggcgctgccggccgcggtgcacacctaccatgtggagctcaagccccacgacgtgagcctctgcgaggagttctggggctcgcaggagcgccaacgccagatctacgcctgggggctgcttctgggcacctatttgctccccctgctggccatcctcctgtcttacgtacgggtgtcagtgaagctgaggaaccgcgtggtgcctggcagcgtgacccagagtcaagctgactgggaccgagcgcgtcgccgccgcactttctgtctgctggtggtggtggtggtagtg

SEQ ID NO: 20 - DNA, 25 bp, Artificial Sequence
  misc_feature (1)..(25): mixed synthetic primer described on pgs. 94-5
cgtggscmtsstgggcaacnycctg

SEQ ID NO: 21 - DNA, 27 bp, Artificial Sequence
  misc_feature (1)..(27): mixed synthetic primer described on pg. 95
gtngwrrggcanccagcagakggcaaa

SEQ ID NO: 22 - DNA, 27 bp, Artificial Sequence
  misc_feature (1)..(27): mixed synthetic primer described on pg. 98
ctgtgygysatygcnntkgaymgstac

SEQ ID NO: 23 - DNA, 29 bp, Artificial Sequence
  misc_feature (1)..(29): mixed synthetic primer described on pg. 98
akgwagwagggcagccagcagansrygaa

SEQ ID NO: 24 - DNA, 24 bp, Artificial Sequence
  misc_feature (1)..(24): synthetic primer described on pg. 106
ctgacttattttctgggctgccgc

SEQ ID NO: 25 - DNA, 24 bp, Artificial Sequence
  misc_feature (1)..(24): synthetic primer described on pg. 106
aacaccgacacatagacggtgacc

SEQ ID NO: 26 - protein, 29 aa, Bovine
SRAHQHSMEIRTPDINPAWYAGRGIRPVG

SEQ ID NO: 27 - protein, 19 aa, Bovine
TPDINPAWYAGRGIRPVGR

SEQ ID NO: 28 - protein, 98 aa, Murine
MKAVGAWLLCLLLLGLALQGAASRAHQHSMEIRTPDINPAWYAGRGIRPVGRFGRRRAAPGDGPRPGPRRVPACFRLEGGAEPSRALPGRLTAQLVQE

SEQ ID NO: 29 - DNA, 20 bp, Artificial Sequence
  misc_feature (1)..(20): synthetic primer described on pg. 113
gcncaycarcaytgyatgga

SEQ ID NO: 30 - DNA, 26 bp, Artificial Sequence
  misc_feature (1)..(26): synthetic primer described on pg. 113
ccnacgggnckdatgccnckgccngc

SEQ ID NO: 31 - DNA, 26 bp, Artificial Sequence
  misc_feature (1)..(26): synthetic primer described on pg. 113
acgggcckdatgccnckgccngcrta

SEQ ID NO: 32 - DNA, 20 bp, Artificial Sequence
  misc_feature (1)..(20): synthetic primer
ccggcgtaccaggcagggtt

SEQ ID NO: 33 - DNA, 28 bp, Artificial Sequence
  misc_feature (1)..(28): synthetic primer
aggcagggttgatgtcgggggtgcggat

SEQ ID NO: 34 - DNA, 27 bp, Artificial Sequence
  misc_feature (1)..(27): synthetic primer
ctgccagcagagcccaccagcactcca

SEQ ID NO: 35 - DNA, 27 bp, Artificial Sequence
  misc_feature (1)..(27): synthetic primer
gtgggggcctggctcctctgcctgctg

SEQ ID NO: 36 - DNA, 32 bp, Artificial Sequence
  misc_feature (1)..(32): synthetic primer
gtgtcgacgaatgaaggcggtgggggcctggc

SEQ ID NO: 37 - DNA, 24 bp, Artificial Sequence
  misc_feature (1)..(24): synthetic primer
aggctcccgctgttattcctggac

SEQ ID NO: 38 - protein, 98 aa, Bovine
MKAVGAWLLCLLLLGLALQGAASRAHQHSMEIRTPDINPAWYAGRGIRPVGRFGRRRAALGDGPRPGPRRVPACFRLEGGAEPSRALPGRLTAQLVQE

SEQ ID NO: 39 - protein, 31 aa, Bovine
SRAHQHSMEIRTPDINPAWYAGRGIRPVGRF

SEQ ID NO: 40 - protein, 32 aa, Bovine
SRAHQHSMEIRTPDINPAWYAGRGIRPVGRFG

SEQ ID NO: 41 - protein, 33 aa, Bovine
SRAHQHSMEIRTPDINPAWYAGRGIRPVGRFGR

SEQ ID NO: 42 - protein, 20 aa, Bovine
TPDINPAWYAGRGIRPVGRF

SEQ ID NO: 43 - protein, 21 aa, Bovine
TPDINPAWYAGRGIRPVGRFG

SEQ ID NO: 44 - protein, 22 aa, Bovine
TPDINPAWYAGRGIRPVGRFGR

SEQ ID NO: 45 - DNA, 26 bp, Artificial Sequence
  misc_feature (1)..(26): synthetic primer
carcaytccatggagacaagaacccc

SEQ ID NO: 46 - DNA, 24 bp, Artificial Sequence
  misc_feature (1)..(24): synthetic primer
taccaggcaggattgatacagggg

SEQ ID NO: 47 - DNA, 25 bp, Artificial Sequence
  misc_feature (1)..(25): synthetic primer
ggcatcatccaggaagacggagcat

SEQ ID NO: 48 - DNA, 25 bp, Artificial Sequence
  misc_feature (1)..(25): synthetic primer
agcagaggagagggagggtagagga

SEQ ID NO: 49 - DNA, 22 bp, Artificial Sequence
  misc_feature (1)..(22): synthetic primer
acgtggcttctgtgcttgctgc

SEQ ID NO: 50 - DNA, 25 bp, Artificial Sequence
  misc_feature (1)..(25): synthetic primer
gcctgatcccgcggcccgtgtacca

SEQ ID NO: 51 - DNA, 26 bp, Artificial Sequence
  misc_feature (1)..(26): synthetic primer
ttgcccttctcctgccgaagcggccc

SEQ ID NO: 52 - DNA, 27 bp, Artificial Sequence
  misc_feature (1)..(27): synthetic primer
ggcgggggctgcaagtcgtacccatcg

SEQ ID NO: 53 - DNA, 27 bp, Artificial Sequence
  misc_feature (1)..(27): synthetic primer
cggcactccatggagatccgcacccct

SEQ ID NO: 54 - DNA, 27 bp, Artificial Sequence
  misc_feature (1)..(27): synthetic primer
caggcaggattgatgtcaggggtgcgg

SEQ ID NO: 55 - DNA, 27 bp, Artificial Sequence
  misc_feature (1)..(27): synthetic primer
catggagtgccgatgggtacgacttgc

SEQ ID NO: 56 - DNA, 27 bp, Artificial Sequence
  misc_feature (1)..(27): synthetic primer
ggcctcctcggaggagccaagggatga

SEQ ID NO: 57 - DNA, 27 bp, Artificial Sequence
  misc_feature (1)..(27): synthetic primer
gggaaaggagcccgaaggagaggagag

SEQ ID NO: 58 - DNA, 294 bp, Bovine
atgaaggcggtgggggcctggctcctctgcctgctgctgctgggcctggccctgcagggggctgccagcagagcccaccagcactccatggagatccgcacccccgacatcaaccctgcctggtacgcrggccgtgggatccggcccgtgggccgcttcggccggcgaagagctgcccygggggacggacccaggcctggcccccggcgtgtgccggcctgcttccgcctggaaggcggygctgagccctcccgagccctcccggggcggctgacggcccagctggtccaggaa

SEQ ID NO: 59 - DNA, 249 bp, Murine
atggccctgaagacgtggcttctgtgcttgctgctgctaagcttggtcctcccaggggcttccagccgagcccaccagcactccatggagacaagaacccctgatatcaatcctgcctggtacacgggccgcgggatcaggcctgtgggccgcttcggcaggagaagggcaaccccgagggatgtcactggacttggccaactcagctgcctcccactggatggacgcaccaagttctctcagcgtgga

SEQ ID NO: 60 - DNA, 261 bp, Human
atgaaggtgctgagggcctggctcctgtgcctgctgatgctgggcctggccctgcggggagctgcaagtcgtacccatcggcactccatggagatccgcacccctgacatcaatcctgcctggtacgccagtcgcgggatcaggcctgtgggccgcttcggtcggaggagggcaaccctgggggacgtccccaagcctggcctgcgaccccggctgacctgcttccccctggaaggcggtgctatgtcgtcccaggatggc

SEQ ID NO: 61 - DNA, 30 bp, Artificial Sequence
  misc_feature (1)..(30): synthetic primer
agattggcatcatccaggaagacggagcat

SEQ ID NO: 62 - DNA, 31 bp, Artificial Sequence
  misc_feature (1)..(31): synthetic primer
gtcgactcagcagcactgtcttctcgagctg

SEQ ID NO: 63 - DNA, 297 bp, Homo sapiens
  CDS (1)..(297): cDNA fragment from clone p19P2 depicted in FIG 1.
gtgggcatggtgggcaacgtcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccggtacgtcgtgctggtgcacccgctgaggcggcgcatc

SEQ ID NO: 64 - protein, 99 aa, Homo sapiens
  misc_feature (1)..(24): Primer 21M13
VGMVGNVLLVLVIARVRRLHNVTNFLIGNLALSDVLMCTACVPLTLAYAFEPRGWVFGGGLCHLVFFLQPVTVYVSVFTLTTIAVDRYVVLVHPLRRRI

SEQ ID NO: 65 - DNA, 204 bp, Homo sapiens
  CDS (1)..(204): cDNA fragment in clone p19P2 depicted in FIG 2.
ggcctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgtttgccatctgctggttgccttactac

SEQ ID NO: 66 - protein, 68 aa, Homo sapiens
  misc_feature (178)..(204): Primer M13Rv-N
GLLLVTYLLPLLVILLSYVRVSVKLRNRVVPGCVTQSQADWDRARRRRTFCLLVVVVVVFAICWLPYY

SEQ ID NO: 67 - protein, 99 aa, mammalian; identical to SEQ ID NO: 64
  PEPTIDE (1)..(99): p19P2 translated sequence depicted in FIG 5 from aa 1-99

SEQ ID NO: 68 - protein, 68 aa, mammalian; identical to SEQ ID NO: 66
  peptide (1)..(68): Translated peptide fragment from p19P2 depicted in FIG 5 aa 156-2 30

SEQ ID NO: 69 - protein, 227 aa, mammalian
  peptide (1)..(227): Amino acid peptide sequence of S12863 depicted in FIG 5.
LGVSGNLALIIIILKQKEMRNVTNILIVNLSFSDLLVAVMCLPFTFVYTLMDHWVFGETMCKNPFVQCVSITVSIFSLVLIAVERHELIINPRGWRPNNRHAYIGITVIWVLAVASSLPFVIYQILTDEPFQNVSLAAFKDKYVCFDKFPSDSHRLSYTTLLLVLQYFGPLCFIFICYFKIYIRLKRRNNMMDKIRDSKYRSSETKRINVMLLSIVVAFAVCWLPLT

SEQ ID NO: 70 - DNA, 669 bp, mammalian
  CDS (1)..(669): MIN6 derived cDNA fragment from clones pG3-2 and pG1-10 depicted in FIG 6
gtgggcatggtgggcaacatcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccggtacgtcgtgctggtgcacccgctgaggcggcgcatctcgctgcgcctcagcgcctacgctgtgctggccatctgggtgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgcccgggtgtcagtgaagctccgcaaccgcgtggtgccgggccgcgtgacccagagccaggccgactgggaccgcgctcggcgccggcgcaccttctgcttgctggtggtggtcgtggtggtgttcaccctctgctggctgcccttcttc

SEQ ID NO: 71 - protein, 223 aa, mammalian
  misc_feature (1)..(24): primer
VGMVGNILLVLVIARVRRLHNVTNFLIGNLALSDVLMCTACVPLTLAYAFEPRGWVFGGGLCHLVFFLQPVTVYVSVFTLTTIAVDRYVVLVHPLRRRISLRLSAYAVLAIWVLSAVLALPAAVHTYHVELKPHDVRLCEEFWGSQERQRQLYAWGLLLVTYLLPLLVILLSYARVSVKLRNRVVPGRVTQSQADWDRARRRRTFCLLVVVVVVFTLCWLPFF

SEQ ID NO: 72 - protein, 223 aa, mammalian
  PEPTIDE (1)..(223): Pg3-2/Pg1-10 fragments as translated MIN6 derived peptide compared with p19P2  as depicted in FIG 7
VGMVGNILLVLVIARVRRLYNVTNFLIGNLALSDVLMCTACVPLTLAYAFEPRGWVFGGGLCHLVFFLQAVTVYVSVFTLTTIAVDRYVVLVHPLRRRISLRLSAYAVLAIWVLSAVLALPAAVHTYHVELKPHDVRLCEEFWGSQERQRQLYAWGLLLVTYLLPLLVILLSYARVSVKLRNRVVPGRVTQSQADWDRARRRRTFCLLVVVVVVFTLCWLPFF

SEQ ID NO: 73 - DNA, 1331 bp, Homo sapiens
  CDS (118)..(1227): cDNA insert of phGR3 depicted in FIG 9.
catcgtcaagcagatgaagatcatccacgaggatggctactccgagggccagcagaaattctgccccttcttcccgcgagtgctttcccgctctccaaaccccactcccaggtggccatggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactcttggtcgcttggccccgcaagatagccccccatggccagaatatgaccgtcagcgtggtcatctgatgccacttagccaggccttggtcaaggagctccacttcaactggcctcctagggcaccactcgaggtcaatctggtgcttattctcagcaccagagctagc

SEQ ID NO: 74 - protein, 370 aa, Homo sapiens; identical to SEQ ID NO: 12

SEQ ID NO: 75 - DNA, 434 bp, mammalian
  CDS (1)..(432): MIN6-derived cDNA fragment of clone p5S38 as depicted in FIG 12.
ctgtgtgtcatcgcggtggataggtacgtggttctggtgcacccgctacgtcggcgcatttcactgaggctcagcgcctacgcggtgctgggcatctgggctctatctgcagtgctggcgctgccggccgcggtgcacacctaccatgtggagctcaagccccacgacgtgagcctctgcgaggagttctggggctcgcaggagcgccaacgccagatctacgcctgggggctgcttctgggcacctatttgctccccctgctggccatcctcctgtcttacgtacgggtgtcagtgaagctgaggaaccgcgtggtgcctggcagcgtgacccagagtcaagctgactgggaccgagcgcgtcgccgccgcactttctgtctgctggtggtggtggtggtagtgttcacgctctgctggctgcccttctacct

SEQ ID NO: 76 - protein, 144 aa, mammalian
  misc_feature (1)..(27): primer
LCVIAVDRYVVLVHPLRRRISLRLSAYAVLGIWALSAVLALPAAVHTYHVELKPHDVSLCEEFWGSQERQRQIYAWGLLLGTYLLPLLAILLSYVRVSVKLRNRVVPGSVTQSQADWDRARRRRTFCLLVVVVVVFTLCWLPFY

SEQ ID NO: 77 - DNA, 76 bp, Bovine
  CDS (1)..(75): cDNA fragment depicted in FIG 22.
gcccaccagcactccatggagatccgcacccccgacatcaaccctgcctggtacgcgggccgtgggatccggcccg

SEQ ID NO: 78 - protein, 25 aa, Bovine
  misc_feature (1)..(20): primer
AHQHSMEIRTPDINPAWYAGRGIRP

SEQ ID NO: 79 - DNA, 126 bp, Bovine
  CDS (6)..(125): cDNA fragment as depicted in FIG 23.
gtggaatgaaggcggtgggggcctggctcctctgcctgctgctgctgggcctggccctgcagggggctgccagcagagcccaccagcactccatggagatccgcacccccgacatcaaccctgcct

SEQ ID NO: 80 - protein, 40 aa, Bovine
  misc_feature (99)..(126): primer
MKAVGAWLLCLLLLGLALQGAASRAHQHSMEIRTPDINPA

SEQ ID NO: 81 - DNA, 380 bp, Bovine
  CDS (6)..(299): Bovine ligand polypeptide as depicted in FIG 24A.
gtggaatgaaggcggtgggggcctggctcctctgcctgctgctgctgggcctggccctgcagggggctgccagcagagcccaccagcactccatggagatccgcacccccgacatcaaccctgcctggtacgcrggccgtgggatccggcccgtgggccgcttcggccggcgaagagctgccccgggggacggacccaggcctggcccccggcgtgtgccggcctgcttccgcctggaaggcggygctgagccctcccgagccctcccggggcggctgacggcccagctggtccaggaataacagcgggagcctgccccccacccctcctcctccaccagccaccttccctccagtcctaataaaagcagctggcttgtt

SEQ ID NO: 82 - protein, 98 aa, Bovine; identical to SEQ ID NO: 28

SEQ ID NO: 83 - DNA, 380 bp, Bovine
  CDS (6)..(299): Bovine ligand polypeptide as depicted in FIG 24B.
gtggaatgaaggcggtgggggcctggctcctctgcctgctgctgctgggcctggccctgcagggggctgccagcagagcccaccagcactccatggagatccgcacccccgacatcaaccctgcctggtacgcrggccgtgggatccggcccgtgggccgcttcggccggcgaagagctgccctgggggacggacccaggcctggcccccggcgtgtgccggcctgcttccgcctggaaggcggygctgagccctcccgagccctcccggggcggctgacggcccagctggtccaggaataacagcgggagcctgccccccacccctcctcctccaccagccaccttccctccagtcctaataaaagcagctggcttgtt

SEQ ID NO: 84 - protein, 98 aa, Bovine; identical to SEQ ID NO: 38

SEQ ID NO: 85 - DNA, 769 bp, Bovine
  CDS (1)..(100): Bovine genomic DNA sequence as depicted in FIG 29.
atgaaggcggtgggggcctggctcctctgcctgctgctgctgggcctggccctgcagggggctgccagcagagcccaccagcactccatggagatccgcagtgagtgtctagccccgcccctgcccccaggggtcacagggggggcctggccacttcctgggctgggacatccttgctaagcatcctggggttggggtttggcctcctgttccccagacccttcccccaggtggcccggacaggtgctcccaagggtcccggcccagcacacgggggagggtcactcctcaccacacgggtggcctggggctgagtgcacgtcacccatgagaacggggctgtgaggacaggaaaggaaggggagtgtgtcctggtgtgagtctgaaatcctacttcccaaagccaccccagcaccagaaatgggcgctccgggtgaacctcctgtgcgggtgggtggtcctggcatggcctgggcgacaggcagccatgagctgagcacacacccggcccggccaccagggctgtatgctccagggcacaggcctccatgcgctcttctctctctttccagcccccgacatcaaccctgcctggtacgcrggccgtgggatccggcccgtgggccgcttcggccggcgaagagctgccctgggggacggacccaggcctggcccccggcgtgtgccggcctgcttccgcctggaaggcggygctgagccctcccgagccctcccggggcggctgacggcccagctggtccaggaataa

SEQ ID NO: 86 - protein, 98 aa, Bovine; identical to SEQ ID NO: 38

SEQ ID NO: 87 - DNA, 297 bp, Bovine
  CDS (1)..(294): Bovine genomic sequence with intron removed as depicted in FIG 31
atgaaggcggtgggggcctggctcctctgcctgctgctgctgggcctggccctgcagggggctgccagcagagcccaccagcactccatggagatccgcacccccgacatcaaccctgcctggtacgcrggccgtgggatccggcccgtgggccgcttcggccggcgaagagctgccctgggggacggacccaggcctggcccccggcgtgtgccggcctgcttccgcctggaaggcggygctgagccctcccgagccctcccggggcggctgacggcccagctggtccaggaataa

SEQ ID NO: 88 - protein, 98 aa, Bovine; identical to SEQ ID NO: 38

SEQ ID NO: 89 - DNA, 364 bp, Rat
  CDS (24)..(233): Rat derived ligand polypeptide sequence as depicted in FIG 32.
ggcatcatccaggaagacggagcatggccctgaagacgtggcttctgtgcttgctgctgctaagcttggtcctcccaggggcttccagccgagcccaccagcactccatggagacaagaacccctgatatcaatcctgcctggtacacgggccgcgggatcaggcctgtgggccgcttcggcaggagaagggcaaccccgagggatgtcactggacttggccaactcagctgcctcccactggatggacgcaccaagttctctcagcgtggataacaccccagctcgagaagacagtgctgctgagcccaagcccacactccctgtcccctgcagaccctcctctaccctccctctcctctgct

SEQ ID NO: 90 - protein, 70 aa, Rat
MALKTWLLCLLLLSLVLPGASSRAHQHSMETRTPDINPAWYTGRGIRPVGRFGRRRATPRDVTGLGQLSC

SEQ ID NO: 91 - DNA, 361 bp, Homo sapiens
  CDS (24)..(284): human ligand polypeptide cDNA sequence as depicted in FIG 34.
ggcctcctcggaggagccaagggatgaaggtgctgagggcctggctcctgtgcctgctgatgctgggcctggccctgcggggagctgcaagtcgtacccatcggcactccatggagatccgcacccctgacatcaatcctgcctggtacgccagtcgcgggatcaggcctgtgggccgcttcggtcggaggagggcaaccctgggggacgtccccaagcctggcctgcgaccccggctgacctgcttccccctggaaggcggtgctatgtcgtcccaggatggctgacagccagcttgtcaagaaactcactctggagcctcccccaccccaccctctcctctccttcgggctcctttccc

SEQ ID NO: 92 - protein, 87 aa, Homo sapiens
MKVLRAWLLCLLMLGLALRGAASRTHRHSMEIRTPDINPAWYASRGIRPVGRFGRRRATLGDVPKPGLRPRLTCFPLEGGAMSSQDG

SEQ ID NO: 93 - DNA, 955 bp, Mammalian
  misc_feature (1)..(31): primer
agatctggcatcatccaggaagacggagcatggcaccgaggacctggcttctgtgcttgctgctgctaggcttagtcctcccaggagcttccagccgagcccaccagcactccatggagacccgcagtgagtgcctggcatatggaggacagccactgtcacctcccatccatatgcttcccaaatgccttgagtacccagcccctgaatgggaggttagccatctcctaagccagtggtttccaaccttcctaatacagaacttttaatacagatccttatgttgtggtgacccccagccagaaaattattgtgatgctgttttcatagttgtaagttttgctactgttatggatcataatgttaatatctgaaatgcaggatgtctgatatgcgcccttccccccaaacaaaagggacacaacccacaggttgagagcctctgggatctaagcaaaagctaccttaccatgcagtcagttgggagattggtcctgttaagatctccccagaatggtcctgtttcctgtcctcatcattcccctaacccatctttgtggggtcccttaagactttggaggatgacagtcagacaggaagagaatactgatcctggcatatgtctaaataaattccctaaagccacaccactgcccagatatgcccagccagtgtaatcagggtgggtgccaacatggcctggtgcccaggtttccatcagcttaggggctcccgtgtcccatacgctgctctgactctttcctttccagcccctgacatcaatcctgcctggtacacgggtcgtgggatcaggcctgtgggccgcttcgggaggaggagggcagccctgagggatgtcaccggacctggcctgcggtgccggctaagctgcttcccactggatggaagtgccaagttctctcacagctcgagaagacagtgctgctgagtcgac

SEQ ID NO: 94 - protein, 91 aa, Mammalian
  misc_feature (1)..(31): primer
MAPRTWLLCLLLLGLVLPGASSRAHQHSMETRTPDINPAWYTGRGIRPVGRFGRRRAALRDVTGPGLRCRLSCFPLDGSAKFSHSSRRQCC

SEQ ID NO: 95 - DNA, 1574 bp, mammalian
  CDS (328)..(424): ligand polypeptide encoding nucleic acid depicted in FIG 39.
tgaccacaaggctgcccataaatttgcttcccaccaaggcctgggtgtctacttcccagcttttgacacagatggacagacagacccaaggatgtcccaagacagccacctgtgcacagctcacacctctcccctgataattgtagctatgtgcactaaacatatgcatttgcacaccttataggcacggacacgcaccacacacacaagtacatttgtacaaacaacctagggtcccttctggctttgtgcatacaacgtactttgcatttttacccccaggcttcaggatccaattttcagggcatcattcaggaagccggaagcatggcaccgaggacctggcttctgtgcttgctgctgctaggcttagtcctcccaggagcttccagccgagcccaccagcactccatggagacccgcagtgagtgcctggcatatggaggacagccactgtcacctcccatccatatgcttcccaaatgccttgagtacccagcccctgaatgggaggttagccatctcctaagccagtggtttccaaccttcctaatacagaacttttaatacagatccttatgttgtggtgacccccagccagaaaattattgtgatgctgttttcatagttgtaagttttgctactgttatggatcataatgttaatatctgaaatgcaggatgtctgatatgcgcccttccccccaaacaaaagggacacaacccacaggttgagagcctctgggatctaagcaaaagctaccttaccatgcagtcagttgggagattggtcctgttaagatctccccagaatggtcctgtttcctgtcctcatcattcccctaacccatctttgtggggtcccttaagactttggaggatgacagtcagacaggaagagattactgatcctggcatatgtctaaataaattccctaaagccacaccactgcccagatatgcccagccagtgtaatcagggtgggtgccaacatggcctggtgcccaggtttccatcagcttaggggctcccgtgtcccatacgctgctctgactctttcctttccagcccctgacatcaatcctgcctggtacacgggtcgtgggatcaggcctgtgggccgcttcgggaggaggagggcagccctgagggatgtcaccggacctggcctgcggtgccggctaagctgcttcccactggatggaagtgccaagttctctcacagctcatgaagacagtgatgctgagcccaagcccaccctccctgtccccccgcagagcctcctccaccctccctttccggttttccctctgatctaataaaagtgctggctttgtttattgtacacttgtaactatgtggtaacaaaccggaaggtgctttctctctggggagggtaaccatgaaagaagctcagaacccagtaacctctttggaaagaagaagctcccacctgcccccaatagaacaactgagatcgctcattaccaggccccacagaagttgtcctggtcccttaagaccctgcagtgggggaagggattgttgattcagtgttcctataaattcctgt

SEQ ID NO: 96 - protein, 86 aa, mammalian
  misc_feature (1224)..(1243): Bracket region depicted in FIG 39.
MAPRTWLLCLLLLGLVLPGASSRAHQHSMETRTPDINPAWYTGRGIRPVGRFGRRRAALRDVTGPGLRCRLSCFPLDGSAKFSHSS